(19) United States
(12) Patent Application Publication
(10) Pub. No.: US 2025/0100987 A1
(43) Pub. Date: Mar. 27, 2025

(54) MULTICYCLIC COMPOUNDS

(71) Applicant: ATLEE BIOTECH, INC., San Diego, CA (US)

(72) Inventor: Atlee Solomon, San Diego, CA (US)

(73) Assignee: ATLEE BIOTECH, INC., San Diego, CA (US)

(21) Appl. No.: 18/707,895

(22) PCT Filed: Nov. 11, 2022
(86) PCT No.: PCT/US2022/049719

§ 371 Date: May 7, 2024

Related U.S. Application Data
(60) Provisional application No. 63/279,094, filed on Nov. 13, 2021.
(60) Provisional application No. 63/282,264, filed on Nov. 23, 2021.
(60) Provisional application No. 63/299,749, filed on Jan. 14, 2022.
(60) Provisional application No. 63/317,980, filed on Mar. 9, 2022.
(60) Provisional application No. 63/321,915, filed on Mar. 21, 2022.
(60) Provisional application No. 63/336,476, filed on Apr. 29, 2022.
(60) Provisional application No. 63/375,050, filed on Sep. 8, 2022.

(51) Int. Cl. C07D 307/54; A61K 31/341; A61K 31/343; A61K 31/366; C07D 405/04; C07D 493/08

(52) U.S. Cl. CPC C07D 307/54; A61K 31/341; A61K 31/343; A61K 31/366; C07D 405/04; C07D 493/08

(57) ABSTRACT

Compounds of Formulae (I), (II) and (III), including pharmaceutically acceptable salts are described herein. Such compounds, as well as pharmaceutically acceptable salts and compositions thereof, are useful for treating diseases or conditions a disease or disorder that can be treated with a KOR agonist and/or MOR agonist. Compounds described herein, along with pharmaceutically acceptable salts and compositions thereof, can be used to alleviate at least one symptom of the disease or disorder described herein.

### INCORPORATION BY REFERENCE TO ANY PRIORITY APPLICATIONS

This application claims priority to U.S. Provisional Patent Application Nos. 63/279,094, filed Nov. 13, 2021; 63/282,264, filed Nov. 23, 2021; 63/299,749, filed Jan. 14, 2022; 63/317,980, filed Mar. 9, 2022; 63/321,915, filed Mar. 21, 2022; 63/336,476, filed Apr. 29, 2022; and 63/375,050, filed Sep. 8, 2022; each of which are hereby incorporated herein by reference to their entireties to the extent not inconsistent with the content of this disclosure.

### BACKGROUND

### Field

The present application generally relates to pharmaceutical compounds. More specifically, compounds that modulate a KOR and/or a MOR receptor are provided.

### Description

Salvinorin A (SalA) is a neoclerodane diterpenoid isolated from the Mexican hallucinogenic plant Saliva divinorum.

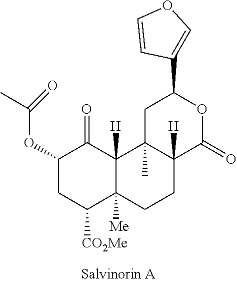

SalA is a potent kappa-opioid receptor (KOR) agonist. Multiple natural products related to SalA have been identified, including collybolide, which is also a potent KOR agonist.

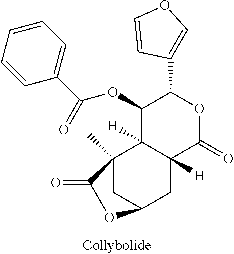 [SMILES: C[C@@]12C[C@@H](C[C@H]3C(=O)O[C@@H](c4ccoc4)[C@H](OC(=O)c4ccccc4)[C@@H]31)OC2=O]

### SUMMARY

Some embodiments provide a compound of Formula I, Formula II, or Formula III, or a pharmaceutically acceptable salt of any of the foregoing.

Some embodiments disclosed herein relate to a pharmaceutical composition that can include an effective amount of one or more compounds of Formula I, Formula II, Formula III, or a pharmaceutically acceptable salt of any of the foregoing, an a pharmaceutically acceptable carrier, diluent, excipient or combination thereof.

Some embodiments described herein relate to a method for treating a subject having a disease or disorder described herein that can include administering an effective amount of a compound described herein (such as a compound of Formula I, Formula II, and/or Formula III, or a pharmaceutically acceptable salt of any of the foregoing) or a pharmaceutical composition that includes a compound described herein (such as a compound of Formula I, Formula II, and/or Formula III, or a pharmaceutically acceptable salt of any of the foregoing) to the subject to alleviate at least one symptom of the disease or disorder (such as 1, 2 or 3 symptoms). Other embodiments described herein relate to the use of an effective amount of a compound described herein (such as a compound of Formula I, Formula II, and/or Formula III, or a pharmaceutically acceptable salt of any of the foregoing) or a pharmaceutical composition that includes a compound described herein (such as a compound of Formula I, Formula II, and/or Formula III, or a pharmaceutically acceptable salt of any of the foregoing) in the manufacture of a medicament for treating a disease or disorder described herein. Still other embodiments described herein relate to an effective amount of a compound described herein (such as a compound of Formula I, Formula II and/or Formula III, or a pharmaceutically acceptable salt of any of the foregoing) or a pharmaceutical composition that includes a compound described herein (such as a compound of Formula I, Formula II, and/or Formula III, or a pharmaceutically acceptable salt of any of the foregoing) for treating a disease or condition described herein.

Some embodiments described herein relate to a method for treating a subject having a disease or disorder described herein that can include contacting a KOR or a MOR receptor in a subject with an effective amount of a compound described herein (such as a compound of Formula I, Formula II, and/or Formula III, or a pharmaceutically acceptable salt of any of the foregoing). Other embodiments described herein relate to the use of an effective amount of a compound described herein (such as a compound of Formula I, Formula II, and/or Formula III, or a pharmaceutically acceptable salt of any of the foregoing) in the manufacture of a medicament for contacting a KOR or a MOR receptor in a subject having a disease or disorder described herein. Still other embodiments described herein relate to an effective amount of a compound described herein (such as a compound of Formula I, Formula II, and/or Formula III, or a pharmaceutically acceptable salt of any of the foregoing) for contacting a KOR or a MOR receptor in a subject having a disease or disorder described herein.

Some embodiments described herein relate to a method for ameliorating one or more symptom (such as 1, 2 or 3 symptoms) of a disease or disorder described herein that can include administering an effective amount of a compound described herein (such as a compound of Formula I, Formula II, and/or Formula III, or a pharmaceutically acceptable salt of any of the foregoing) to a subject suffering from the disease or disorder; and can also include contacting a KOR or a MOR receptor with an effective amount of a compound described herein (such as a compound of Formula I, Formula II, and/or Formula III, or a pharmaceutically acceptable salt of any of the foregoing). Other embodiments described herein relate to the use of an effective amount of a compound described herein (such as a compound of Formula I, Formula II, and/or Formula III, or a pharmaceutically acceptable salt of any of the foregoing) in the manufacture of a medicament for ameliorating one or more symptom (such as 1, 2 or 3 symptoms) of a disease or disorder described herein; or, in the manufacture of a medicament for ameliorating one or more symptom (such as 1, 2 or 3 symptoms) of a disease or disorder described herein, wherein the use comprises contacting a KOR or a MOR receptor with the medicament. Still other embodiments described herein relate to an effective amount of a compound described herein (such as a compound of Formula I, Formula II, and/or Formula III, or a pharmaceutically acceptable salt of any of the foregoing) for ameliorating one or more symptom (such as 1, 2 or 3 symptoms) of a disease or disorder described herein by contacting a KOR or a MOR receptor.

These and other embodiments are described in greater detail below.

### BRIEF DESCRIPTION OF THE DRAWINGS

FIGS. 1A and 1B depict a flow chart for compound testing.

FIGS. 2A and 2B depict steps for total synthesis of racemic O6C-20-nor-SalA.

FIG. 3 depicts a method for preparation of per-deuterated-acetyl SalA.

FIG. 4 depicts a method for synthesizing O6C-20-nor-SalB.

FIG. 5A depicts a synthetic route for preparing O-acylated compounds.

FIGS. 5B and 5C show synthetic routes for various embodiments of O-acylated compounds.

FIG. 5D depicts a proposed method for synthesizing C2-epi-O-acylated compounds.

FIGS. 6A and 6B depict a method for O-alkylation or O-arylation of the shown compounds.

FIG. 7A depicts a proposed method for N-acylation of the shown multicyclic compounds.

FIGS. 7B and 7C show synthetic routes for various embodiments of N-acylated multicyclic compounds.

FIG. 8 depicts a second proposed method for N-acylation of the shown multicyclic compounds.

FIGS. 9A and 9B depict methods for synthesizing a multicyclic compound wherein the stereochemistry of the furyl group has been changed.

FIG. 10 depicts a proposed synthesis of the shown multicyclic compounds.

FIG. 11 depicts a proposed synthesis of the shown multicyclic compounds.

### DETAILED DESCRIPTION

### Compounds

Various embodiments disclosed herein relate to a compound of Formula I, Formula II, or Formula III, or a pharmaceutically acceptable salt of any of the foregoing, having the structures:

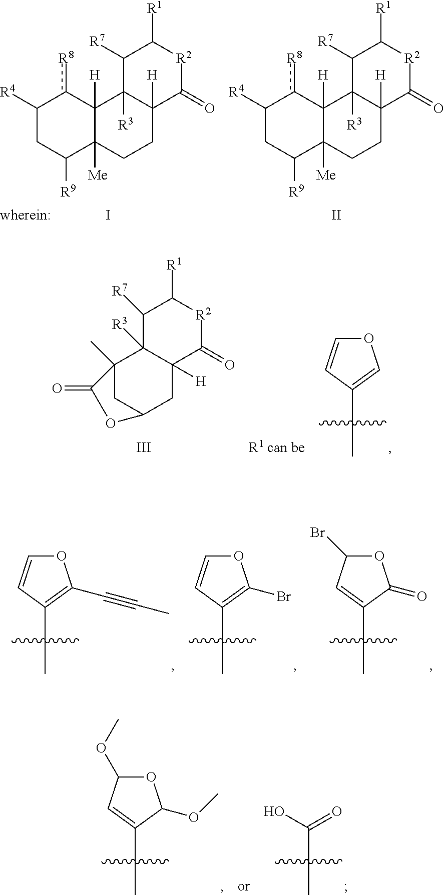

R² can be O or CH₂;
R³ can be H or CH₃;

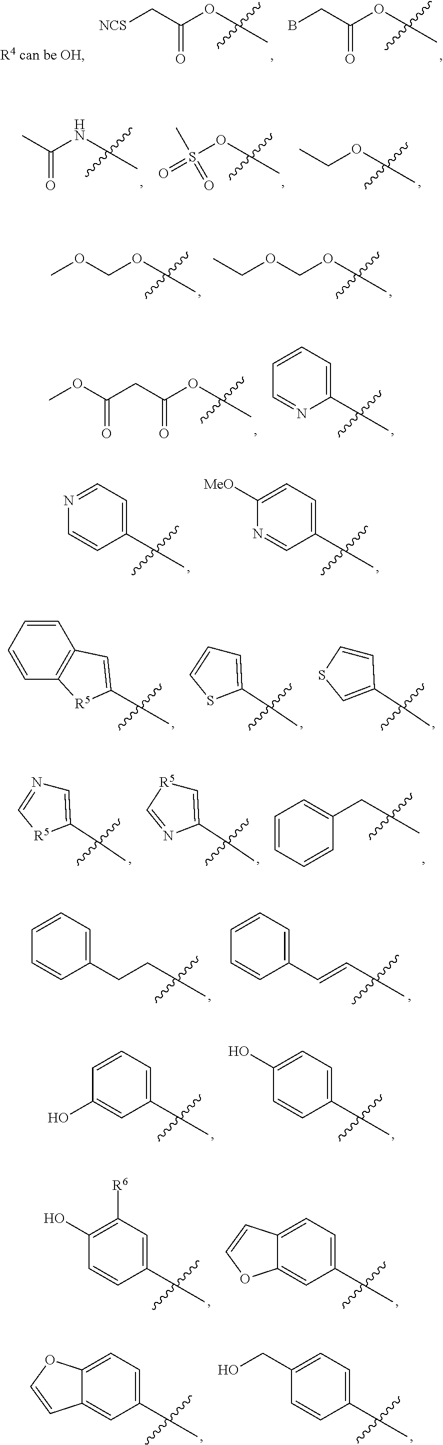

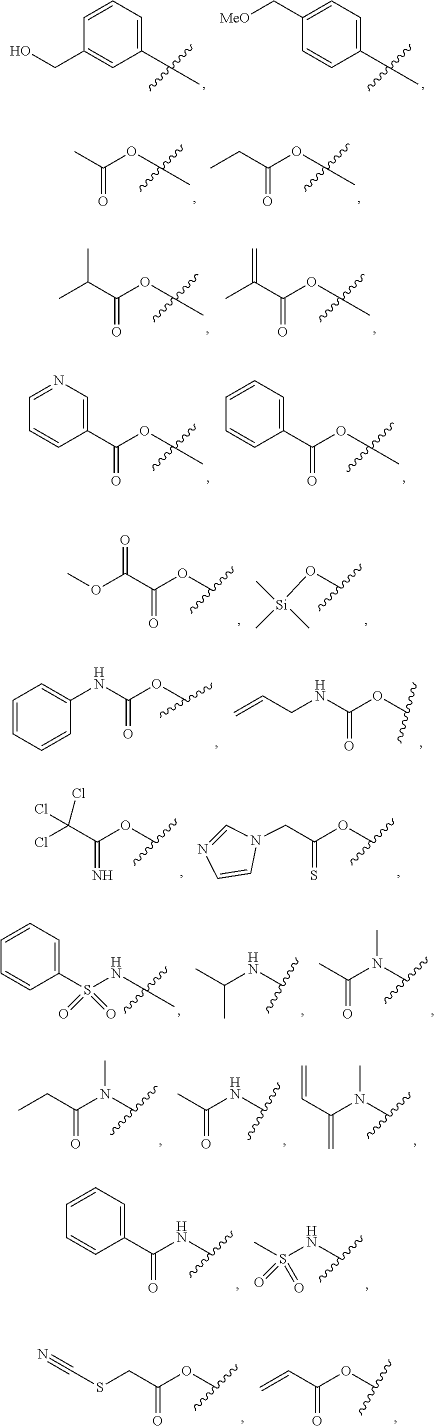

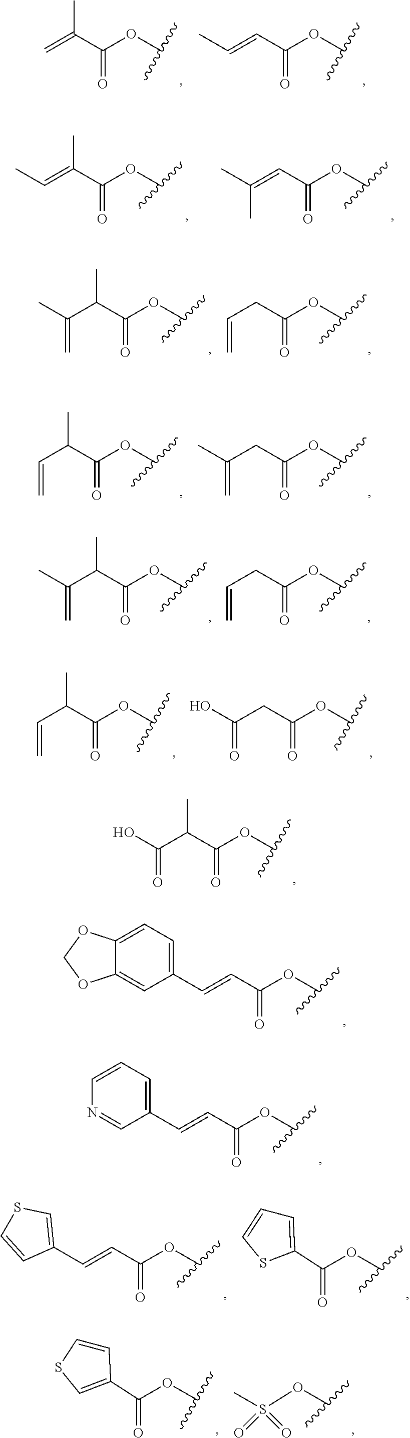

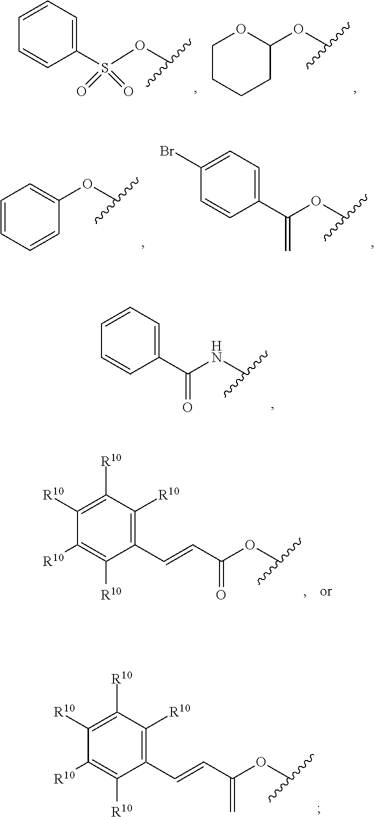

wherein R⁵ can be O or S;
   R⁶ can be F, Cl, or Br; and
   each R¹⁰ can independently be H, OMe, NO₂, or CF₃;
R⁷ can be H or

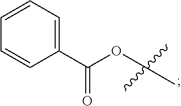 [SMILES: *OC(=O)c1ccccc1]

R⁸ can be O, OH, or

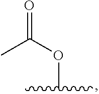 [SMILES: *OC(C)=O]

and [symbol] represents a single bond or a double bond, wherein
when R⁸ is O, the bond represented by [symbol] is a double bond and when R⁸ is OH or

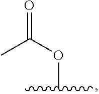 [SMILES: *OC(C)=O]

the bond represented by [symbol] is a single bond;
R⁹ can be CO₂Me or CONH₂;
and NCS is N-chlorosuccinimide;
provided that one or both of the following conditions is met: R² is CH₂, or R³ is H; and
provided that the compound is not selected from:

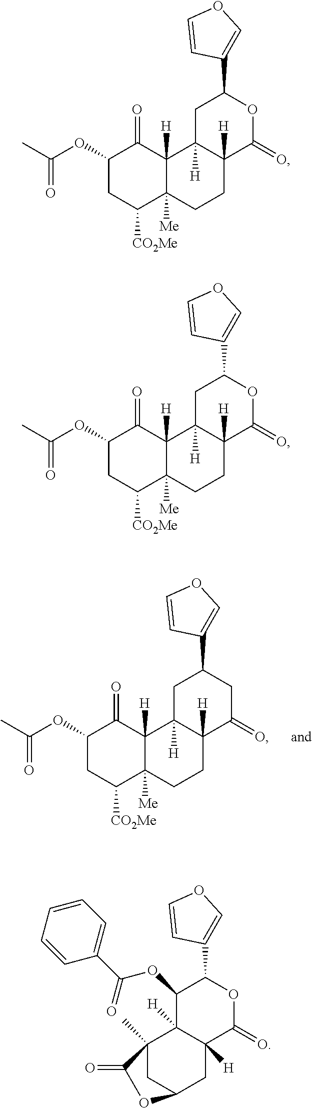

As used herein, “ [symbol] ” crosses a bond that is covalently bound to Formula I, Formula II, or Formula III at an R group. In some embodiments, to Formula I, Formula II, or Formula III, or a pharmaceutically acceptable salt of any of the foregoing, cannot be

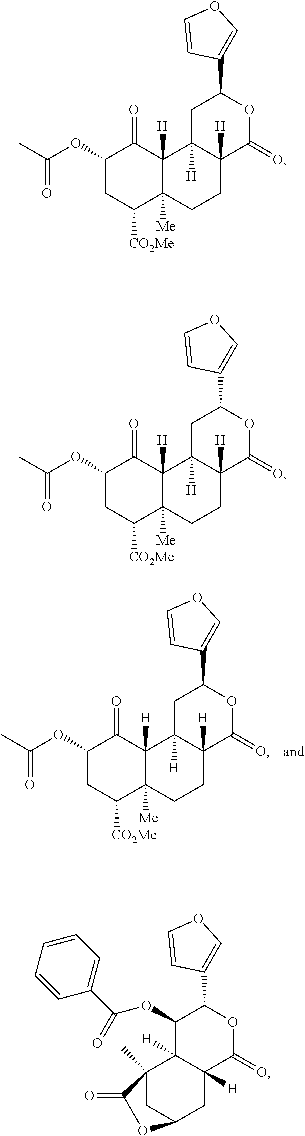

or a pharmaceutically acceptable salt of any of the foregoing.

As used herein, “ [symbol] ” represents a bond that may be a single bond or a double bond; wherein when R⁸ is O, “ [symbol] ” represents a double bond to the oxygen and when R⁸ is

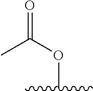 [SMILES: *OC(C)=O]

or OH, “ [symbol] ” represents a single bond to the oxygen of

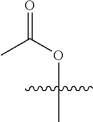 [SMILES: *OC(C)=O]

### or OH.

In some embodiments, the compound of Formula I, or a pharmaceutically acceptable salt thereof, can be represented by Formula I.a:

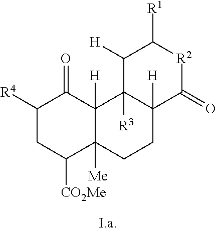

In some embodiments of Formula I.a, or a pharmaceutically acceptable salt thereof, R¹ can be

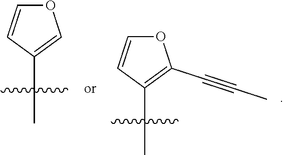

In some embodiments of Formula I.a, or a pharmaceutically acceptable salt thereof, R² can be O or CH₂.

In some embodiments of Formula I.a, or a pharmaceutically acceptable salt thereof, R³ can be H or CH₃. Those skilled in the art understand that CH₃ can be represented as “Me.”

In some embodiments of Formula I.a, or a pharmaceutically acceptable salt thereof, R⁴ can include a halogen, such as

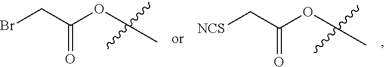

wherein NCS is N-chlorosuccinimide.

In some embodiments of Formula I.a, or a pharmaceutically acceptable salt thereof, R¹ can be can be

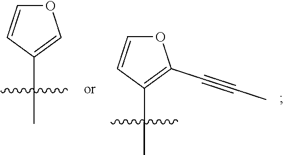

R² can be O or CH₂; R³ can be H or CH₃; and R⁴ can include a halogen, such as

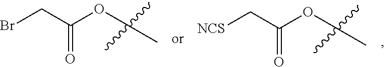

wherein NCS is N-chlorosuccinimide. In some embodiments, the compound of Formula I.a, or a pharmaceutically acceptable salt thereof, can be selected from:

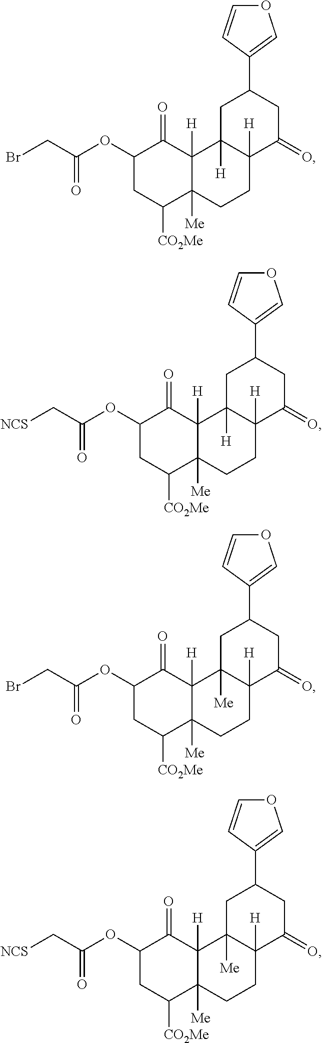

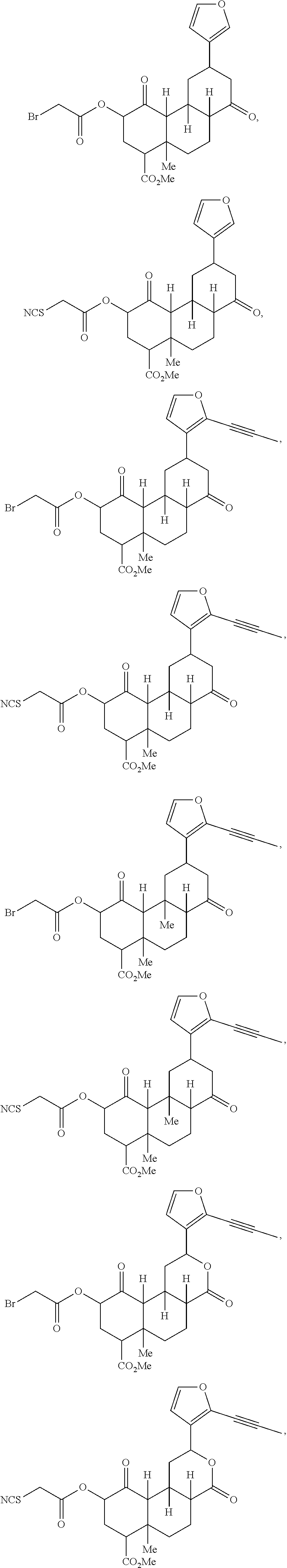

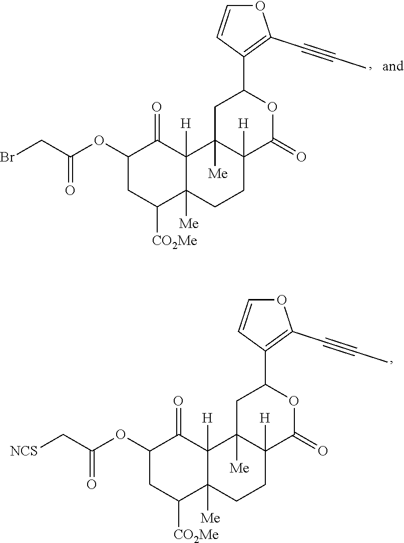

(including pharmaceutically acceptable salts of any of the foregoing).

In other embodiments of Formula I.a, or a pharmaceutically acceptable salt thereof, R⁴ cannot include a double bond. Exemplary R⁴ moieties that do not include a double bond are OH,

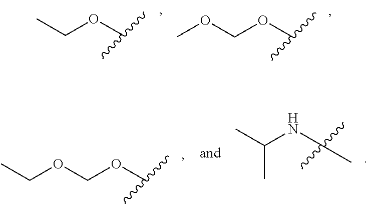

In some embodiments of Formula I.a, or a pharmaceutically acceptable salt thereof, R¹ can be

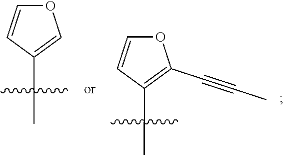

R² can be O or CH₂; R³ can be H or CH₃; and R⁴ cannot include a double bond. In some embodiments, the compound of Formula I.a, or a pharmaceutically acceptable salt thereof, can be selected from:

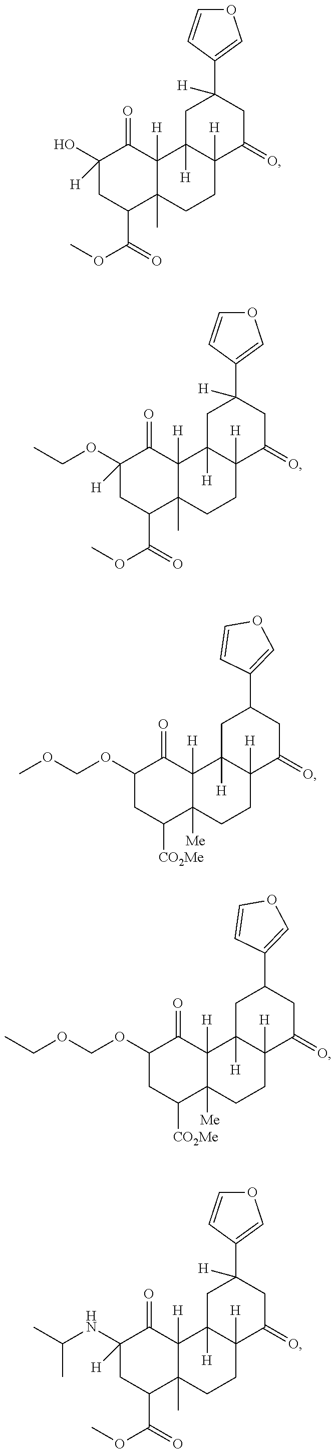

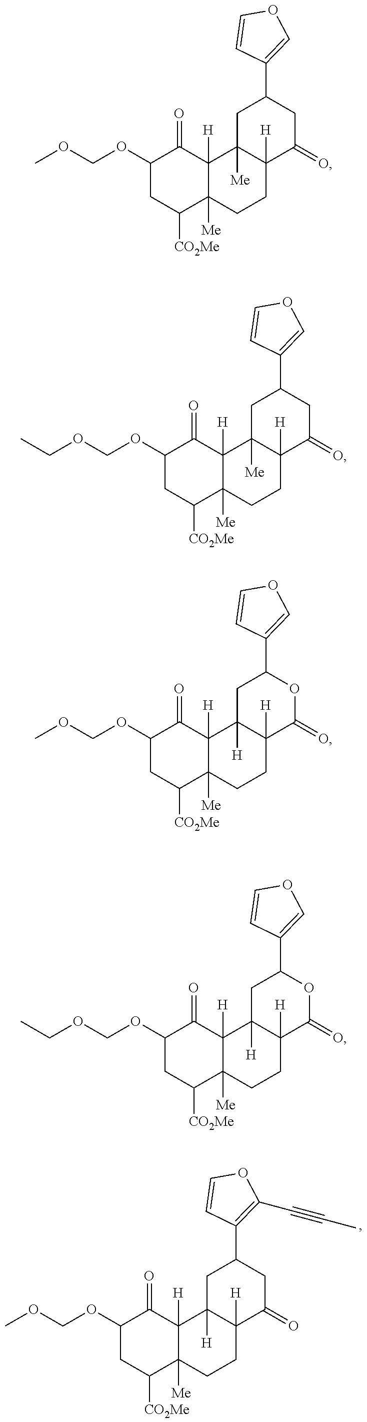

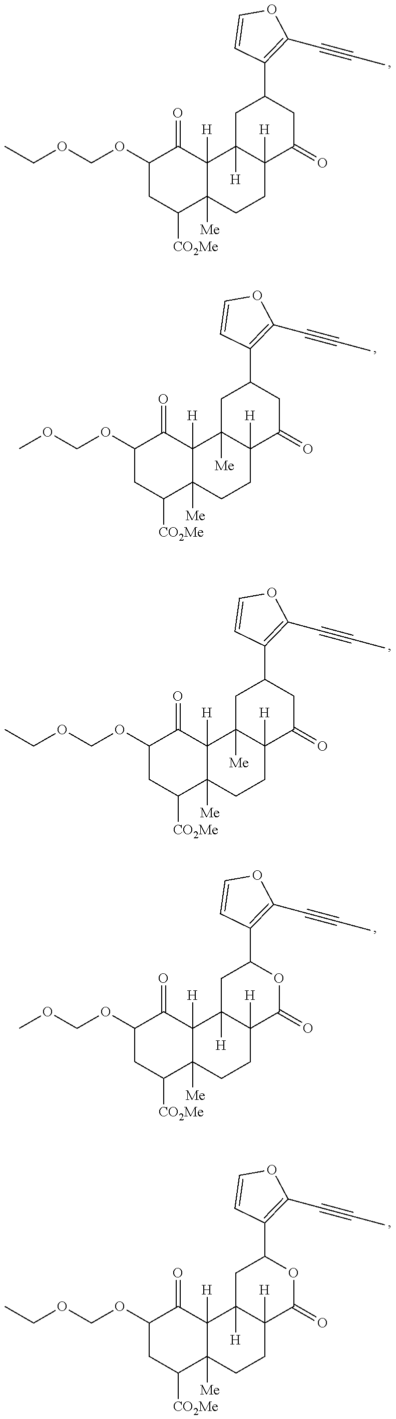

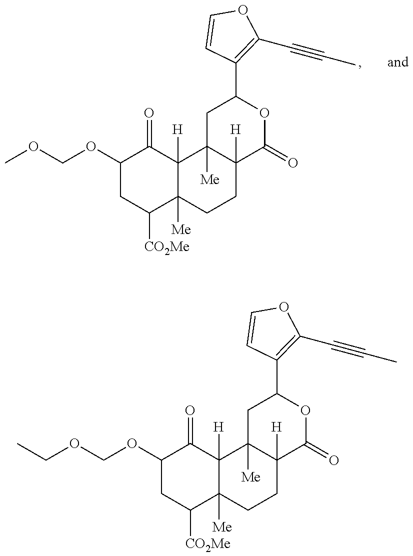

(including pharmaceutically acceptable salts of any of the foregoing).

In other embodiments of Formula I.a, or a pharmaceutically acceptable salt thereof, R⁴ can include an aromatic ring, such as

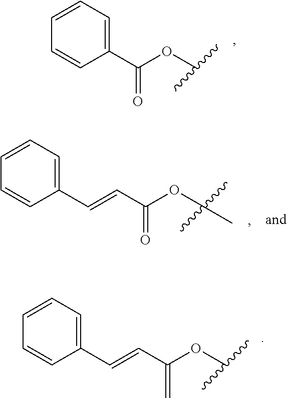 [SMILES: *OC(=O)c1ccccc1 ; *OC(=O)/C=C/c1ccccc1 ; *OC(=C)/C=C/c1ccccc1]

In some embodiments of Formula I.a, or a pharmaceutically acceptable salt thereof, R¹ can be or

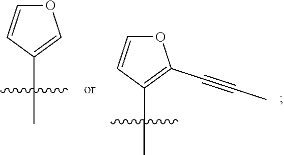

R² can be O or CH₂, R³ can be H or CH₃; and R⁴ can include an aromatic ring, such as

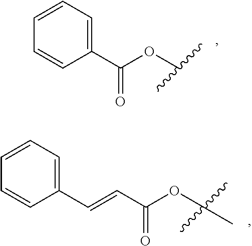 [SMILES: *OC(=O)c1ccccc1 ; *OC(=O)/C=C/c1ccccc1]

and

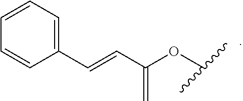 [SMILES: *OC(=C)/C=C/c1ccccc1]

In some embodiments, the compound of Formula I.a, or a pharmaceutically acceptable salt thereof, can be selected from:

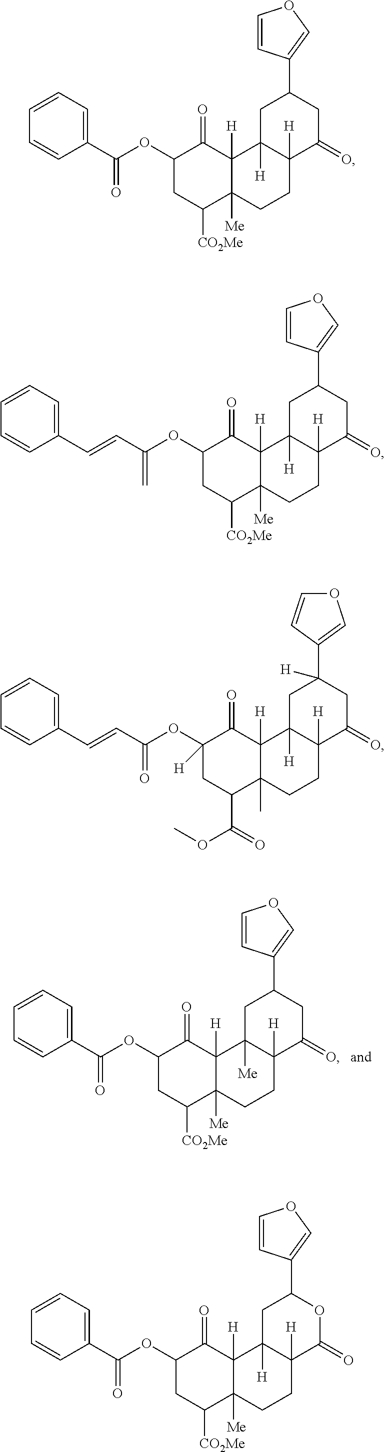

(including pharmaceutically acceptable salts of any of the foregoing).

In other embodiments of Formula I.a, or a pharmaceutically acceptable salt thereof, R⁴ can comprise at least one double bond (such as 1 or 2 double bonds) and not comprise an aromatic ring, N (nitrogen), or S (sulfur), such as

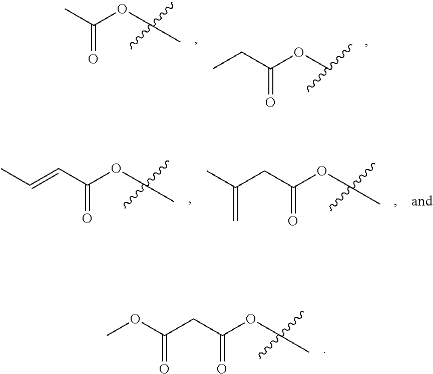

In some embodiments of Formula I.a, or a pharmaceutically acceptable salt thereof, R¹ can be

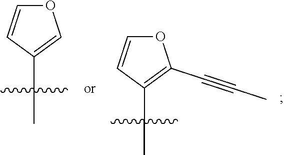

R² can be O or CH₂; R³ can be H or CH₃; and R⁴ can comprise at least one double bond (such as 1 or 2 double bonds) and not comprise an aromatic ring, N, or S, such as

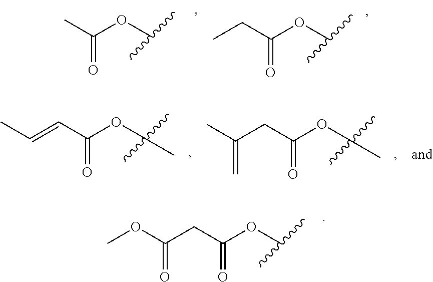

In some embodiments, the compound of Formula I.a, or a pharmaceutically acceptable salt thereof, can be selected from:

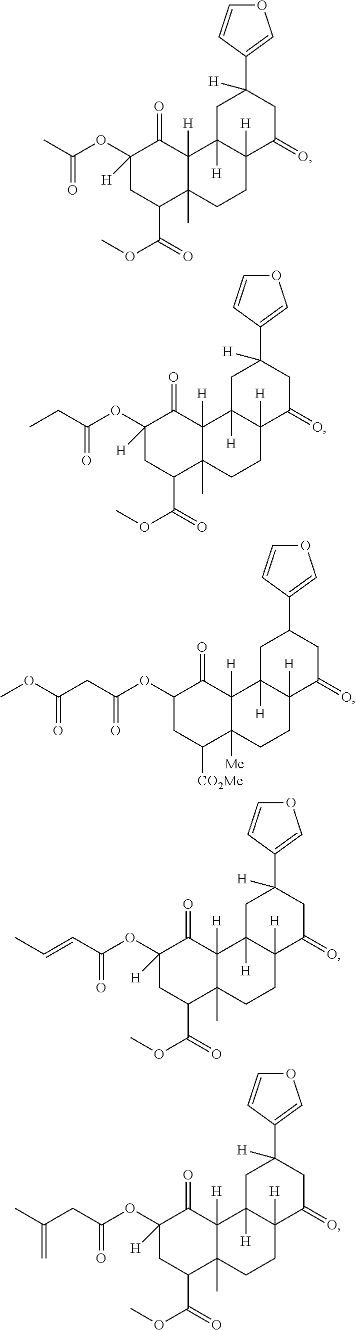

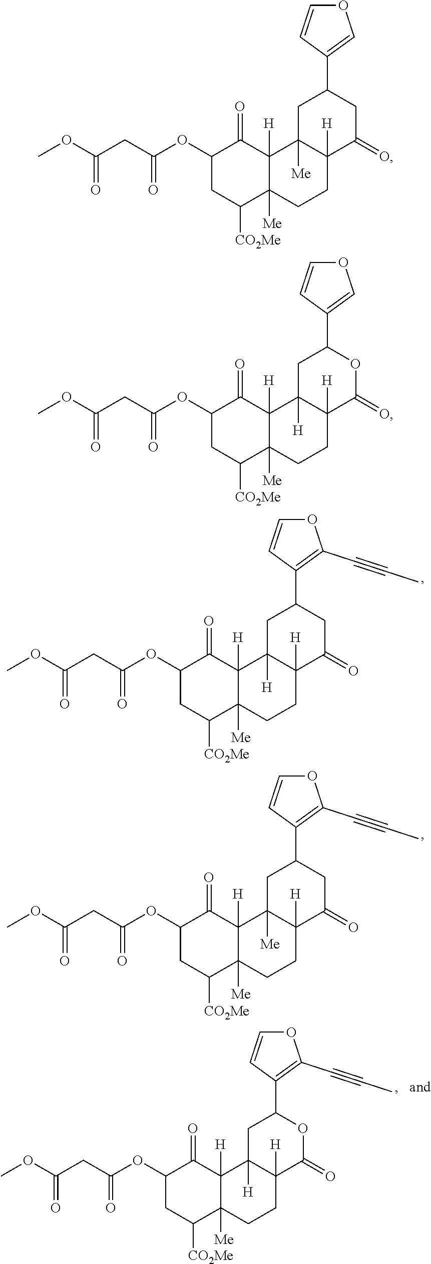

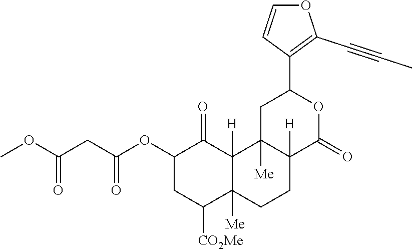

(and including pharmaceutically acceptable salts of any of the foregoing).

In other embodiments of Formula I.a, or a pharmaceutically acceptable salt thereof, R⁴ can include an O (oxygen). Exemplary R⁴ moieties that include an O are:

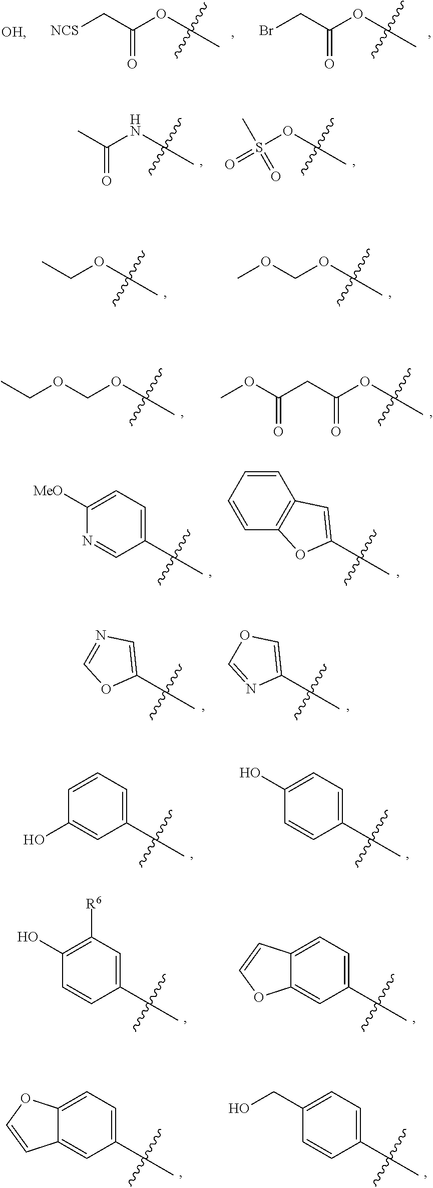

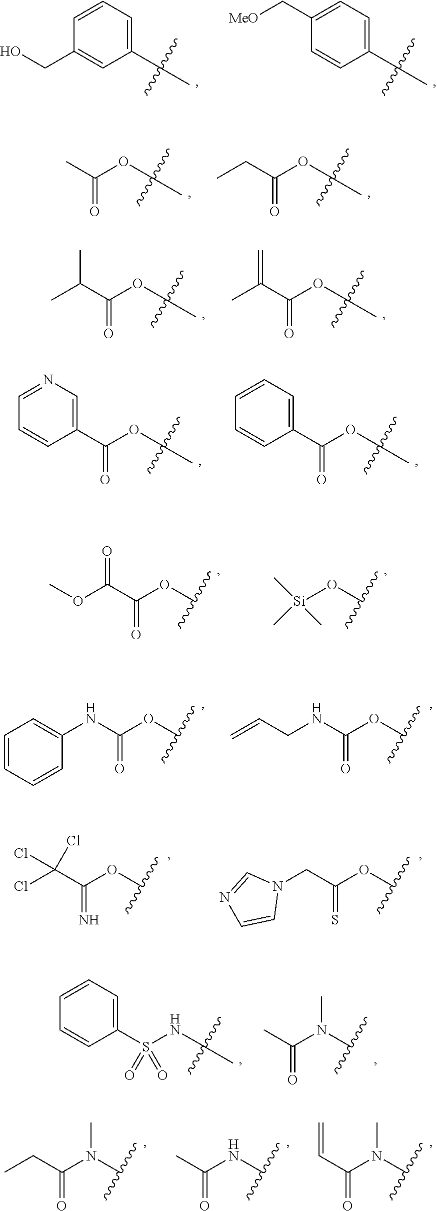

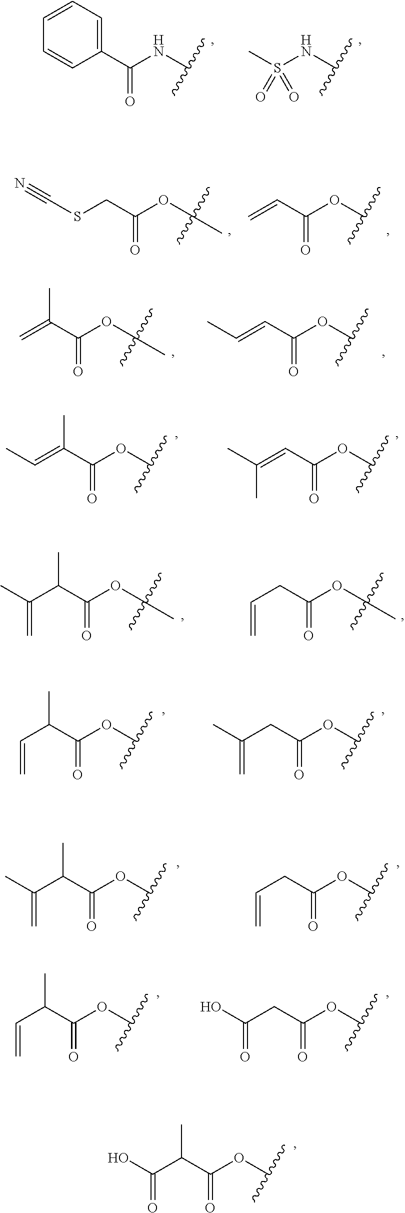

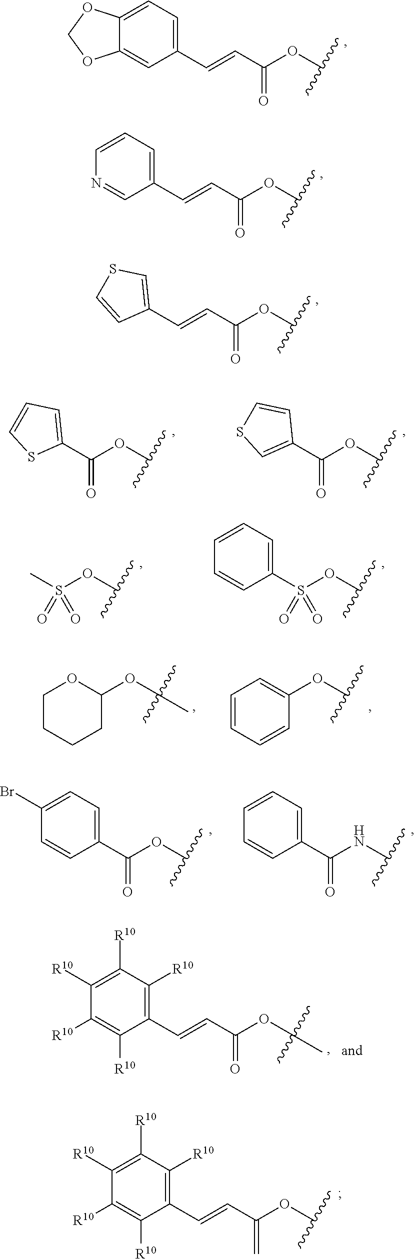

wherein R⁶ can be F, Cl, or Br; and
each R¹⁰ can independently be H, OMe, NO₂, or CF.

In some embodiments of Formula I.a, or a pharmaceutically acceptable salt thereof, R¹ can be

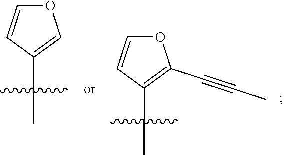

R² can be O or CH₂; R³ can be H or CH₃; and R⁴ can include an O. In some embodiments, the compound of Formula I.a, or a pharmaceutically acceptable salt thereof, can be selected from:

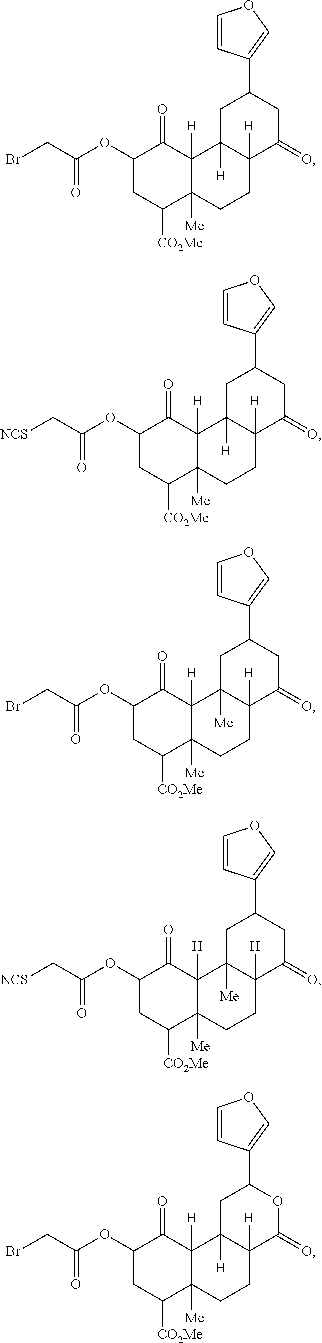

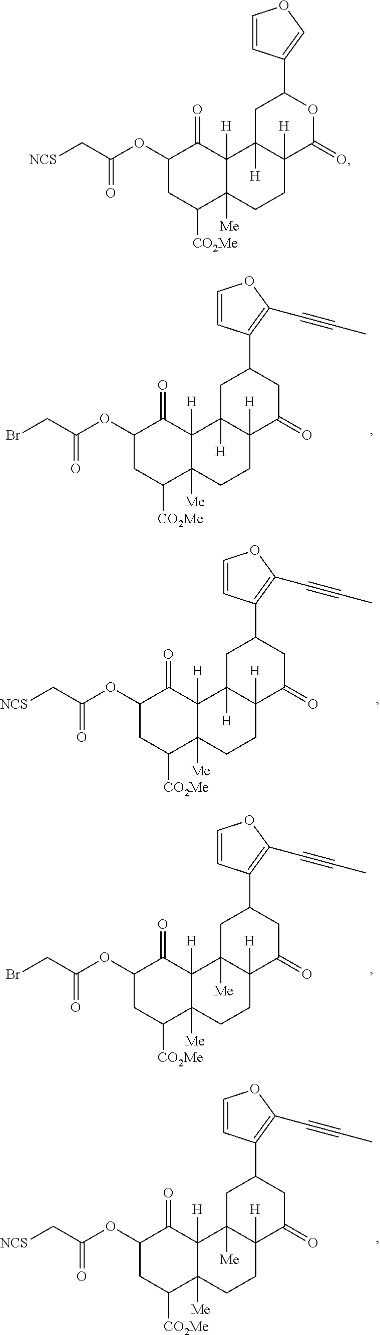

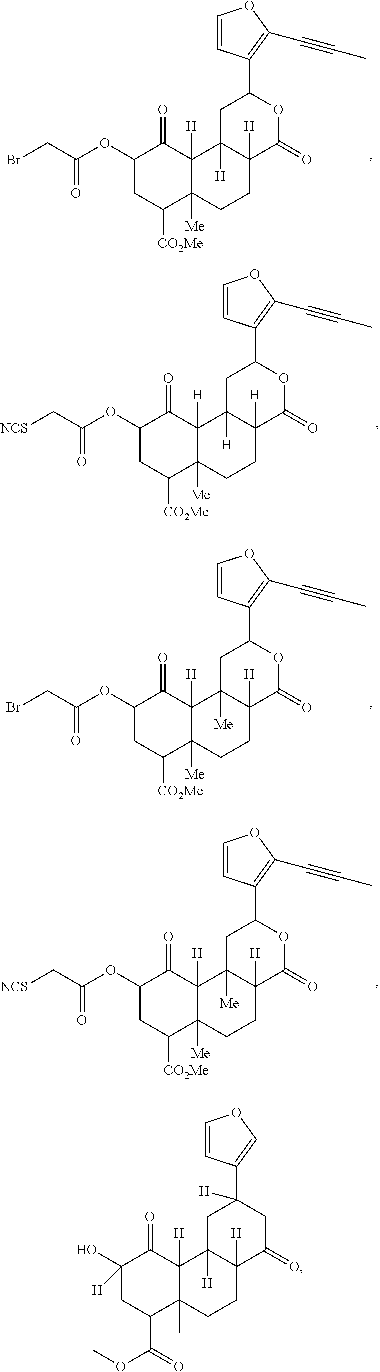

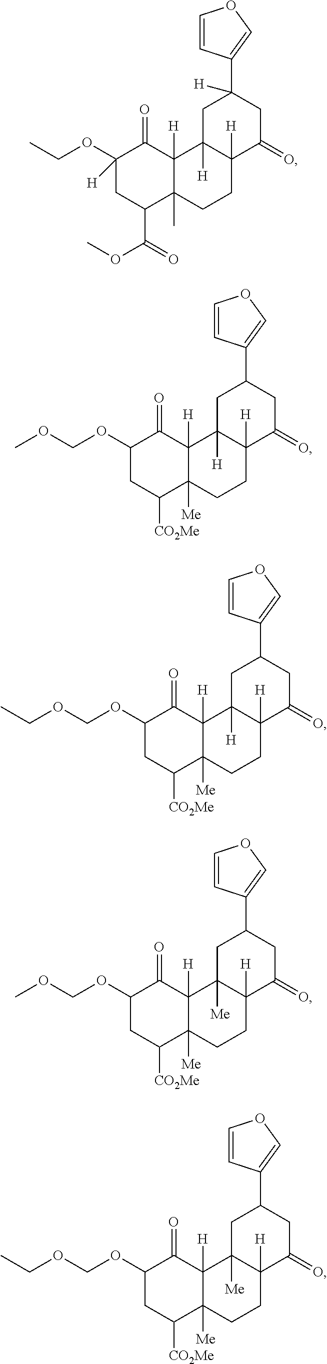

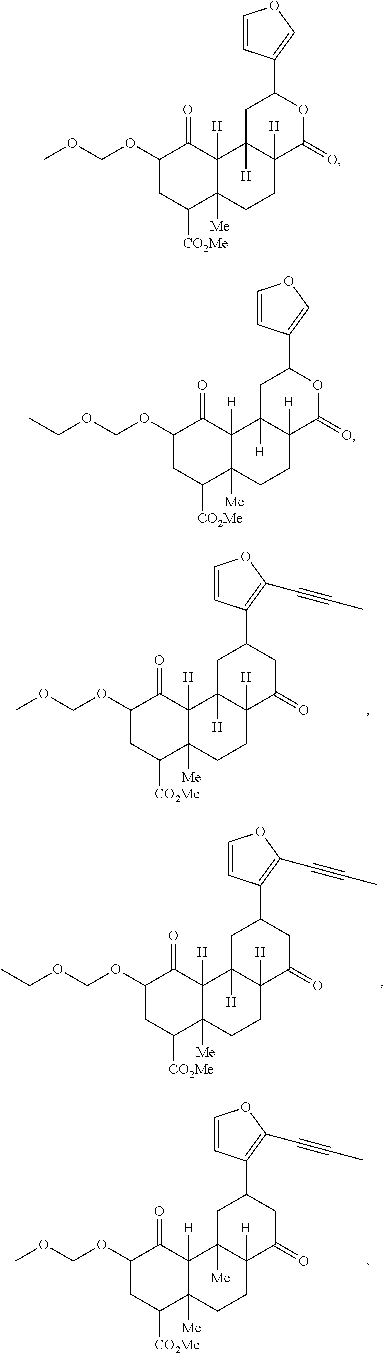

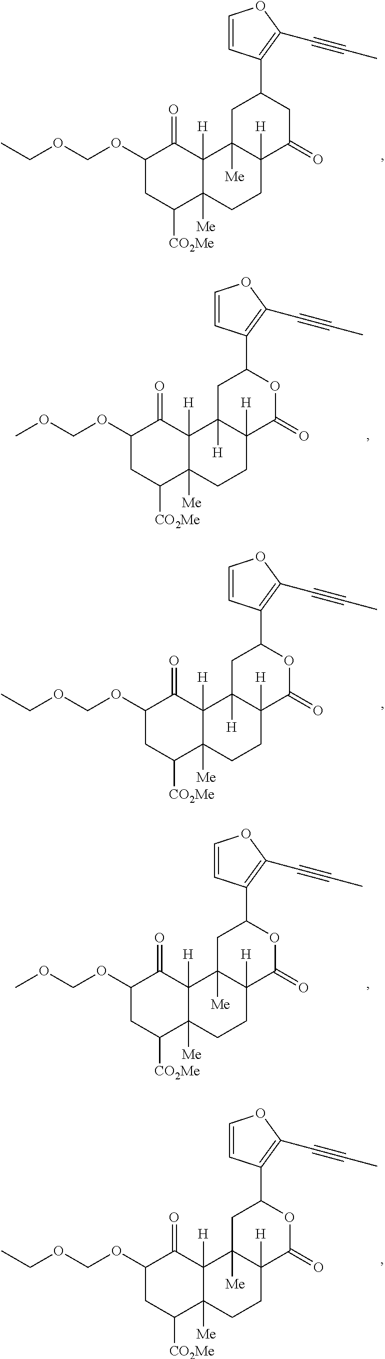

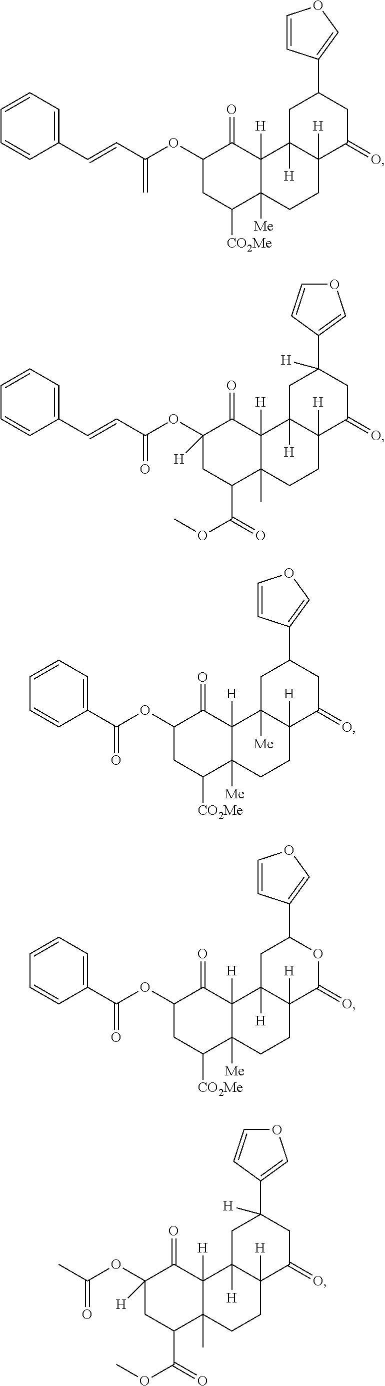

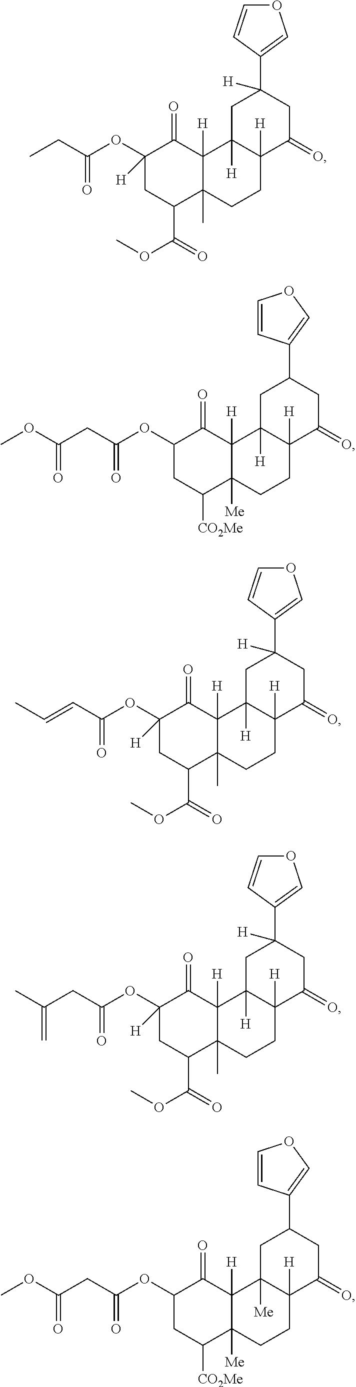

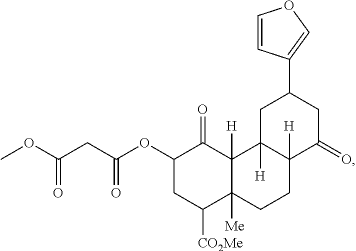

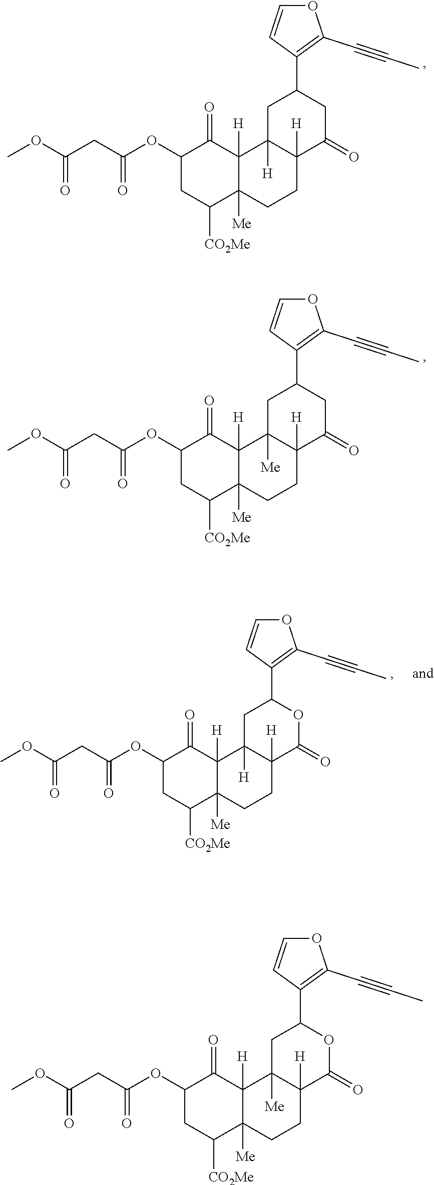

(including pharmaceutically acceptable salts of any of the foregoing). In some embodiments, the compound of Formula I.a, or a pharmaceutically acceptable salt thereof, can be selected from:

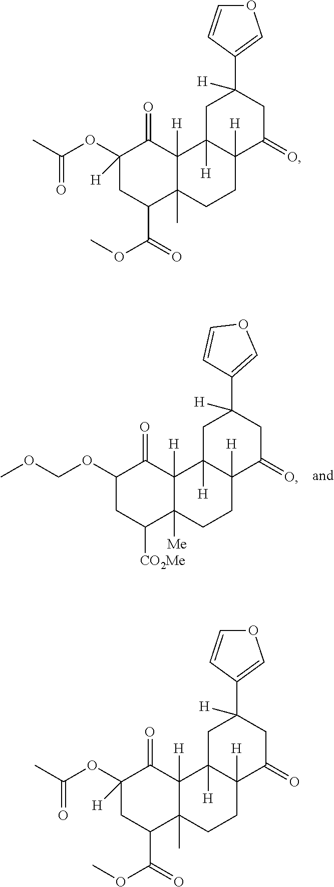

including pharmaceutically acceptable salts of any of the foregoing).

In other embodiments of Formula I.a, or a pharmaceutically acceptable salt thereof, R⁴ can comprise at least one double bond (such as 1 or 2 double bonds) and at least one atom selected from N (nitrogen) and S (sulfur) (such as 1 nitrogen, 1 sulfur, 2 nitrogens, 2 sulfurs, or 1 nitrogen and 1 sulfur), for example,

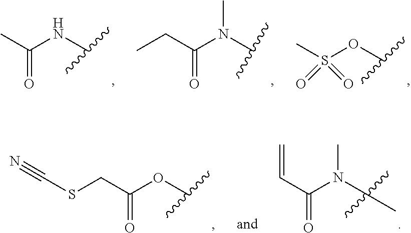

In some embodiments of Formula I.a, or a pharmaceutically acceptable salt thereof, R¹ can be

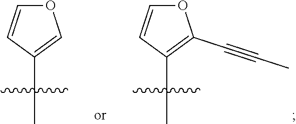

R² can be O or CH₂; R¹ can be H or CH₃; and R⁴ can comprise at least one double bond (such as 1 or 2 double bonds) and at least one atom selected from N and S (such as 1 nitrogen, 1 sulfur, 2 nitrogens, 2 sulfurs, or 1 nitrogen and 1 sulfur), for example,

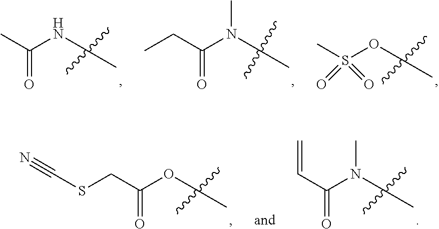

In some embodiments, the compound of Formula I.a, or a pharmaceutically acceptable salt thereof, can be selected from:

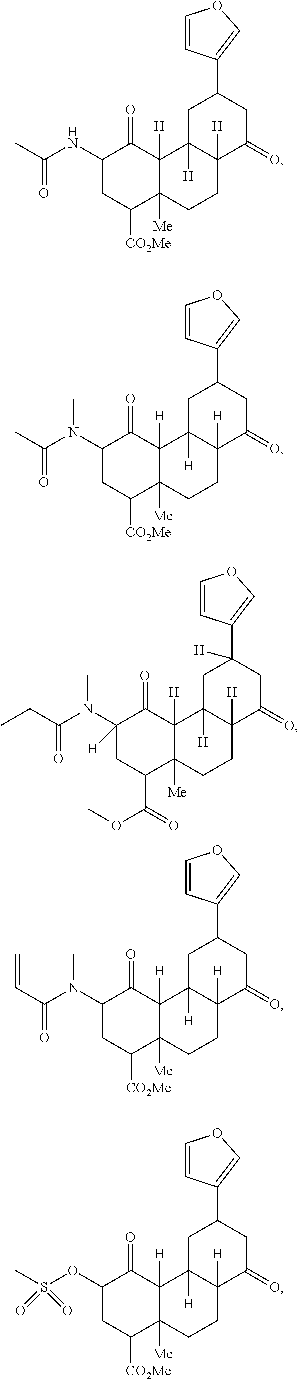

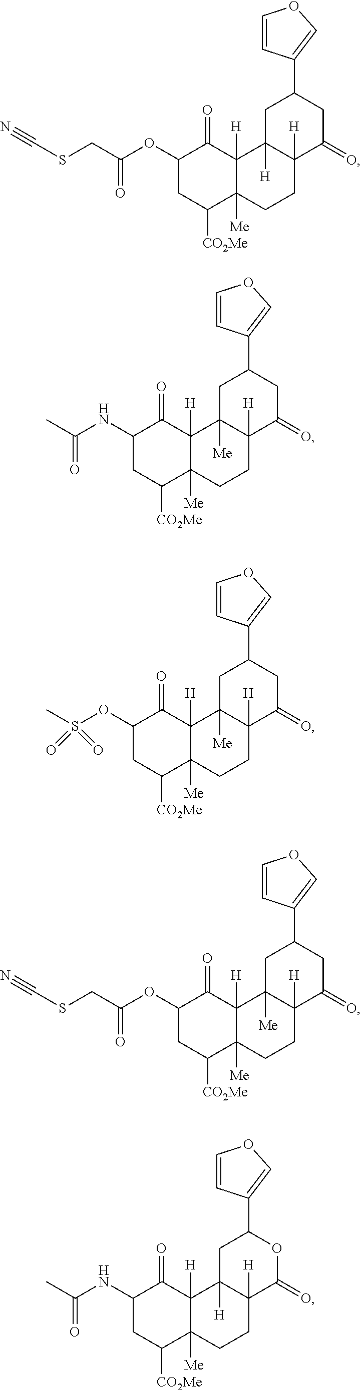

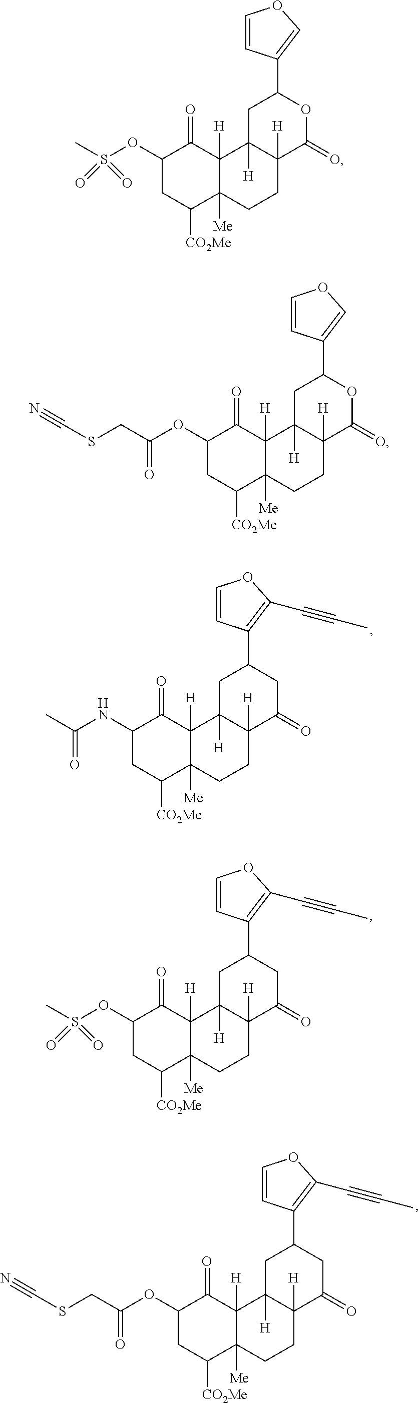

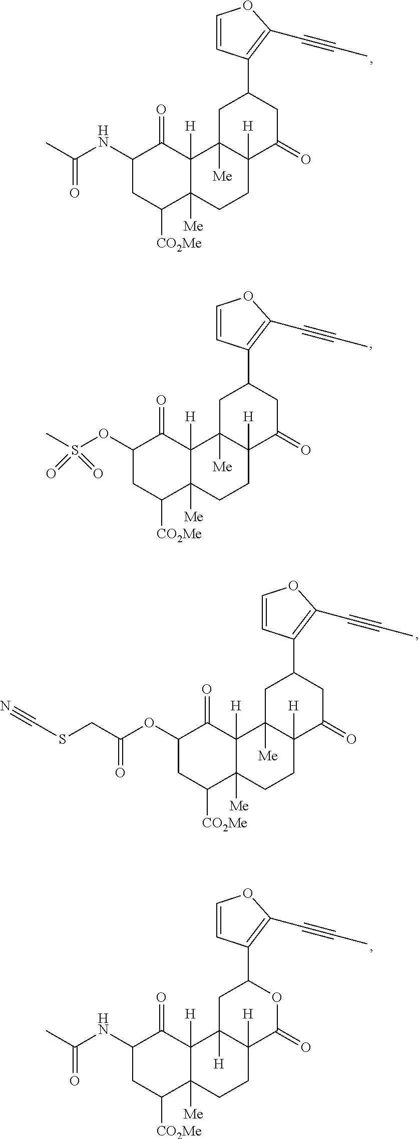

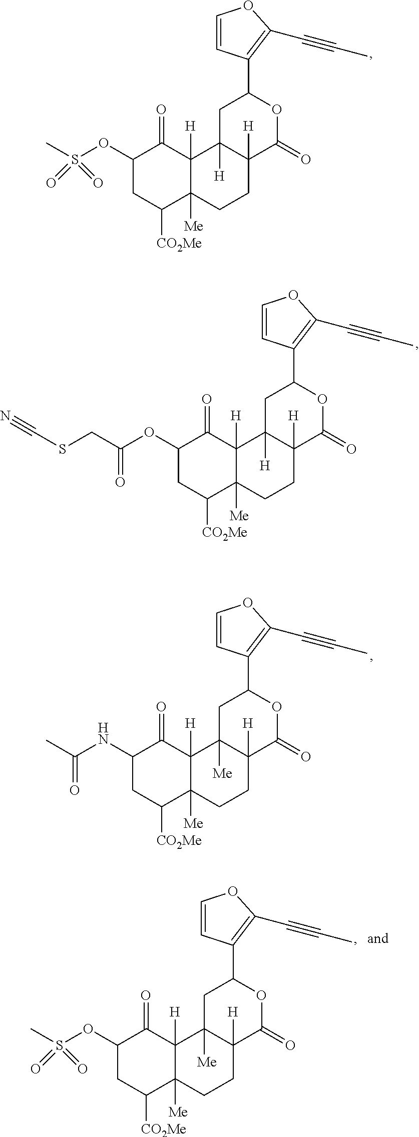

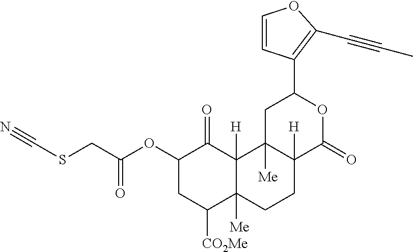

(including pharmaceutically acceptable salts of any of the foregoing).

In other embodiments of Formula I.a, or a pharmaceutically acceptable salt thereof, the compound of Formula I.a, or a pharmaceutically acceptable salt thereof, cannot include

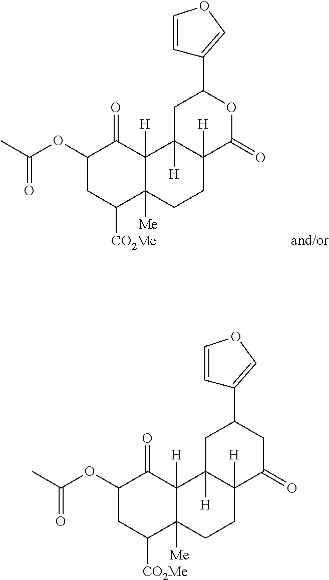

(including pharmaceutically acceptable salts of any of the foregoing).

In some embodiments, the compound of Formula II, or a pharmaceutically acceptable salt thereof, can be represented by Formula II.a:

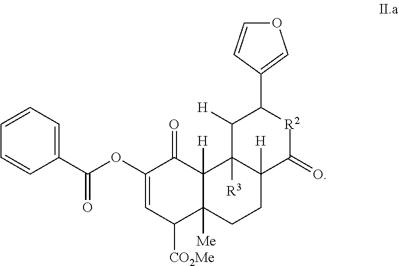

In some embodiments of Formula II.a, or a pharmaceutically acceptable salt thereof, R² can be O or CH₂.

In some embodiments of Formula II.a, or a pharmaceutically acceptable salt thereof, R³ can be H or CH₃.

In some embodiments of Formula II.a, or a pharmaceutically acceptable salt thereof, R² can be O or CH₂ and R³ can be H or CH₃. In some embodiments, the compound of Formula II.a, or a pharmaceutically acceptable salt thereof, can be selected from:

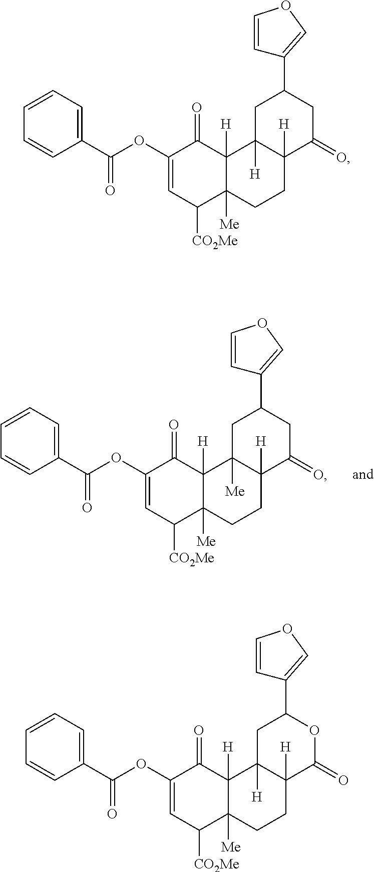

(including pharmaceutically acceptable salts of any of the foregoing).

In some embodiments, the compound of Formula III, or a pharmaceutically acceptable salt thereof, can be represented by Formula III.a:

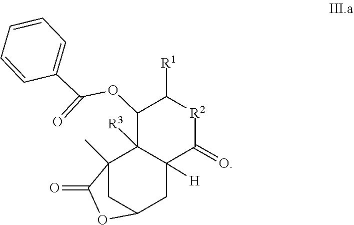

In some embodiments of Formula III.a, or a pharmaceutically acceptable salt thereof, R¹ can be

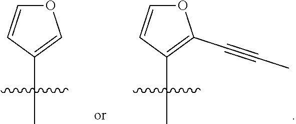

In some embodiments of Formula II.a, or a pharmaceutically acceptable salt thereof, R² can be O or CH₂.

In some embodiments of Formula III.a, or a pharmaceutically acceptable salt thereof, R³ can be H or CH₃.

In some embodiments of Formula III.a, or a pharmaceutically acceptable salt thereof, R¹ can be

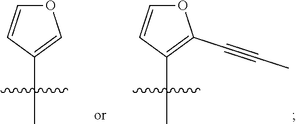

R² can be O or CH₂, and R³ can be H or CH₃. In some embodiments, the compound of Formula II.a, or a pharmaceutically acceptable salt thereof, can be selected from:

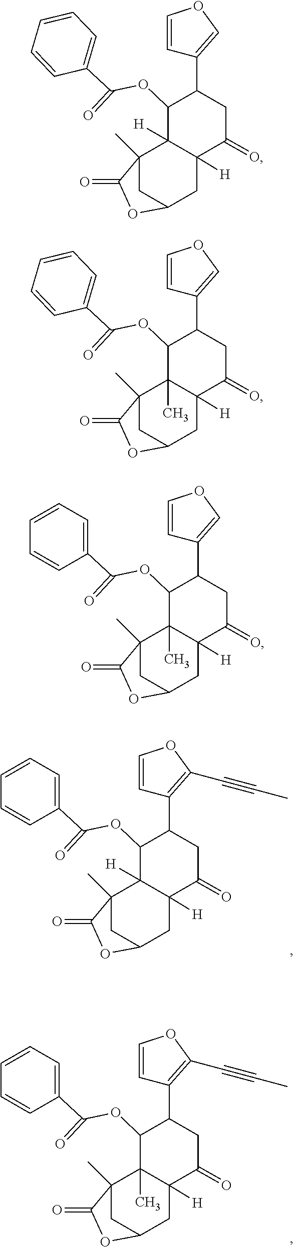 [SMILES: CC12CC(CC3C(=O)CC(c4ccoc4)C(OC(=O)c4ccccc4)C31)OC2=O ; CC12CC(CC3C(=O)CC(c4ccoc4)C(OC(=O)c4ccccc4)C31C)OC2=O ; CC12CC(CC3C(=O)CC(c4ccoc4)C(OC(=O)c4ccccc4)C31C)OC2=O ; CC#Cc1occc1C1CC(=O)C2CC3CC(C)(C(=O)O3)C2C1OC(=O)c1ccccc1 ; CC#Cc1occc1C1CC(=O)C2CC3CC(C)(C(=O)O3)C2(C)C1OC(=O)c1ccccc1]

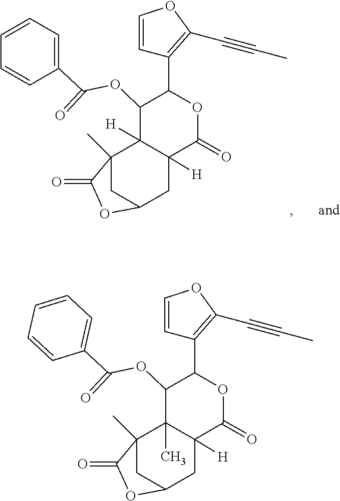 [SMILES: CC#Cc1occc1C1OC(=O)C2CC3CC(C)(C(=O)O3)C2C1OC(=O)c1ccccc1 ; CC#Cc1occc1C1OC(=O)C2CC3CC(C)(C(=O)O3)C2(C)C1OC(=O)c1ccccc1]

(including pharmaceutically acceptable salts of any of the foregoing).

In other embodiments of Formula III.a, or a pharmaceutically acceptable salt thereof, the compound of Formula III.a cannot include

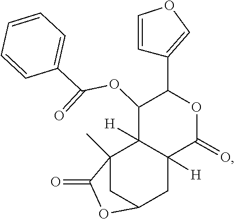 [SMILES: CC12CC(CC3C(=O)OC(c4ccoc4)C(OC(=O)c4ccccc4)C31)OC2=O]

or a pharmaceutically acceptable salt thereof.

Those skilled in the art understand that compounds of Formulae I, II, and III, including pharmaceutically acceptable salts thereof, can have one or more stereocenters.

In some embodiments, the compound of Formula I or Formula III, or a pharmaceutically acceptable salt thereof, can have the structure of Formula I.c or Formula II.c:

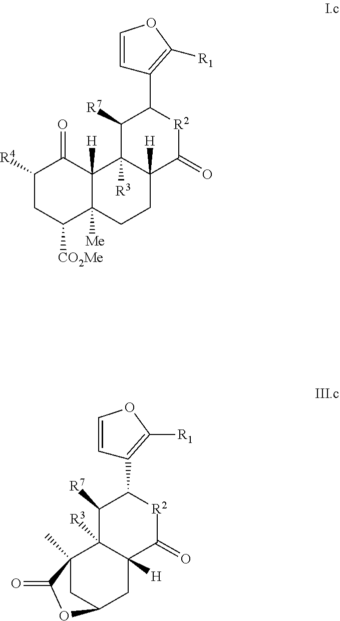

wherein
R¹ can be H or [symbol] ;
R² can be O or CH₂;
R³ can be H or CH₃;
R⁷ can be H or

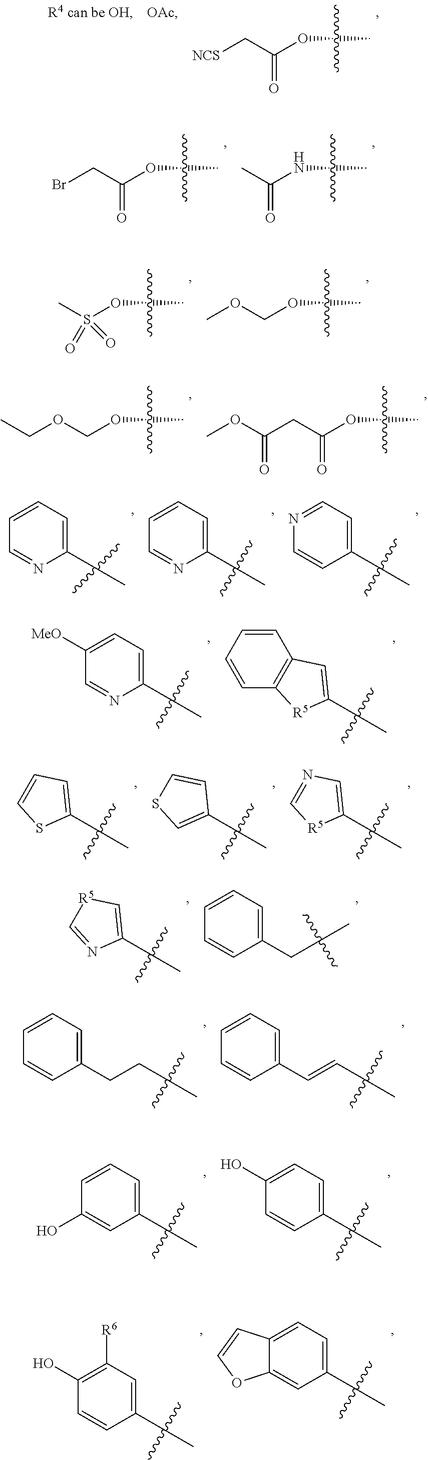

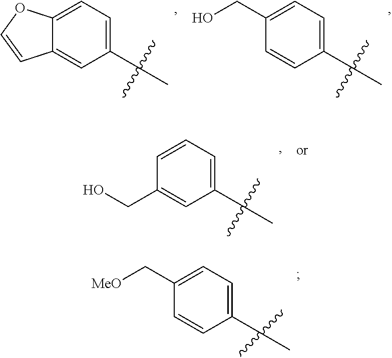 [SMILES: *c1ccc2occc2c1 ; *c1ccc(CO)cc1 ; *c1cccc(CO)c1 ; *c1ccc(COC)cc1]

wherein R⁵ can be O or S; R⁶ can be F, Cl or Br; NCS is N-chlorosuccinimide; and OAc is

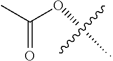

In some embodiments, the compound of Formula I, Formula II, or Formula III, or a pharmaceutically acceptable salt thereof, can have the structure of Formula I.d, Formula II.d, or Formula III.d:

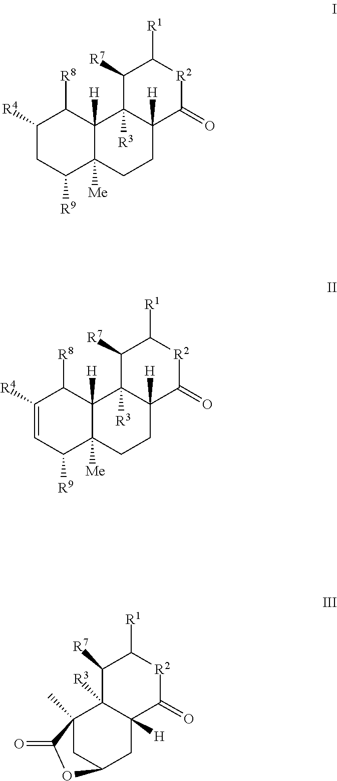

wherein
R¹ can be

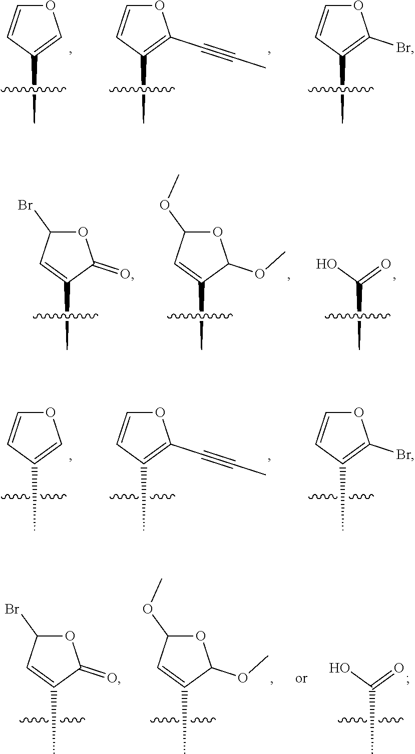

R² can be O or CH₂,
R can be H or CH₃;
R³ can be OH, OAc,

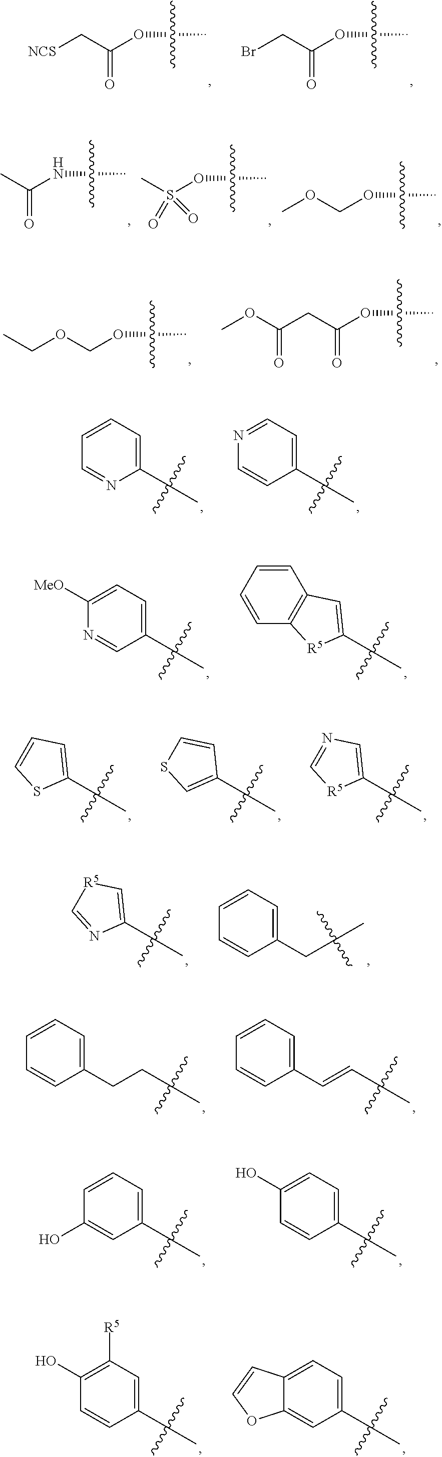

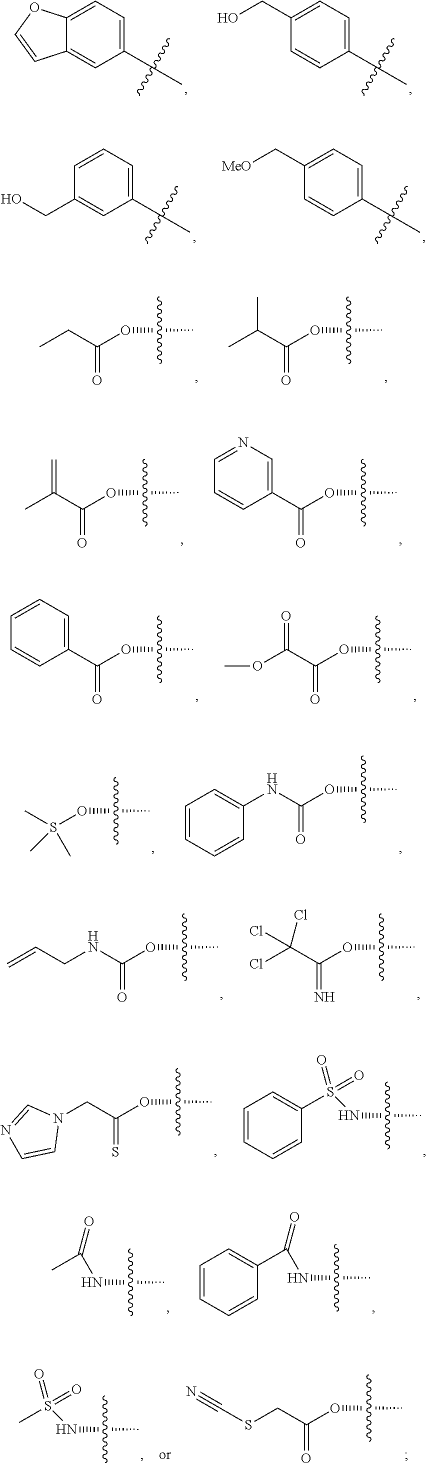

wherein R⁵ can be O or S; and R⁶ can be F, Cl or Br;
R⁷ can be H or

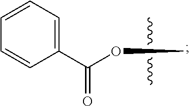

R⁸ can be

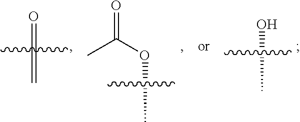

R⁹ can be CO₂Me or CONH₂; and
NCS is N-chlorosuccinimide; and OAc is

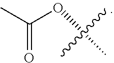

In some embodiments, the compound of Formula I, Formula II, or Formula III, or a pharmaceutically acceptable salt of any of the foregoing, can have the structure of Formula I.e, Formula II.e, or Formula III.e:

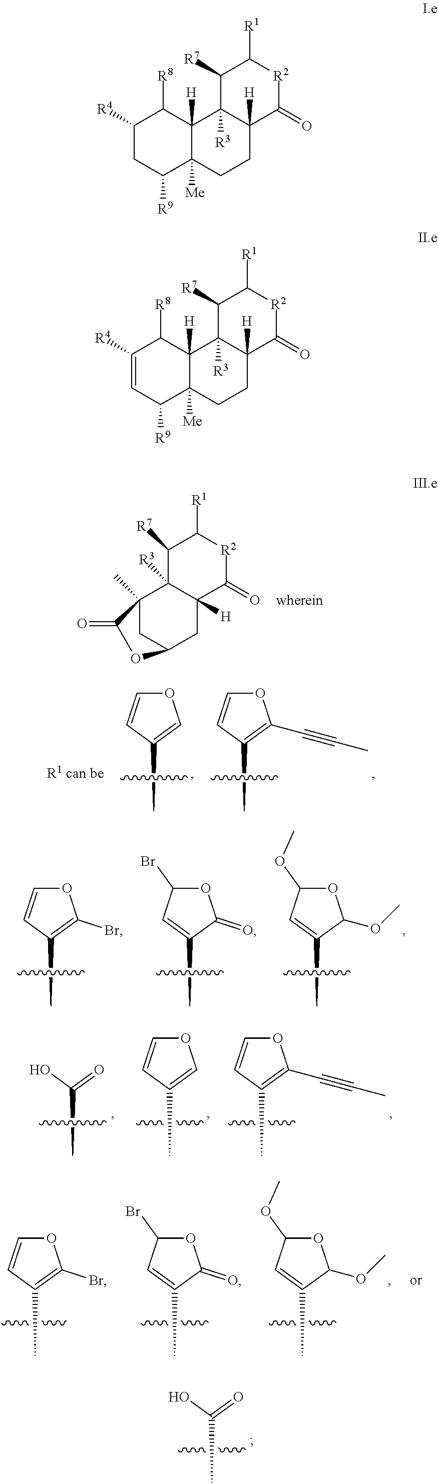

R² can be O or CH₂;
R³ can be H or CH₃;
R⁴ can be OH, OAc,

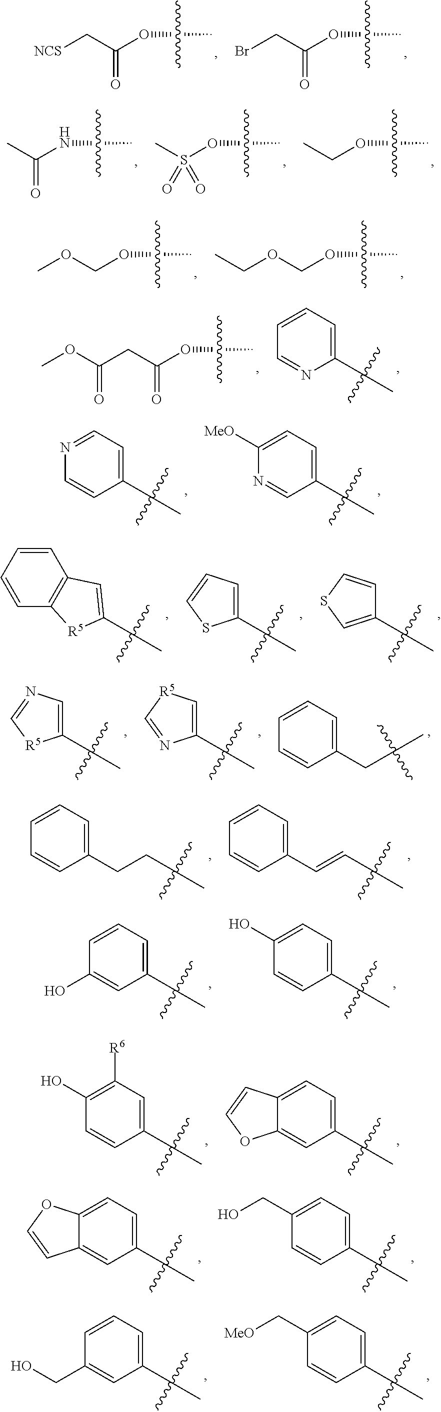

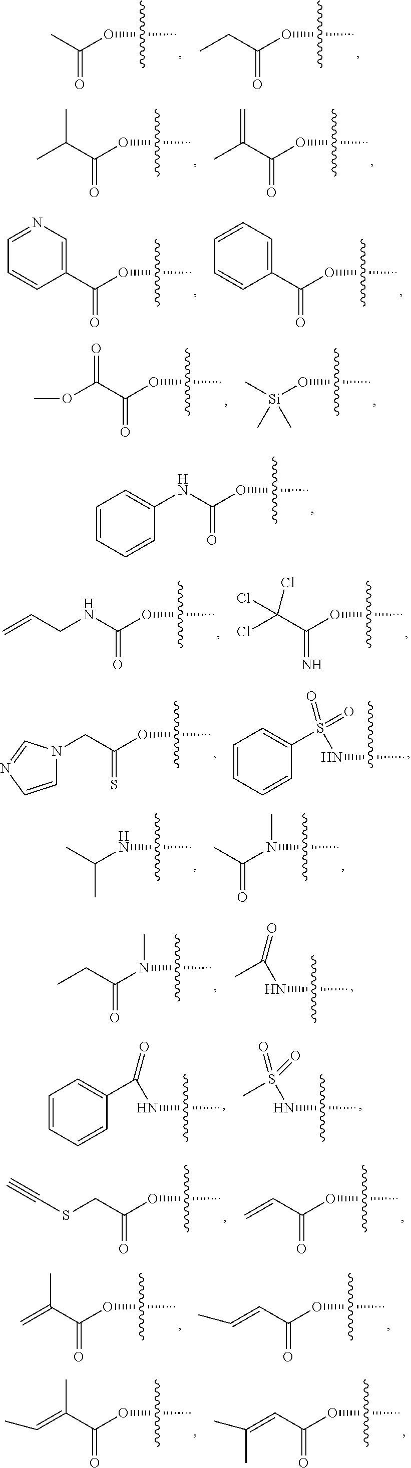

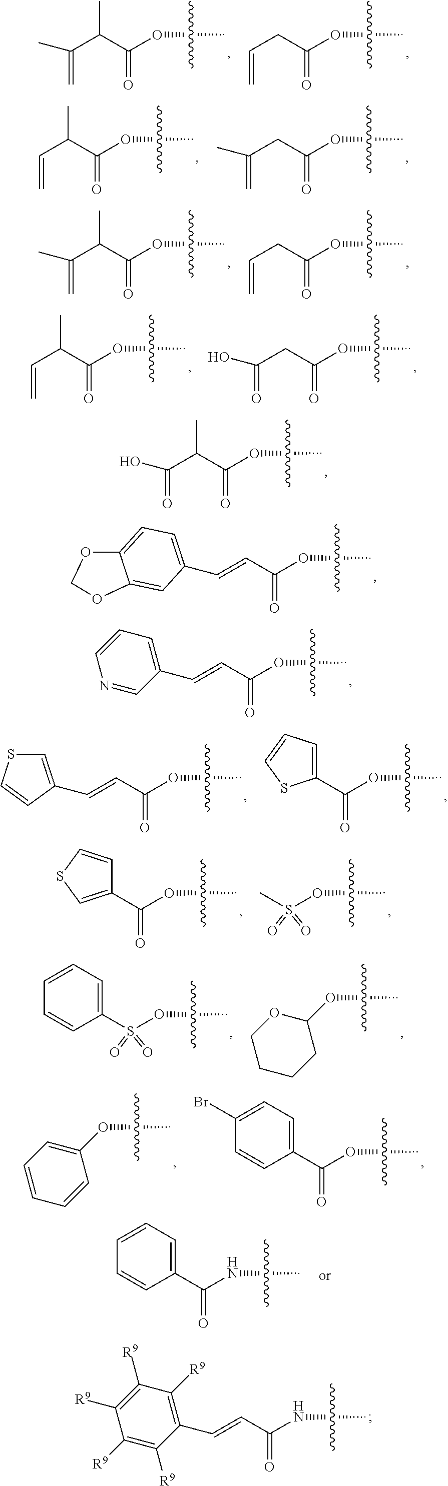

wherein R⁵ can be O or S; R⁶ can be F, Cl or Br; and each R⁹ can independently be H, OMe, NO₂, or CF₃;
R⁷ can be

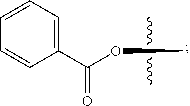

R⁸ can be

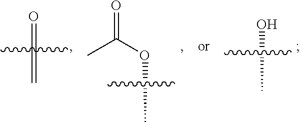

R⁹ can be CO₂Me or CONH₂; and
NCS is N-chlorosuccinimide; and OAc is

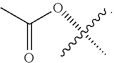

In some embodiments, the compound of Formula I, or a pharmaceutically acceptable salt thereof, can have the structure of Formula I.b.

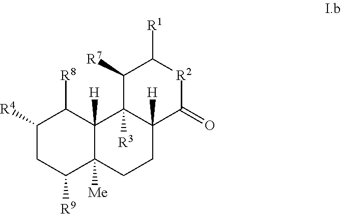

In some embodiments, the compound of Formula II, or a pharmaceutically acceptable salt thereof, can have the structure of Formula II.b.

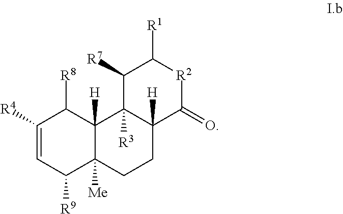

In some embodiments, the compound of Formula III, or a pharmaceutically acceptable salt thereof, can have the structure of Formula III.b.

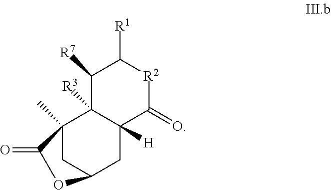

In some embodiments of Formula I.b, Formula II.b, or Formula III.b, or a pharmaceutically acceptable salt of any of the foregoing, the stereochemistry of R¹ can be one of the following:

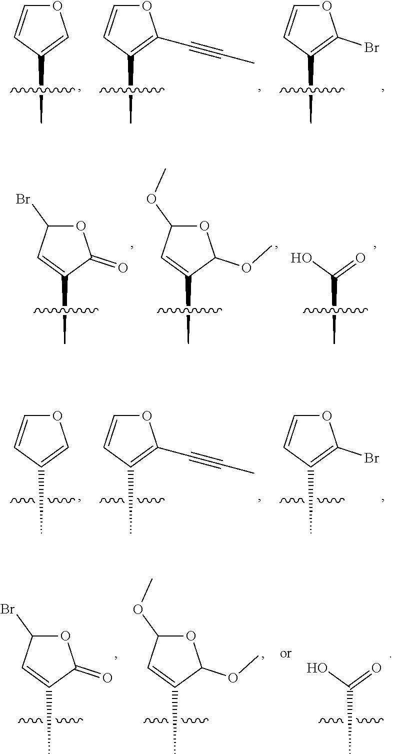

In some embodiments Formula I.b, Formula II.b, or Formula III.b, or a pharmaceutically acceptable salt of any of the foregoing, the stereochemistry of R⁴ can be one of the following:

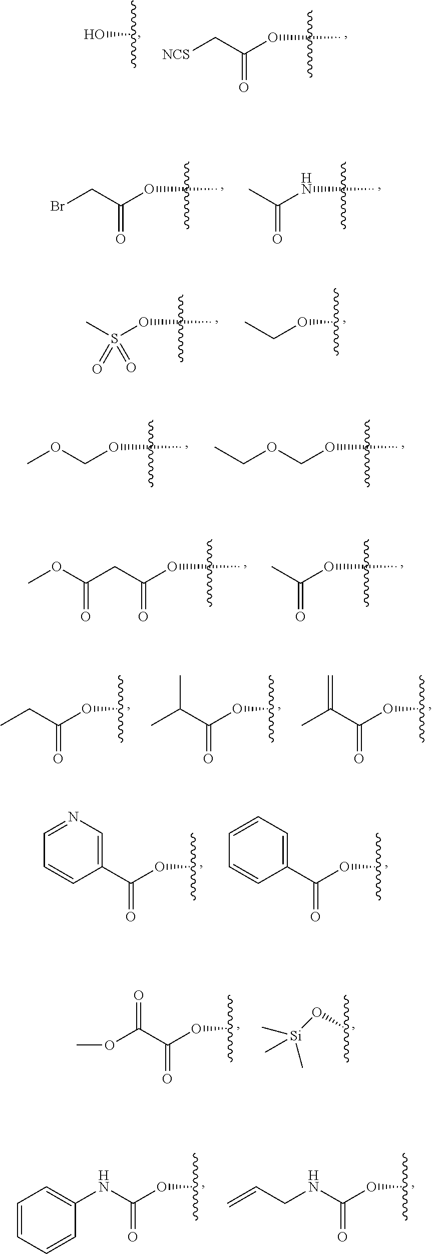

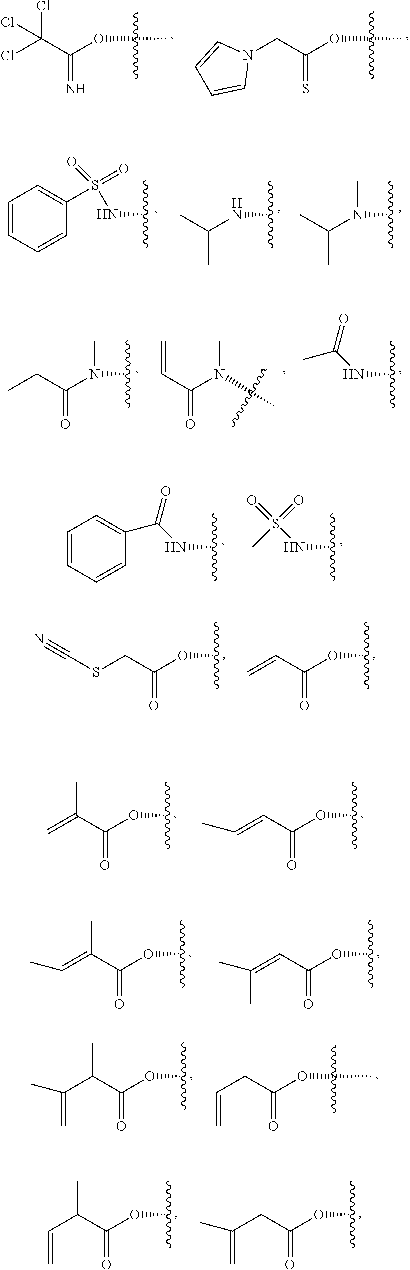

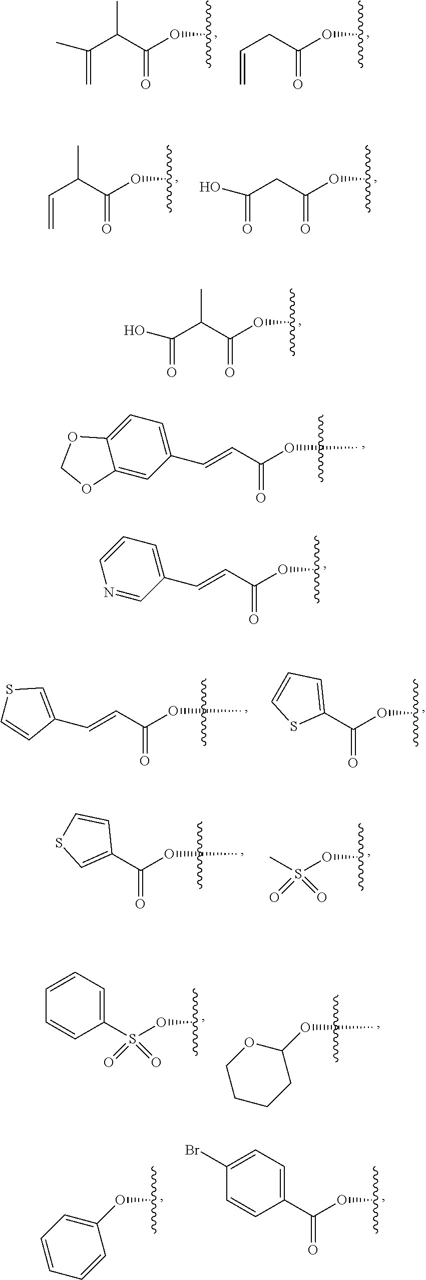

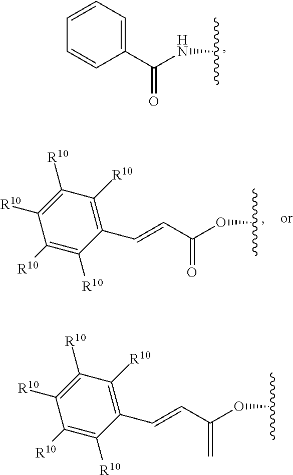

In some embodiments of Formula I.b, Formula II.b, or Formula III.b, or a pharmaceutically acceptable salt of any of the foregoing, the stereochemistry of R⁷ can be

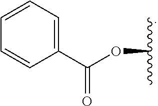

In some embodiments of Formula I.b, Formula II.b, or Formula III.b, or a pharmaceutically acceptable salt of any of the foregoing, the stereochemistry of R⁸ can be one of the following:

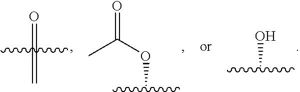

In some embodiments, the compound of Formula I.b, or a pharmaceutically acceptable salt thereof, can be a stereoisomer of O6C-20-nor-SalA selected from:

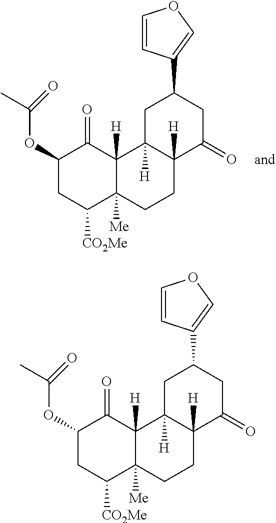

(including pharmaceutically acceptable salts of any of the foregoing).

In some embodiments, the compound of Formula I.b, or a pharmaceutically acceptable salt thereof, can be enantiomerically enriched

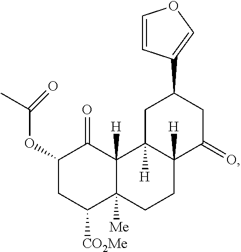

or a pharmaceutically acceptable salt thereof. In some embodiments, the compound of Formula I.b, or a pharmaceutically acceptable salt thereof, can be enantiomerically pure

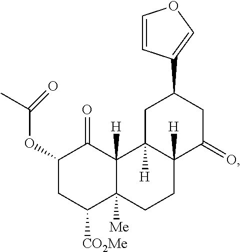

or a pharmaceutically acceptable salt thereof. As used herein, “enantiomerically enriched” means that one enantiomer is obtained in excess of the other enantiomer. In some embodiments, an enantiomerically enriched mixture has an enantiomeric excess of over 0%, preferably over 20%, preferably over 40%, preferably over 70%, more preferably over 80%, more preferably over 90% or more preferably over 95% of one enantiomer in relation to the other enantiomer. An “enantiomerically pure” compound is considered as a mixture of two enantiomers where said mixture is composed of over 95%, preferably over 98%, more preferably over 99%, or more preferably over 99.5% of one enantiomer.

In some embodiments, the compound of Formula I.b, Formula II.b, or Formula II.b, or a pharmaceutically acceptable salt of any of the foregoing, can be selected from:

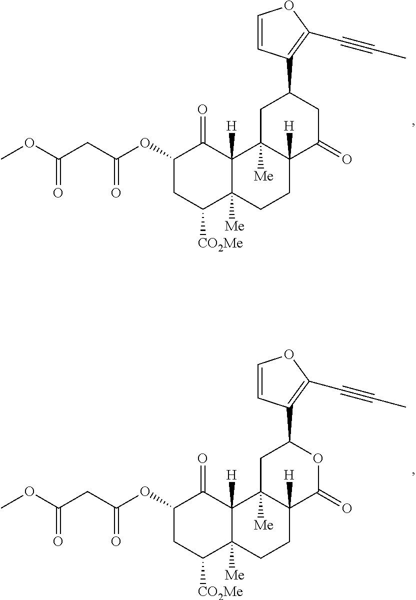

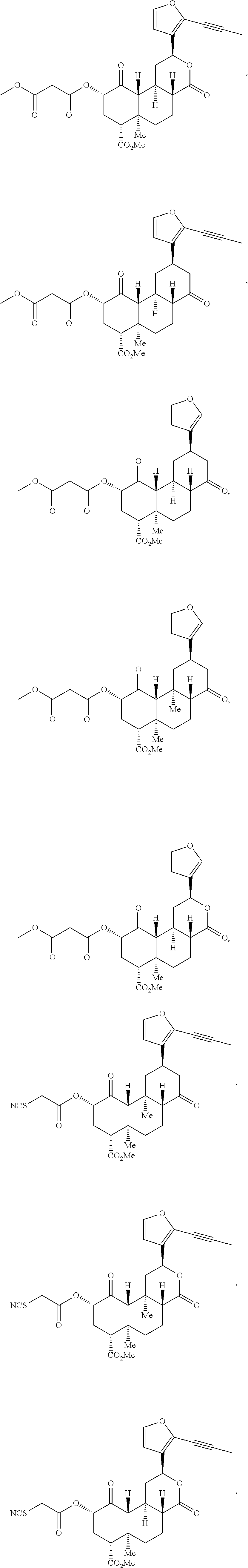

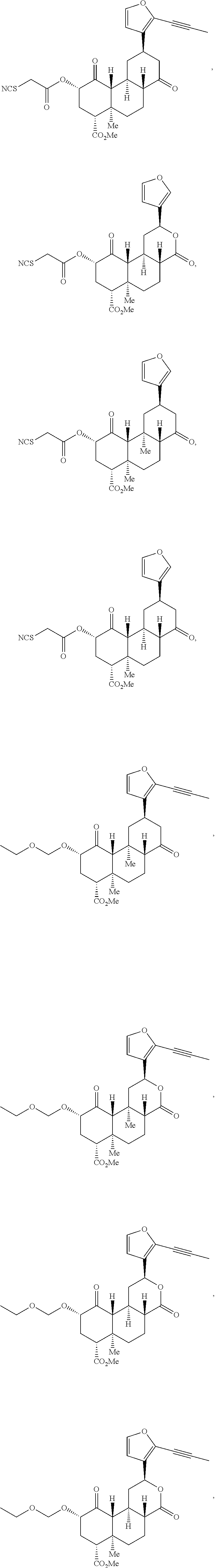

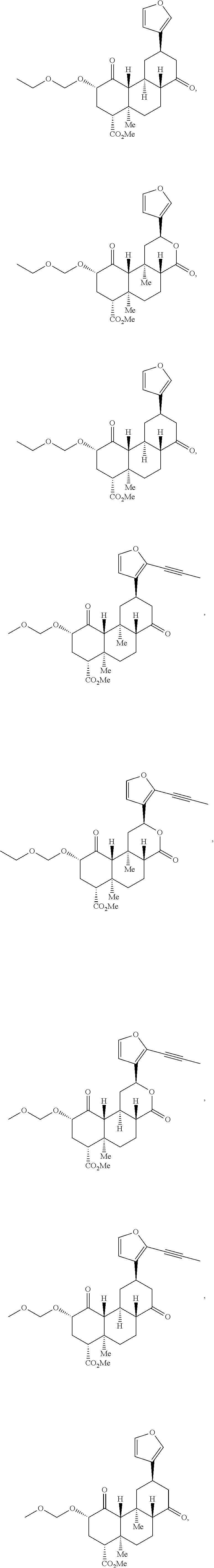

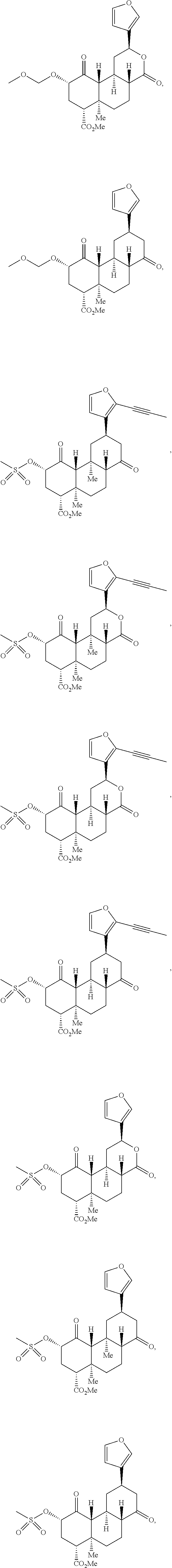

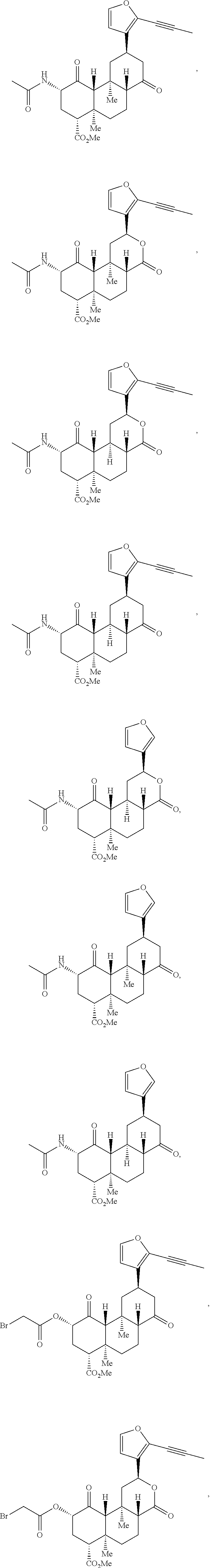

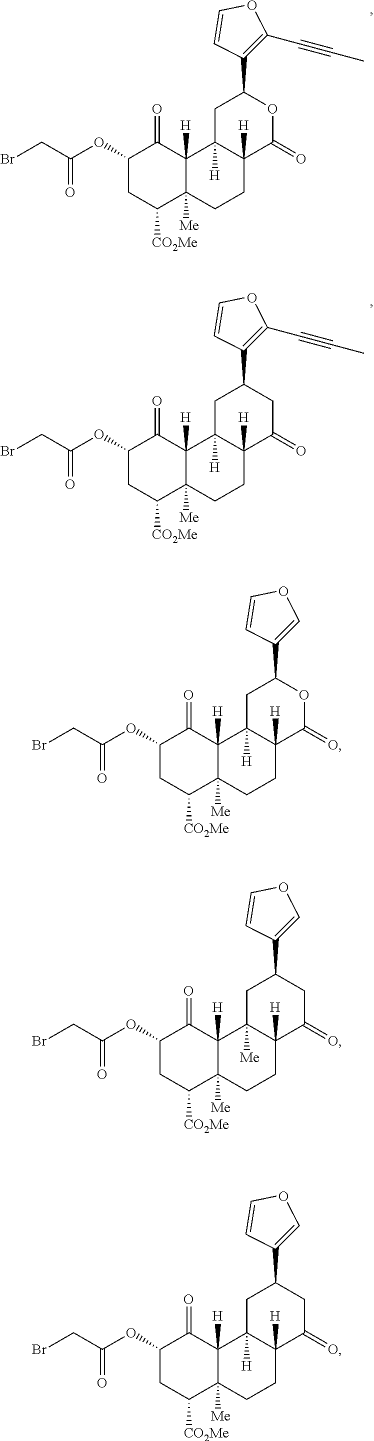

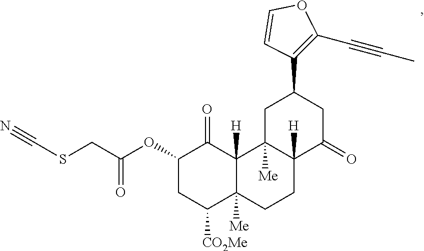

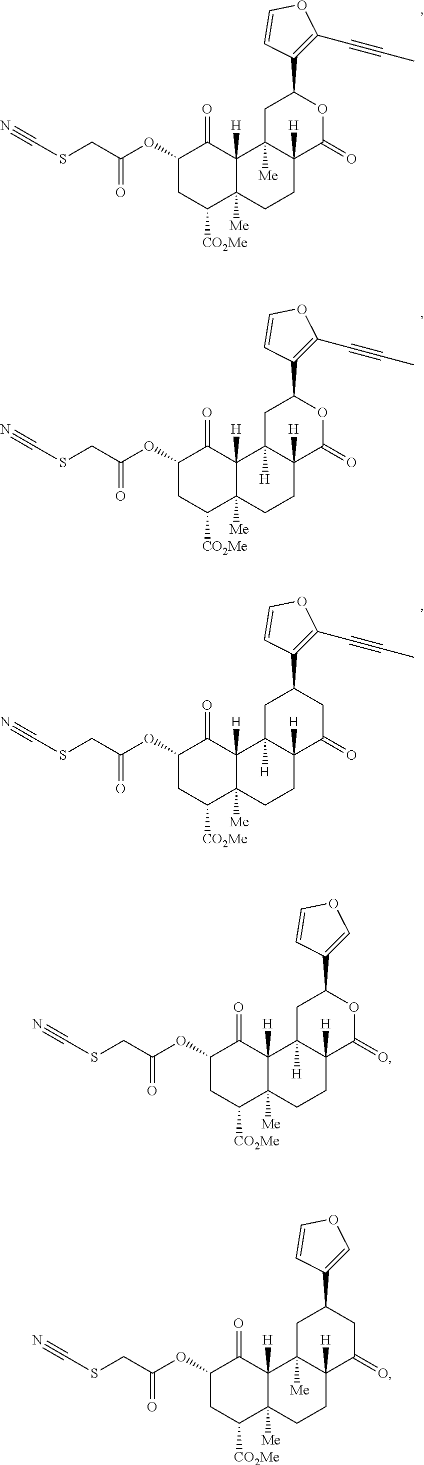

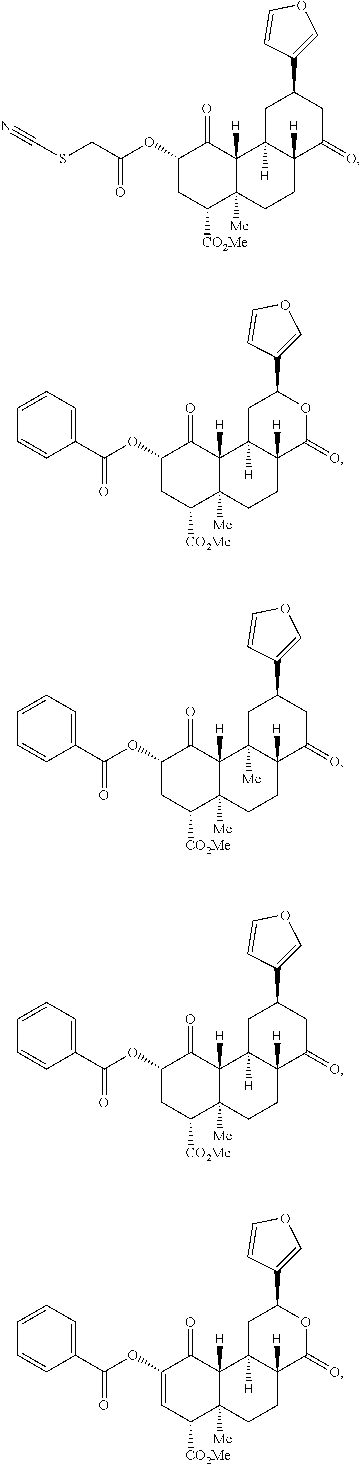

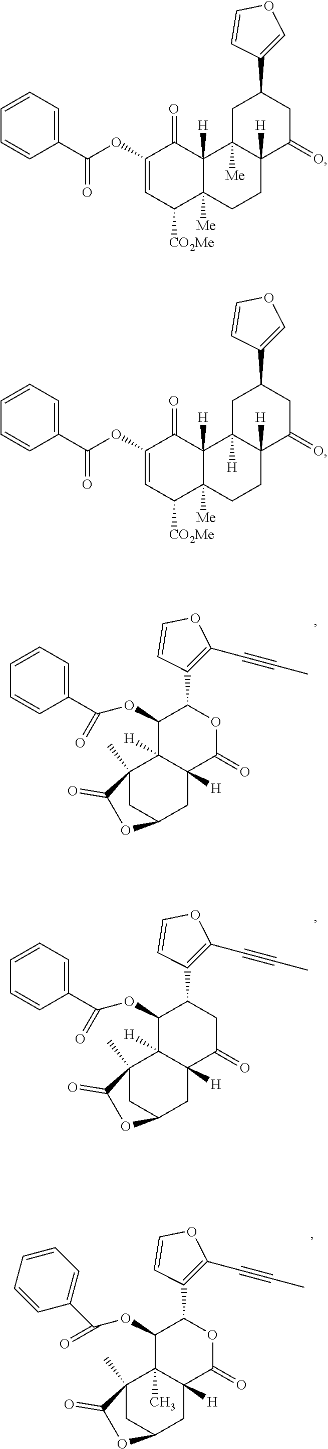

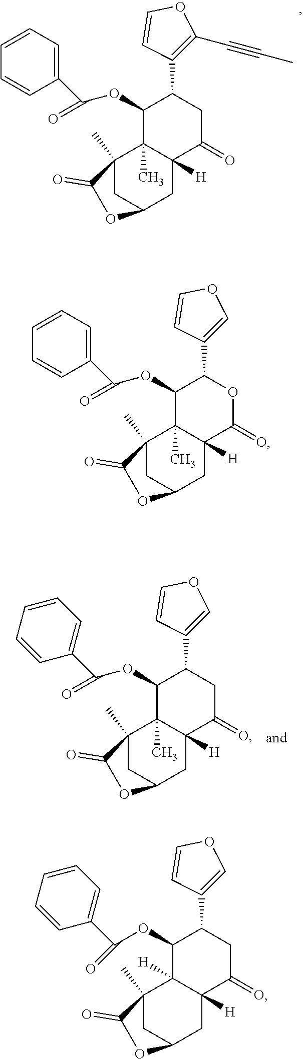 [SMILES: CC#Cc1occc1[C@@H]1CC(=O)[C@@H]2C[C@@H]3C[C@@](C)(C(=O)O3)[C@@]2(C)[C@H]1OC(=O)c1ccccc1 ; C[C@]12[C@@H](C[C@@H]3C[C@@]1(C)C(=O)O3)C(=O)O[C@@H](c1ccoc1)[C@@H]2OC(=O)c1ccccc1 ; C[C@]12[C@@H](C[C@@H]3C[C@@]1(C)C(=O)O3)C(=O)C[C@@H](c1ccoc1)[C@@H]2OC(=O)c1ccccc1 ; C[C@@]12C[C@@H](C[C@H]3C(=O)C[C@@H](c4ccoc4)[C@H](OC(=O)c4ccccc4)[C@@H]31)OC2=O]

a pharmaceutically acceptable salt of any of the foregoing.

In other embodiments of Formula I.b, Formula II.b, or Formula III.b, or a pharmaceutically acceptable salt of any of the foregoing, the compound can be selected from:

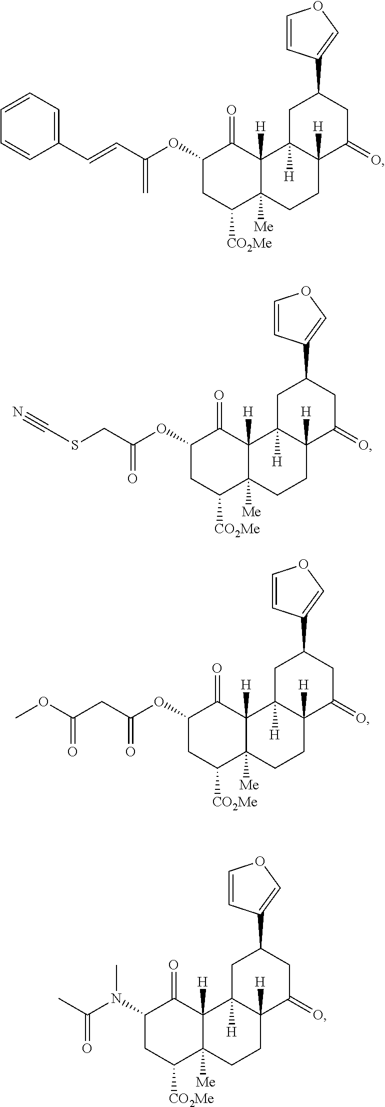

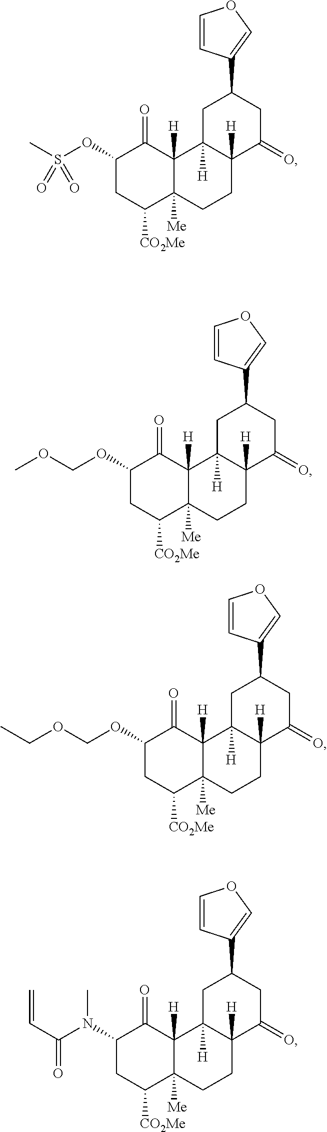

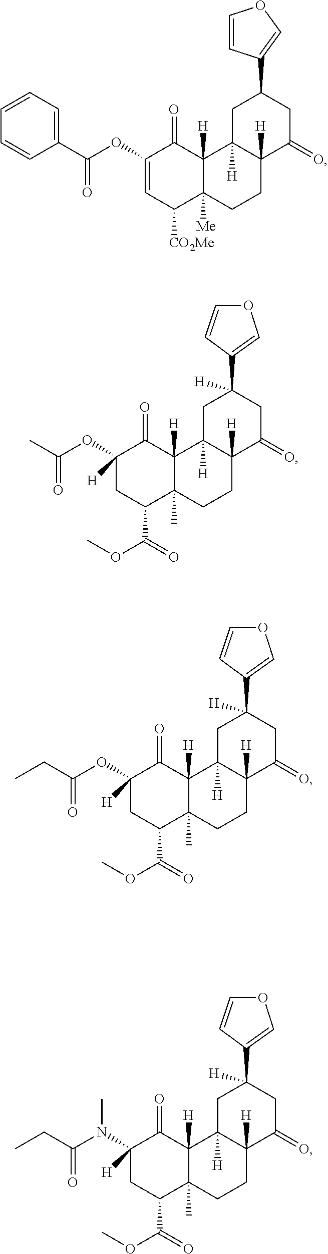

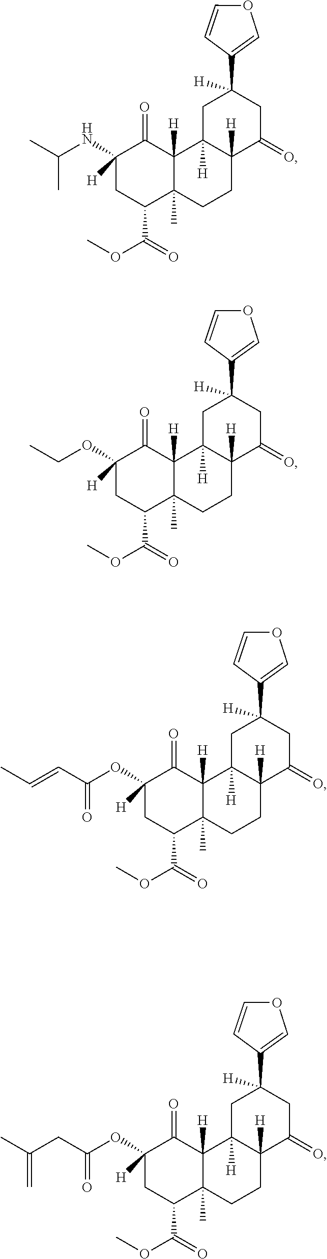

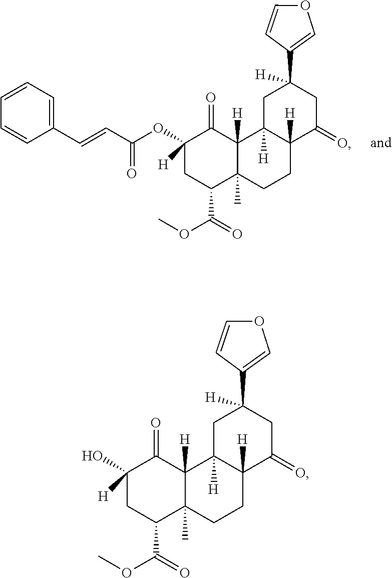

or a pharmaceutically acceptable salt of any of the foregoing.

There are several drawbacks associated with Salvinorin A. Exemplary drawbacks of SalA include poor stability (for example, likelihood of epimerization), short half-life, short brain resonance time, and hallucinations. Accordingly, there is a need for compounds such as those described herein that address one or more the drawbacks associated with Salvinorin A.

Some advantages of a compound described herein can include increased half-life (for example, by addition of one or more halogen atoms or lipophilic moieties), greater solubility (for example, by addition of one or more carbonyl and/or hydroxyl groups), higher affinity for KOR and/or MOR receptors, higher efficacy for KOR and/or MOR receptors, greater stability, and lower cytotoxicity. For example, these advantages can be relative to the naturally occurring Salvinorin A.

### Pharmaceutical Compositions

Some embodiments described herein relate to a pharmaceutical composition that can include an effective amount of one or more compounds described herein (such as 1, 2 or 3 compounds of Formula I, Formula II, and/or Formula III, or a pharmaceutically acceptable salt of any of the foregoing), and a pharmaceutically acceptable carrier, diluent, excipient and/or combination thereof. In some embodiments, the pharmaceutical composition can include at least one compound described herein, or a pharmaceutically acceptable salt thereof (such as 1, 2 or 3 compounds of Formula I, Formula II and/or Formula III, or pharmaceutically acceptable salts of any of the foregoing). In some embodiments, a pharmaceutical composition include at least two compounds of Formula I, or a pharmaceutically acceptable salt thereof (such as 2 or 3 compounds of Formula I, or pharmaceutically acceptable salts thereof). In some embodiments, a pharmaceutical composition include at least two compounds of Formula II, or a pharmaceutically acceptable salt thereof (such as 2 or 3 compounds of Formula II or pharmaceutically acceptable salts thereof). In other embodiments, a pharmaceutical composition include at least two compounds of Formula III, or a pharmaceutically acceptable salt thereof (such as 2 or 3 compounds of Formula III, or pharmaceutically acceptable salts thereof). In some embodiments, a pharmaceutical composition can include at least two compounds from Formula I, Formula II, or Formula III, or a pharmaceutically acceptable salt of any of the foregoing (such as 2 compounds of Formula I, 2 compounds or Formula II, or 1 compound or Formula I and 1 compound of Formula II, or pharmaceutically acceptable salts of any of the foregoing).

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic, or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine. N-methyl-D-glucamine, tris(hydroxymethyl)methylamine, C₁-C₇ alkylamine, cyclohexylamine, triethanolamine, ethylenediamine, and salts with amino acids such as arginine and lysine. For compounds of Formula I, those skilled in the art understand that when a salt is formed by protonation of a nitrogen-based group (for example, NH₂), the nitrogen-based group can be associated with a positive charge (for example, NH₂ can become NH₃) and the positive charge can be balanced by a negatively charged counterion (such as Cl⁻).

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability, etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

In some embodiments, the pharmaceutical compositions described herein can be administered to a subject per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral (enteral), transmucosal (nasal, vaginal, rectal or sublingual), pulmonary, topical, transdermal (through a patch), aerosol, injection, infusion and parenteral delivery, including intramuscular, subcutaneous, intravenous (IV), intramedullary injections, intrathecal, direct intraventricular, intraperitoneal, intranasal and intraocular injections. In some embodiments, a compound of Formula I, or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container and labeled for treatment of an indicated condition.

### Synthesis

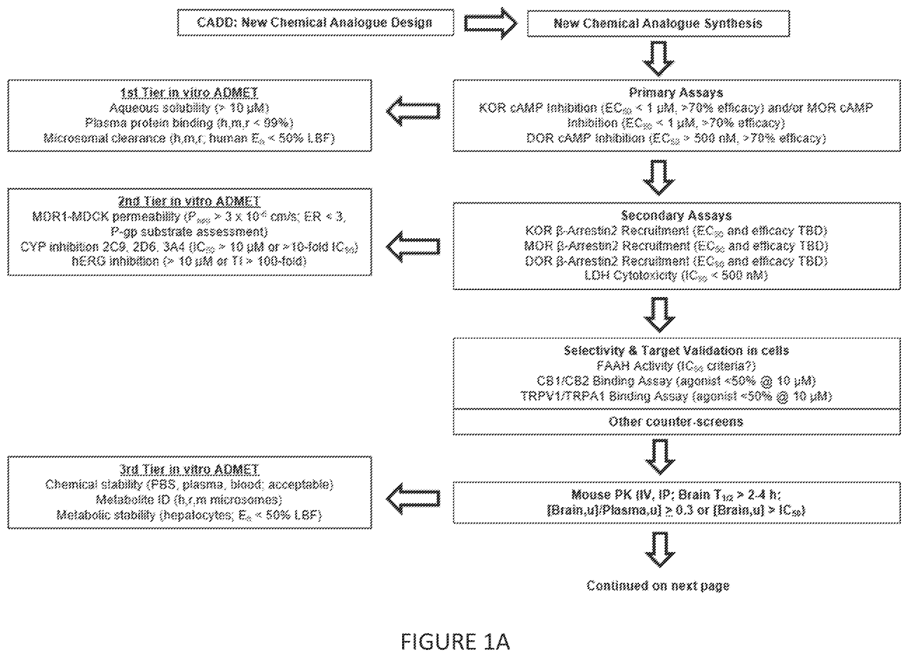

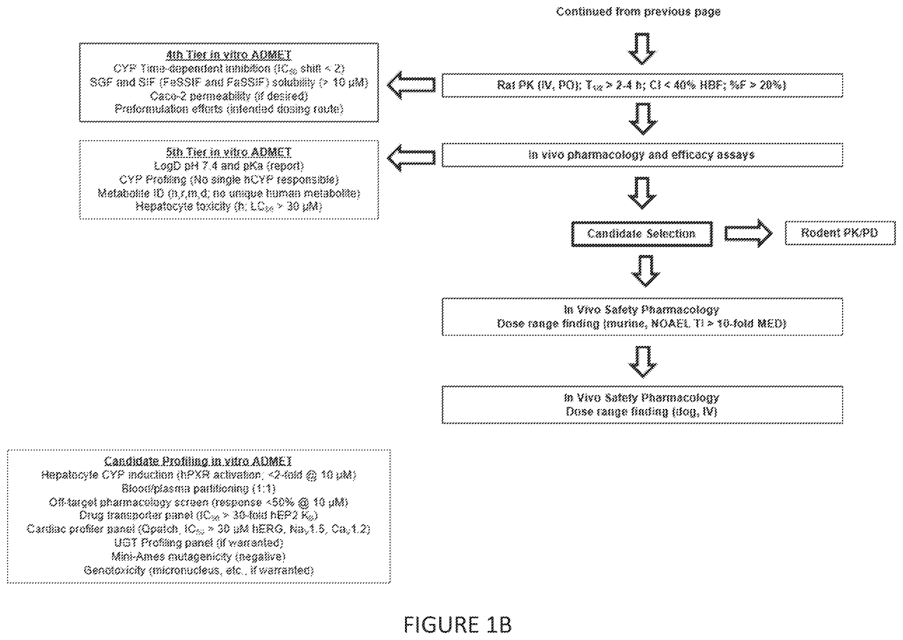

FIGS. 1A and 1B show an example of a flow chart for synthesizing and testing the various compounds provided herewith. In brief, the compounds are first synthesized, then tested for their physical and biochemical properties. After that, they are tested in cell culture then in animal models for efficacy.

In some embodiments, compounds described herein, along with pharmaceutically acceptable salts thereof, are synthesized by chemical methods. Such methods are known in the art. See, e.g., Crowley R S et al., ACS Chem. Neurosci DOI: 10.1021/acschemneuro.0c00191 (2020); Beguin C et al., Bioorganic & Medicinal Chemistry Letters 16:4679-4685 (2006); Riley A P et al., J. Med. Chem. 57:10464-10475 (2014); Hill Si et al., Nat. Prod. Rep. DOI: 10.1039/d0np00028k (2020). In other embodiments, the compounds are synthesized biosynthetically, e.g., in transgenic plants, yeast or bacteria by methods known in the art. See Jamieson C S et al., Chem. Soc. Rev. DOI: 10.1039/d1cs00065a (2021); Kutrzeba L. et al., Phytochemistry 68:1872-1881 (2007); and Pelot K A. et al., The Plant Journal 89:885-897 (2017).

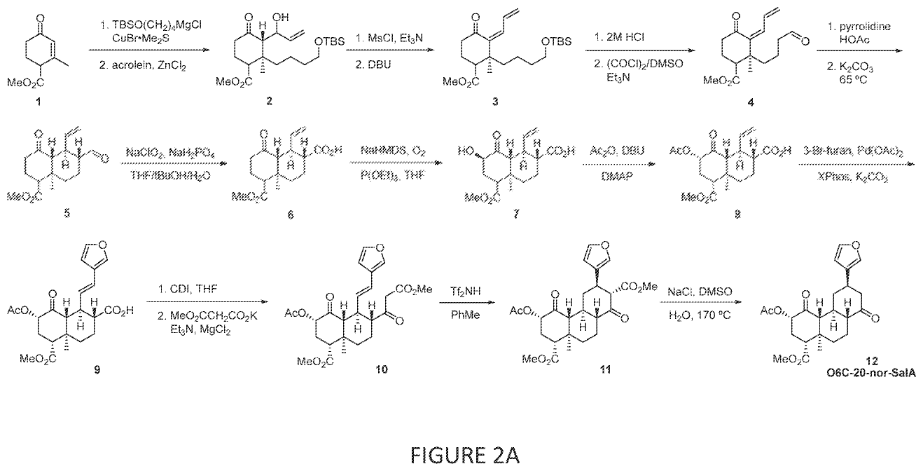

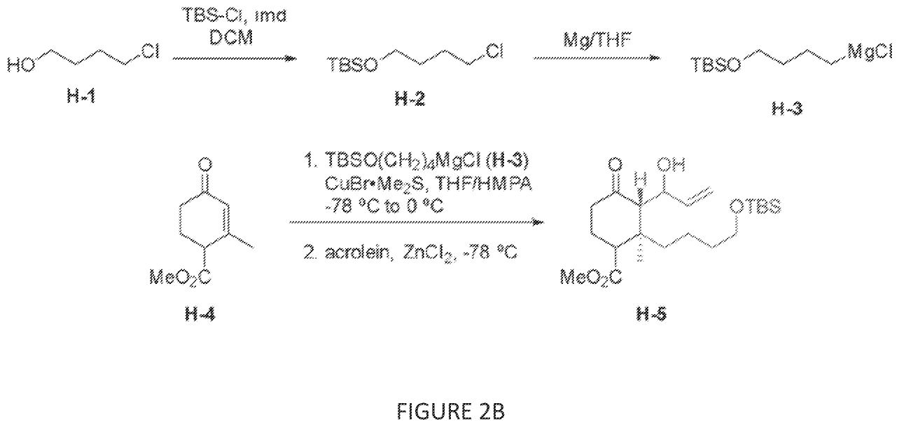

Non-limiting examples of chemical synthesis methods are depicted in FIGS. 2-11. Compounds of Formulae I, II and III (including pharmaceutically acceptable salts thereof) along with those described herein may be prepared in various ways. General synthetic routes for preparing Formulae I, II and III (including pharmaceutically acceptable salts thereof) are shown and described herein along with some examples of starting materials used to synthesize compounds described herein. Non-limiting examples of chemical synthesis are depicted in FIGS. 2-11. Additionally, for the purpose of the general synthetic routes, the structures depicted are appropriately protected, as known by one skilled in the art and the generic structures are meant to include these protecting groups. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

FIG. 2A depicts a synthetic route from starting compound Hagemann ester to racemic O6C-20-nor-SalA. In the following description of FIGS. 2A and 2B, compounds 1-12 refer to the compounds labeled as such in FIGS. 2A and 2B. Hagemann ester 1 can be subjected to a multi-component conjugate addition/alkylation reaction with cuprate formed from (4-((tert-butyldimethylsilyl)oxy)butyl)magnesium chloride followed by addition of acrolein to produce the allylic alcohol 2. Elimination of the allylic alcohol of 2 can be accomplished by treatment with methanesulfonyl chloride and an organic base to form the (R)-diene cyclohexane 3. Compound 3 can undergo deprotection of the silyl ether under acidic hydrolysis conditions to produce an intermediate primary alcohol, which can be subjected to oxidation conditions to produce the primary aldehyde 4. Cyclization of 4 under Knovenagel conditions followed by a thermal equilibration process can produce the bicyclic aldehyde 5 in which the ring junction stereochemistry can be set. Pinnick oxidation of 5 can provide the carboxylic acid bicycle 6, which can be subjected to oxidation conditions to provide α-ketoalcohol 7, in which the stereochemistry can be defined as (R) at the C2 alcohol carbon. Acetoxylation of the alcohol in the presence of an organic base under thermal conditions can produce α-acetoxy ketone 8 in which the stereochemistry at the C2 position can be inverted. Heck reaction of the terminal alkene of 8 with 3-bromofuran can provide furanyl adduct 9, which can be subsequently converted to the β-ketoester 10 with monomethyl malonate. Compound 10 can undergo a Lewis acid-catalyzed hydroalkylation cyclization reaction to provide tricyclic adduct 11, which can be subjected to decarboxylation conditions to produce compound 12.

FIG. 2B depicts a method for preparing the allylic alcohol 2 referred to in the description of FIG. 2A by Grignard reaction. Experimental data of the synthesis depicted in FIG. 2B is included in Table 1. In Table 1, compounds H-1 to H-5 refer to the compounds labeled as such in FIG. 2B.

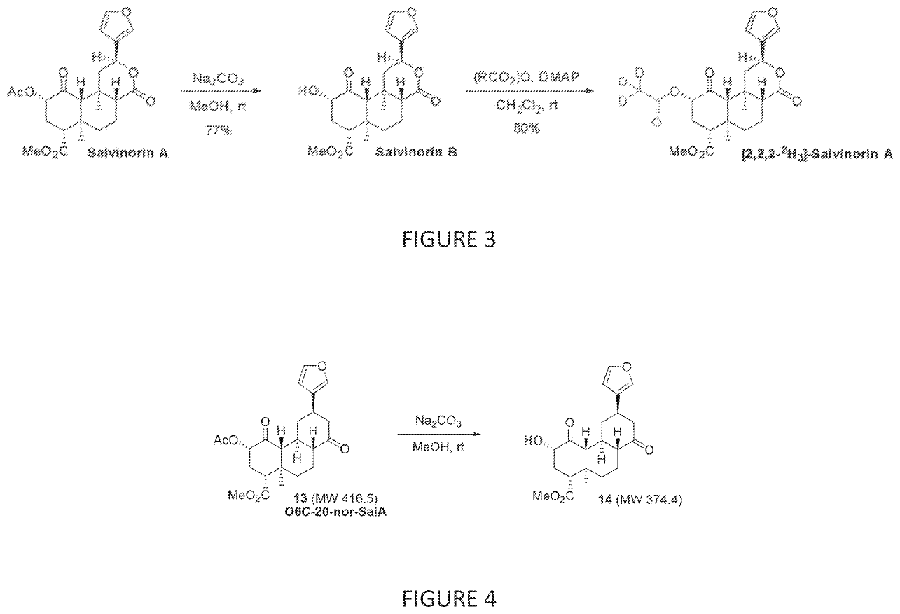

FIG. 3 depicts a method for preparing per-deuterated-acetyl Salvinorin A. The conditions described in FIG. 3 do not hydrolyze the methyl ester or lactone core. The method depicted in FIG. 3 can be adapted for any of the compounds described herein without undue experimentation.

FIG. 4 depicts a synthetic route from O6C-20-nor-SalA to O6C-20-nor-SalB.

In the following description, compounds 13 and 14 refer to the compounds labeled as such in FIG. 4. In a single step, O6C-20-nor-SalA (compound 13) can be deacetylated at room temperature to form the secondary alcohol O6C-20-nor-SalB (compound 14). Experimental data of the described synthesis is included in Table 1. Those skilled in the art would understand that compound 14 may be used as an intermediate to form various embodiments described herein.

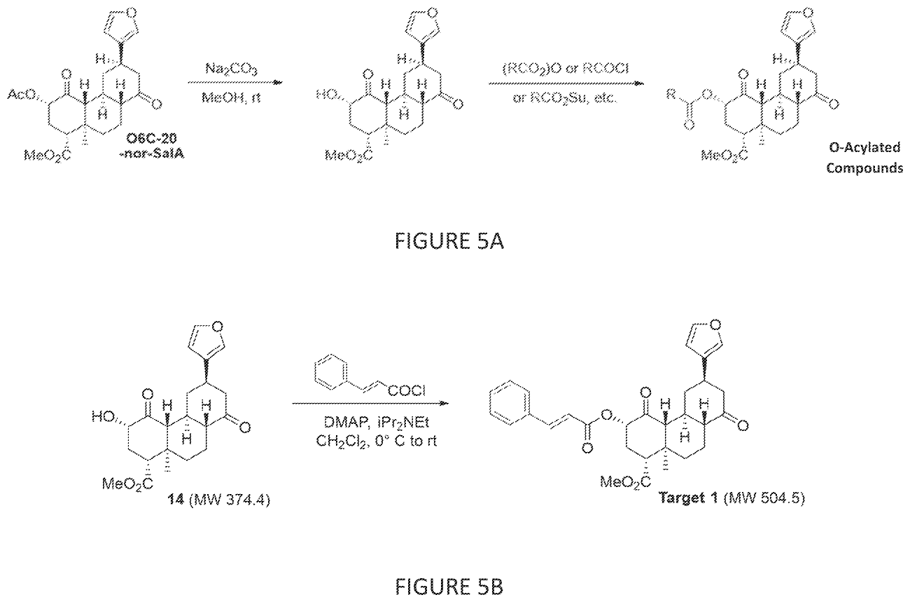

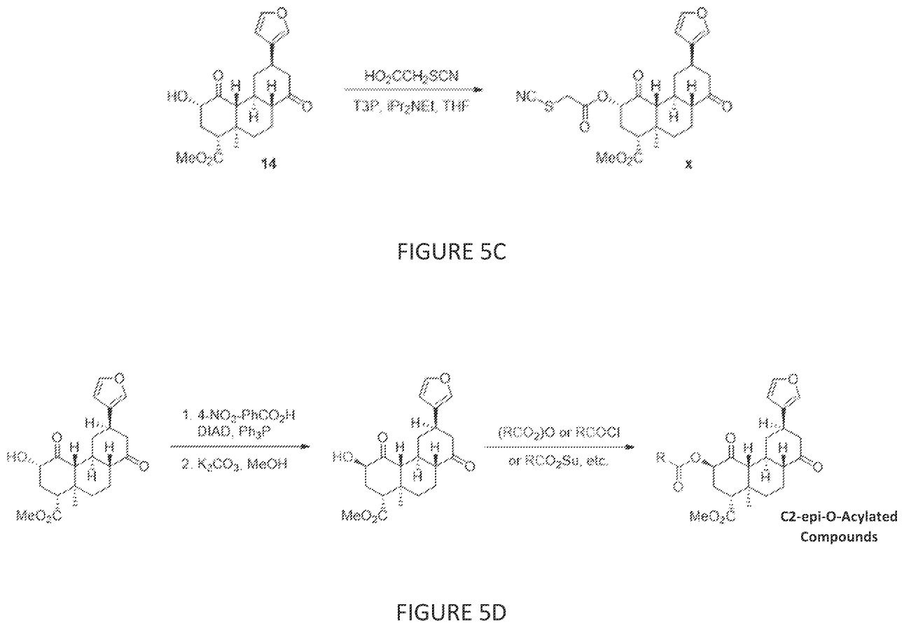

FIG. 5A depicts a synthetic route from O6C-20-nor-SalA to O-acylated derivatives at the C2 position. O6C-20-nor-SalA can be deacetylated at room temperature to form O6C-20-nor-SalB. Ester formation can be achieved using activated ester reagents, for example, acid chlorides and succinates and acyl anhydrides. Those skilled in the art would understand that various chemical methods may be used to form various embodiments described herein. See also, Bioorg. Med. Chem. Lett. 2004, 14, 5099; J. Med. Chem. 2005, 48, 4765; Bioorg. Med. Chem. Lett. 2015, 25, 4689; Tetrahedron Lett. 2010, 51, 5207.

FIG. 5B shows an exemplary synthesis from O6C-20-nor-SalB to an O-acylated compound referred to herein as Target 1. The hydroxyl group of O6C-20-nor-Sala (compound 14) can be acylated with cinnamoyl chloride in the presence of an organic base to form the O-acylated compound shown. Those skilled in the art would understand that various chemical methods may be used to form various embodiments described herein. Experimental data of the described synthesis is included in Table 1.

FIG. 5C shows an exemplary synthesis from O6C-20-nor-SalB to an O-acylated compound referred to herein as Target X. The hydroxyl group of O6C-20-nor-SalB (compound 14) can be acylated with 2-thiocyanatoacetic acid in the presence of an acid activating agent and an organic base to form the O-acylated compound shown.

FIG. 5D shows a proposed synthesis from O6C-20-nor-SalB to a C2-epi-O-acylated compound referred to herein as Target Y. The hydroxyl group of O6C-20-nor-SalB can be epimerized and acylated to form C2-epi-O-acylated compounds.

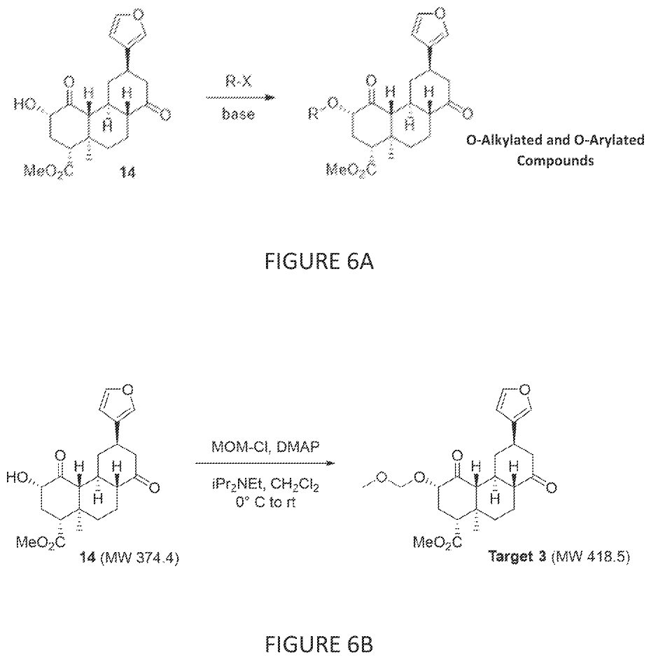

FIG. 6A shows a proposed synthesis from O6C-20-nor-SalB to various embodiments described herein. The hydroxyl group of O6C-20-nor-SalB (compound 14) can be alkylated to form O-alkylated compounds. Alkyl ether formation can be achieved using activated alkane reagents, for example, alkyl halides and alkyl sulfonates, or by using O-alkylation chemical methods that those skilled in the art would understand to form various embodiments described herein. Alkyl ether formation can be achieved using activated aryl reagents by using O-arylation chemical methods known to those skilled in the art to form various embodiments described herein.

FIG. 6B shows an exemplary synthesis from O6C-20-nor-SalB to an O-alkylated compound referred to herein as Target 3. The hydroxyl group of O6C-20-nor-SalB (compound 14) can be alkylated with methoxymethyl chloride to form the O-alkylated compound shown. Those skilled in the art would understand that various chemical methods may be used to form various embodiments described herein. Experimental data of the described synthesis is included in Table 1. See also, Bioorg. Med. Chem. Lett. 2005, 15, 3744; Bioorg. Med. Chem. Lett. 2012, 23, 1023; Bioorg. Med. Chem. 2008, 16, 1279.

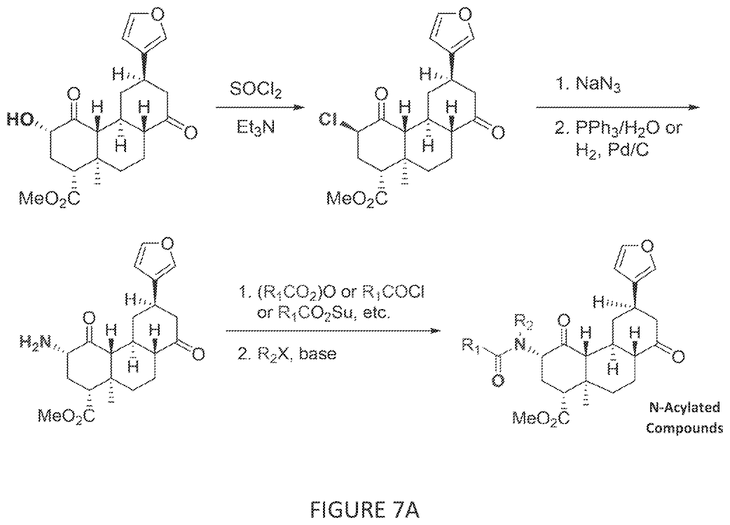

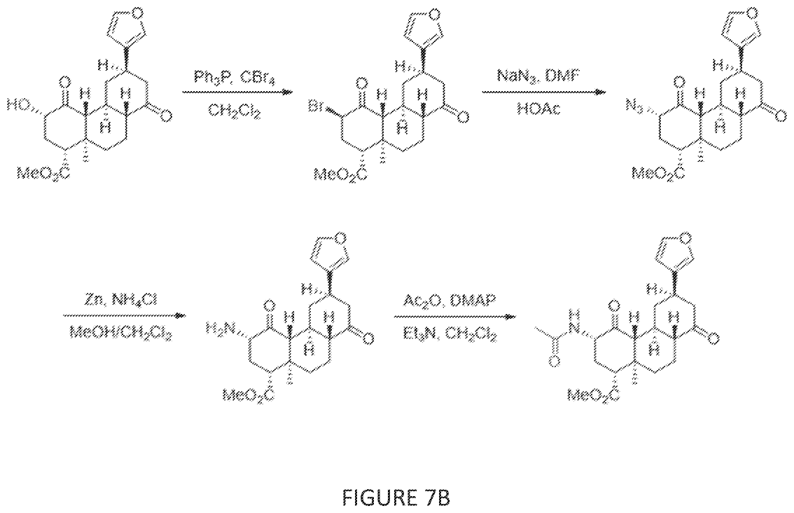

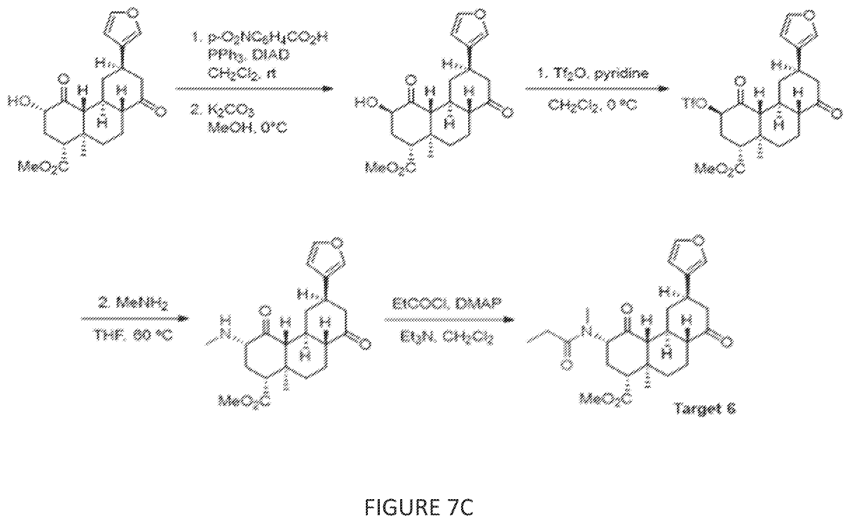

FIG. 7A depicts a proposed method for N-acylation of O6C-20-nor-SalB to various embodiments described herein. This method takes advantage of the double-inversion of the stereochemistry at C2. The hydroxyl group of O6C-20-nor-SalB can be replaced with a halide with inversion of stereochemistry. The activated leaving group of the compound produced by the previous reaction can be displaced by a primary amine surrogate, again with inversion of stereochemistry. One method may displace the activated leaving group with an azide reagent followed by reduction to produce the primary amine shown. The amino group of the compound can be acylated to form N-acylated compounds, which can be understood to be reaction intermediates as well as various embodiments described herein. The amide compounds can be N-alkylated to form N-acyl-N-alkyl compounds. Those skilled in the art would understand that various chemical methods may be used at each step to form various embodiments described herein, and that various changes could be made in the above methods and compositions without departing from the scope of the present application. See also, Sharma et al., Bioorg. Med. Chem. 18:6886 (2010); Majer et al., Bioorg. Med. Chem. 22:256 (2014).

FIG. 7B shows an exemplary synthesis from O6C-20-nor-SalB to an N-acylated compound referred to herein as Target Z. The hydroxyl group of O6C-20-nor-SalB can be replaced with a halide with inversion of stereochemistry. The activated leaving group of the compound produced by the previous reaction can be displaced by a primary amine surrogate, again with inversion of stereochemistry, to produce a nitrogen-bound adduct with overall retention of configuration from O6C-20-nor-SalB. The activated leaving group can be reacted with an azide reagent followed by reduction to produce the primary amine shown in FIG. 7B. The primary amine group can be acylated to form N-acylated compounds, which can be understood to be reaction intermediates as well as various embodiments described herein. Those skilled in the art would understand that various chemical methods may be used at each step to form various embodiments described herein, and that various changes could be made in the above methods and compositions without departing from the scope of the present application. See also, J. Med. Chem. 2008, 51, 2421.

FIG. 7C shows an exemplary synthesis from O6C-20-nor-SalB to the compound referred to herein as Target 6. The secondary alcohol group of O6C-20-nor-SalB can undergo displacement with a carboxylic acid in the presence of a disubstituted azodicarboxylate or similar reagent and a trisubstituted phosphine, or with similar reagent combinations. This displacement can create an ester with inversion of configuration, and the adduct can subsequently be hydrolyzed to provide the secondary alcohol in which the alcohol stereochemistry has been inverted. The alcohol can be activated by appending functionality to make it a leaving group, for instance, an alkyl or aryl sulfonate, followed by addition of a nitrogen nucleophile to produce a secondary amine product in which the stereochemistry has again been inverted, with retention of configuration for the process initiated with O6C-20-nor-SalB. In the two-step process, the intermediate sulfonate may be isolated, or used in situ for a second stage amination. The amino group of the compounds produced by this step can be understood to be reaction intermediates as well as various embodiments described herein and can be alkylamines as well as arylamines. The amine moiety can be acylated to form N-acyl-N-substituted compounds. Those skilled in the art would understand that various chemical methods may be used at each step to form various embodiments described herein, and that various changes could be made in the above methods and compositions without departing from the scope of the invention. See also, Bioorg. Med. Chem. Lett. 2006, 16, 4679.

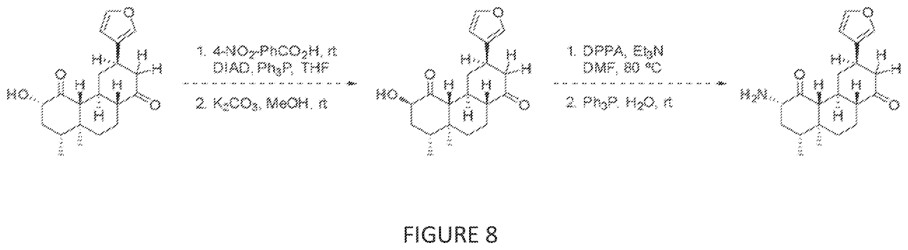

FIG. 8 depicts an additional proposed method for N-acylation of O6C-20-nor-salvinorin B to various embodiments described herein. The original Mitsunobu inversion/hydrolysis procedure with 4-nitrobenzoic acid can produce a Salvinorin B epimer, which can undergo a second inversion through a Mitsunobu amination with DPPA/Staudinger azide reduction two-step protocol to prepare C2-amino-Salvinorin A. These methods can be adapted for any of the compounds described herein without undue experimentation.

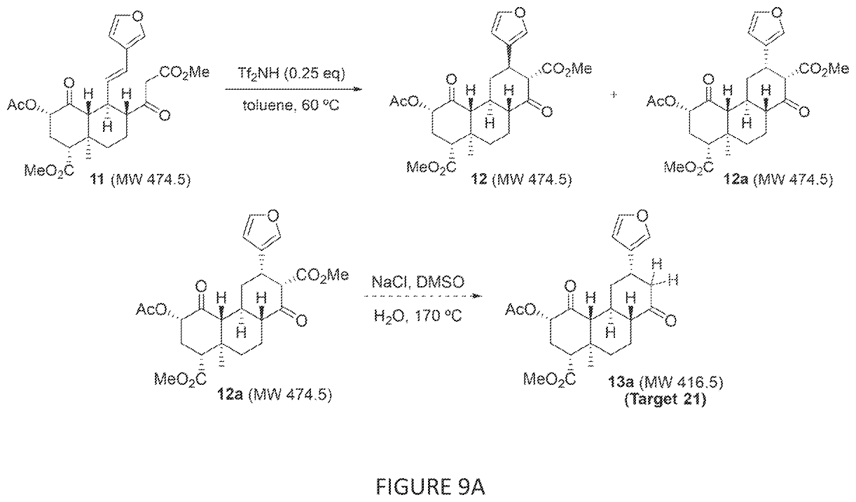

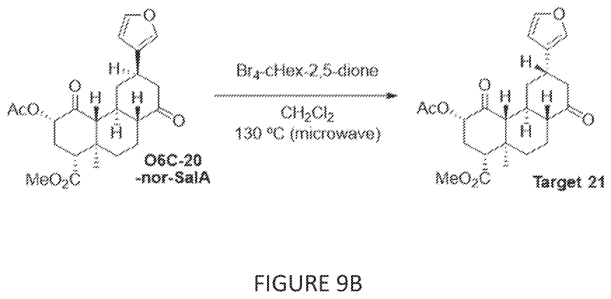

FIG. 9A shows a synthesis of the compound referred to herein as Target 21. In the first line of FIG. 9A, the penultimate step of the synthesis of racemic O6C-20-nor-SalA is shown. The epimer identified above can be isolated and then subjected to decarboxylation conditions to produce Target 21. Experimental data of the final step of the synthesis of Target 21 as shown in the second line of FIG. 9A is included in Table 1.

FIG. 9B shows an alternative synthesis of the compound referred to herein as Target 21. In the reaction, O6C-20-nor-SalA can be subjected to thermal conditions to epimerize the C12 carbon to form Target 21. See also, Bioorg. Med. Chem. 2012, 20, 31004.

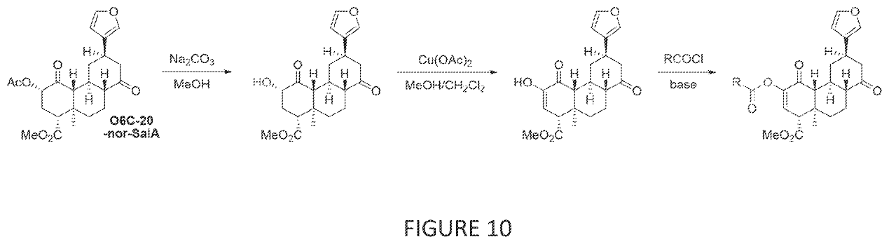

FIG. 10 shows a proposed synthesis from O6C-20-nor-SalA to various embodiments described herein. O6C-20-nor-SalA can be deacetylated to form O6C-20-nor-SalB. O6C-20-nor-SalB can be oxidized to the corresponding ketone, which exists largely in the enol tautomer. In the example shown, copper(II)acetate can be used for the oxidation process. The hydroxide group of the compound produced by the previous reaction can be selectively acylated to form various embodiments of multicyclic compounds described herein. See also, J. Med. Chem. Lett. 2016, 59, 11027; ACS Chem. Neurosci. 2020, 11, 1781.

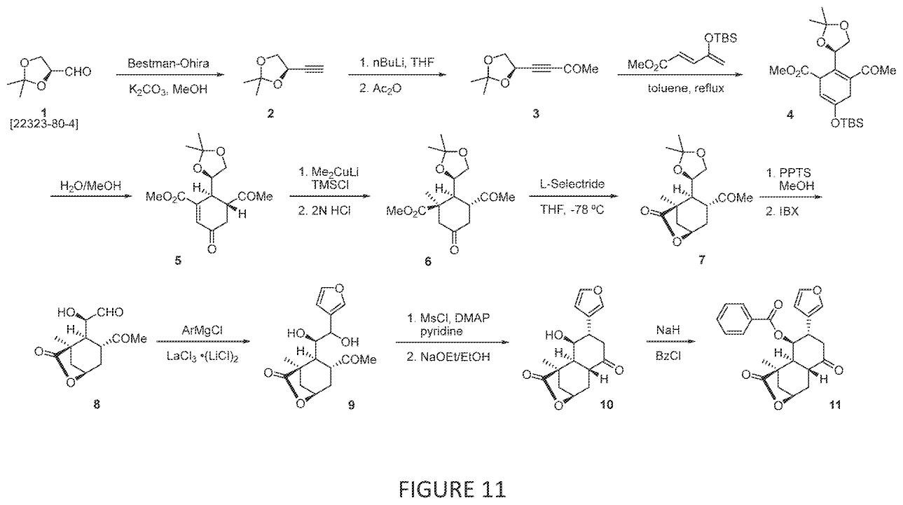

FIG. 11 shows a proposed synthesis from starting compound (S)-glyceraldehyde acetonide to the shown multicyclic compound. In the following description, compounds 1-11 refer to the compounds labeled as such in FIG. 11. A dialkyl (1-diazo-2-oxopropyl)phosphonate reagent can react with the aldehyde (compound 1) under basic Bestmann-Ohira conditions to produce the homologated alkyne compound 2. Homologated alkyne compound 2 can be acylated on the terminal alkyne carbon with an acyl halide or acyl anhydride and a strong base to produce propargyl ketone compound 3. Propargyl ketone compound 3 can be subjected to thermal Diels-Alder conditions with methyl (E)-4-(trisubstitutedsilyl)oxypenta-2,4-dienoate to produce the cyclohexadiene silyl ether compound 4, which can undergo hydrolysis of the silyl ether to produce the cyclohexene adduct compound 5. Cyclohexene adduct compound 5 can undergo selective alkylation with a cuprate reagent, in this case lithium dimethylcuprate, to provide cyclohexane compound 6 bearing a tetrasubstituted carbon at C3. Selective reduction of the ketone functionality, as with the aluminum hydride reagent shown, can produce an intermediate secondary alcohol alkoxide, which can undergo intramolecular acylation to produce bridged bicyclic product compound 7. Hydrolysis of the acetonide moiety followed by selective reduction of the primary can produce α-hydroxyaldehyde compound 8. The aldehyde moiety of α-hydroxyaldehyde compound 8 can be reacted with a Grignard reagent formed from 3-halofuran, preferably in the presence of a Lewis acid, to produce diol compound 9. The diol compound 9 can be converted to the cyclohexanone of compound 10 by a two-step process in which the secondary benzylic-like alcohol can selectively undergo activation by treatment with a sulfonyl halide reagent, after which the corresponding sulfonate can be displaced by the enolate of the methyl ketone in an intramolecular ring-closing under basic conditions to produce ketone of compound 10. The ketone of compound 10 can be acylated with an activated benzoic acid equivalent under basic conditions to form an embodiment of the multicyclic compound described herein. Those skilled in the art would understand that various changes could be made in the above methods and compositions without departing from the scope of the invention.

### Uses and Methods of Treatment

Opioid receptors including KOR and MOR are G protein-coupled receptors that are widely expressed throughout the central nervous system and brain, where they modulate a range of physiological processes including pain, inflammation, remyelination, stress response, and mood. See Dalefield et al., “The Kappa Opioid Receptor: A Promising Therapeutic Target for Multiple Pathologies” Frontiers in Pharm (2022) 13:Article 837671. Ligands in the endogenous opioid system, such as 3-endorphin, enkephalins, and dynorphins, bind KOR and MOR and modulate multiple biological responses in humans. KOR activation by agonists is coupled to the G protein Gᵢ/G₀, which increases phosphodiesterase activity. Phosphodiesterases can break down cAMP and produce an inhibitory effect in neurons. KORs can couple to inward-rectifier potassium or N-type calcium ion channels. Some KOR agonists have been shown to be MOR agonists.

Both KOR and MOR have been implicated in the progression and prevention of disease including, but not limited to, peripheral inflammation, neuroinflammation, blood clotting disorders, reduced blood flow, and substance use disorders. Classic opioid analgesics, such as morphine, act as a MOR agonist and are commonly used to treat moderate to severe acute pain associated with inflammation and an array of cancers. KOR agonists are of interest due to their non-addictive and anti-nociceptive properties, while dual KOR/MOR agonists, such as Eluxadoline, has been approved for the treatment of abdominal pain caused by irritable bowel syndrome. Some dual KOR and MOR agonists have been shown to have reduced side effects compared to Sal A. See Cichon et al., “Therapeutic Potential of Salvinorin A and Its Analogues in Various Neurological Disorders” Transl Perioper Pain Med (2022) 9(2):452-457. Inflammation is a common contributing factor to a host of human diseases including arthritis, asthma atherosclerosis, cancer, neurodegenerative diseases, stroke, and traumatic brain injury with peripheral immune cell infiltration. Targeting KOR and MOR with non-addictive and anti-nociceptive small molecules is a viable approach that can mitigate pain and inflammation associated with a disease and can improve the quality of life for those who suffer from these conditions.

KOR agonists have been shown to treat pain, myocardial infarction, pruritus, inflammation, edema, neuroinflammation (including HIV-induced), emesis, stroke and other brain injuries, hypoxic pulmonary hypertension, multiple sclerosis, substance use disorders (addiction), and osteoarthritis. See Beck et al., “Therapeutic Potential of Kappa Opioid Agonists” Pharmaceuticals (Basel) (2019)12(2):95. Additional indications can include treatment of mood conditions (such as stress, anxiety or depression), Alzheimer's disease, cognitive dysfunction, Parkinson's Disease, Tourette's Syndrome, immune-mediated diseases (such as arthritis, inflammation, diseases associated with the overproduction of cytokines such as IL-6, IL-1 and TNF-a), atopic dermatitis, GI disorders (such as inflammatory bowel disease (Crohn's, Ulcerative Colitis) and irritable bowel syndrome (IBS)), cancers (such as those involving the expression of vascular EGFR-2), hypoxia, ischemia, and cardiac dysfunction. See Dalefield et al., “The Kappa Opioid Receptor: A Promising Therapeutic Target for Multiple Pathologies” Frontiers in Pharm (2022) 13:Article 837671. KOR agonists can have been shown to treat acute kidney injury, such as renal ischemia-reperfusion injury. See Liu et al., “Kappa-opiod receptor agonist U50448H protects against renal ischemia-reperfusion injury in rats via activating the PI3K/Akt signaling pathway” Act Pharmacologica Sinica (2018) 39:97-106. KOR agonists can have been shown to treat cerebral artery dysfunction and constriction (such as pulmonary hypertension or cerebral vasospasm) via activation of nitric oxide synthase adenosine triphosphate-sensitive potassium channel. See Su et al., “Salvinorin A Produces Cerebrovasodilation through Activation of Nitric Oxide Synthase, k Receptor, and Adenosine Triphosphate-sensitive Potassium Channel” Anesthesiology (2011) 114(2):374-479 and Su et al., “Salvinorin A pretreatment preserves cerebrovascular autoregulation after brain hypoxic/ischemic injury via extracellular signal-regulated kinase/mitogen-activated protein kinase in piglets” (2012) 114(1):200-204.

Compounds can vary in their ability to be a KOR agonist and MOR agonist compared to Salvinorin A. As used herein, the term “stronger” in relation to Salvinorin A means having a higher affinity to a target receptor than Salvinorin A and/or having higher efficacy at producing a biological response upon binding to a target receptor than Salvinorin A. In some embodiments, a compound described herein, or a pharmaceutically acceptable salt thereof, can be a stronger KOR agonist than Salvinorin A. In some embodiments, a compound described herein, or a pharmaceutically acceptable salt thereof, can be a stronger MOR agonist than Salvinorin A. In some embodiments, a compound described herein, or a pharmaceutically acceptable salt thereof, can be a stronger MOR agonist and KOR agonist than Salvinorin A. In some embodiments, a compound described herein, or a pharmaceutically acceptable salt thereof, can have substantial MOR agonist activity compared to Salvinorin A. In some embodiments, a compound described herein, or a pharmaceutically acceptable salt thereof, can have substantial KOR agonist activity compared to Salvinorin A. As used herein, “substantial” agonist activity is where a compound described herein, or a pharmaceutically acceptable salt thereof, has agonist activity that is 25%, 30%, 35%, 400/6, 45%, 50%, 55%, 60%, 65%, 70%, 75% or greater than 75% than the compound to which it is being compared, such as Salvinorin A. In some embodiments, a compound described herein, or a pharmaceutically acceptable salt thereof, can have similar or higher affinity than Salvinorin A and lower efficacy than Salvinorin A for MOR and/or KOR Those skilled in the art would understand that the affinity and/or efficacy of a compound for MOR or KOR agonism can be determined using methods known in the art, such as assays described herein.

Compounds can vary in their bias toward a G protein coupled receptor (GPCR) initiated pathway or a beta-arrestin pathway. In some embodiments, the MOR signaling of a compound described herein, or a pharmaceutically acceptable salt thereof, can be biased toward a GPCR initiated pathway. In other embodiments, the MOR signaling of a compound described herein, or a pharmaceutically acceptable salt thereof, can be biased toward a beta-arrestin pathway. In further embodiments, the MOR signaling of a compound described herein, or a pharmaceutically acceptable salt thereof, can be balanced between a beta-arrestin and a GPCR initiated pathway. In other embodiments, the KOR signaling of a compound described herein, or a pharmaceutically acceptable salt thereof, can be balanced between a beta-arrestin pathway and GPCR initiated pathway. In still other embodiments, the KOR signaling of a compound described herein, or a pharmaceutically acceptable salt thereof, can be biased toward a GPCR initiated pathway. In yet still other embodiments, the KOR signaling of a compound described herein, or a pharmaceutically acceptable salt thereof, can be biased toward a beta-arrestin pathway.

Compounds can vary in their affinity (K₁) and efficacy (EC₅₀) for KOR or MOR. In some embodiments, a compound described herein, or a pharmaceutically acceptable salt thereof, can have both high affinity and high efficacy for KOR and/or MOR. In other embodiments, a compound described herein, or a pharmaceutically acceptable salt thereof, can have high affinity but low efficacy for KOR and/or MOR. In still other embodiments, a compound described herein, or a pharmaceutically acceptable salt thereof, can have low affinity but high efficacy for KOR and/or MOR. Methods for determining affinity and efficacy are known to those skilled in the art. In some embodiments of this paragraph, the affinity and efficacy of a compound described herein, or a pharmaceutically acceptable salt thereof, is compared to Salvinorin A using a method known to those skilled in the art.

Some embodiments described herein relate to a method for treating a subject having a disease or disorder described herein that can include administering an effective amount of a compound described herein (such as a compound of Formula I, Formula II and/or Formula III, or a pharmaceutically acceptable salt of any of the foregoing) or a pharmaceutical composition that includes a compound described herein (such as a compound of Formula I, Formula II, and/or Formula III, or a pharmaceutically acceptable salt of any of the foregoing) to the subject to alleviate at least one symptom of the disease or disorder (such as 1, 2 or 3 symptoms). Other embodiments described herein relate to the use of an effective amount of a compound described herein (such as a compound of Formula I, Formula II, and/or Formula III, or a pharmaceutically acceptable salt of any of the foregoing) or a pharmaceutical composition that includes a compound described herein (such as a compound of Formula I, Formula II, and/or Formula III, or a pharmaceutically acceptable salt of any of the foregoing) in the manufacture of a medicament for treating a disease or disorder described herein. Still other embodiments described herein relate to an effective amount of a compound described herein (such as a compound of Formula I, Formula II, and/or Formula III, or a pharmaceutically acceptable salt of any of the foregoing) or a pharmaceutical composition that includes a compound described herein (such as a compound of Formula I, Formula II, and/or Formula III, or a pharmaceutically acceptable salt of any of the foregoing) for treating a disease or condition described herein.

Some embodiments described herein relate to a method for treating a subject having a disease or disorder described herein that can include contacting a KOR and/or a MOR receptor in a subject with an effective amount of a compound described herein (such as a compound of Formula I, Formula II, and/or Formula III, or a pharmaceutically acceptable salt of any of the foregoing). Other embodiments described herein relate to the use of an effective amount of a compound described herein (such as a compound of Formula I, Formula II, and/or Formula III, or a pharmaceutically acceptable salt of any of the foregoing) in the manufacture of a medicament for contacting a KOR and/or a MOR receptor in a subject having a disease or disorder described herein. Still other embodiments described herein relate to an effective amount of a compound described herein (such as a compound of Formula I, Formula II, and/or Formula III, or a pharmaceutically acceptable salt of any of the foregoing) for contacting a KOR and/or a MOR receptor in a subject having a disease or disorder described herein.

Some embodiments described herein relate to a method for ameliorating one or more symptom (such as 1, 2 or 3 symptoms) of a disease or disorder described herein that can include administering an effective amount of a compound described herein (such as a compound of Formula I, Formula II, and/or Formula III, or a pharmaceutically acceptable salt of any of the foregoing) to a subject suffering from the disease or disorder; and can also include contacting a KOR and/or a MOR receptor with an effective amount of a compound described herein (such as a compound of Formula I, Formula II, and/or Formula III, or a pharmaceutically acceptable salt of any of the foregoing). Other embodiments described herein relate to the use of an effective amount of a compound described herein (such as a compound of Formula I, Formula II, and/or Formula III, or a pharmaceutically acceptable salt of any of the foregoing) in the manufacture of a medicament for ameliorating one or more symptom (such as 1, 2 or 3 symptoms) of a disease or disorder described herein; or, in the manufacture of a medicament for ameliorating one or more symptom (such as 1, 2 or 3 symptoms) of a disease or disorder described herein, wherein the use comprises contacting a KOR and/or a MOR receptor with the medicament. Still other embodiments described herein relate to an effective amount of a compound described herein (such as a compound of Formula I, Formula II, and/or Formula III, or a pharmaceutically acceptable salt of any of the foregoing) for ameliorating one or more symptom (such as 1, 2 or 3 symptoms) of a disease or disorder described herein by contacting a KOR and/or a MOR receptor.

In some embodiments, the compounds described herein may be used to treat or inhibit any disease or disorder or side effect of a drug that can be treated with a KOR agonist and/or MOR agonist. In some embodiments, the disease or disorder or side effect can be selected from stroke, ischemia, hypoxia, hypoxic-ischemic encephalopathy. Raynaud's disease, Alzheimer's disease, migraines, headaches, pain, myocardial infarction, cardiac arrest, acute respiratory distress syndrome, acute lung injury, conditions associated with pruritus, at risk for or experiencing excess or inappropriate clot formation, inflammation, conditions associated with inflammation, edema, conditions associated with edema, HIV-induced neuroinflammation, emesis, conditions associated with emesis, hemorrhage, interstitial lung disease, hemorrhagic stroke, ischemic stroke, diseases associated with induction of anesthesia including spine injury and neural injury, spinal injury, neural injury, hypoxic pulmonary hypertension, multiple sclerosis, addiction or substance use disorder, post-traumatic cartilage regeneration, a psychiatric disorder, a mood disorder, mania, bipolar disorder, an autism spectrum disorder, irritable bowel disease, a circulatory disease or disorder, a cardiac disease, a brain disease or injury, traumatic brain injury, chronic traumatic encephalopathy, aneurysm, a pulmonary disease or disorder, a spinal cord disease or disorder, a disease related to dopamine, intestinal motility including diarrhea, rheumatism, obesity, stress, cognitive impairment, reduction in side effects related to chemotherapy or apoptosis or another type of programmed cell death (for example, PANoptosis, pyroptosis, etc.) that was promoted or induced by drugs or a disease or a disorder, epilepsy, seizures, diuresis, cerebral edema, intracerebral hemorrhage, subarachnoid hemorrhage, intraventricular hemorrhage, dementia, allergic disease, asthma, a respiratory virus infection or one or more complications from a respiratory virus infection, including but not limited to a coronavirus, for example COVID-19 (e.g., damage to organs or vasculature from endothelial cell damage), chronic pruritus including but not limited to cancer related pruritus, brachioradial pruritus, post herpetic pruritus, aquagenic pruritus, uremic pruritus prurigo nodularis, idiopathic pruritus, urticaria neuropathic pruritus, pruritus induced by multiple sclerosis, HIV protease inhibitor, hepatitis C chemotherapy, burn, chronic cirrhosis, atopic dermatitis, lichen simplex chronicus, psoriasis, primary sclerosing cholangitis, Hodgkin's lymphoma, psychiatric causes, primary biliary cholangitis or polycythemia vera, chronic cough, including refractory chronic cough and chronic cough caused by COPD, emphysema, chronic bronchitis, GERD, heart failure, idiopathic nonspecific interstitial pneumonia, bronchiectasis, hyper-sensitivity pneumonitis, asthma, lung cancer, idiopathic pulmonary fibrosis, unclassified idiopathic interstitial pneumonia, autoimmune interstitial lung disease, other interstitial lung diseases (e.g., sarcoidosis), post-nasal drip or tobacco smoke/usage. In specific embodiments, the disease or disorder is allergic rhinitis (See Shou et al., FEBS Open Bio 11:2166-2173 (2021)), diseases caused by disruption of endothelial mitochondrial function (See Dong et al., Exp. Neurology 322:113045 (2019)) including a disease that causes endothelial damage, dysfunction or apoptosis or another type of programmed cell death (PANoptosis, pyroptosis, etc.), vascular damage, vasoplegia or conditions associated with vasoplegia, inflammation, pneumonia, myocardial injury or damage (including but not limited to myocardial ischemia, myocardial infarction, and myocarditis), constriction of blood vessels, macrophage and or neutrophil infiltration, and vascular disease caused by a respiratory virus such as COVID-19. In some embodiments, the disease is allergic rhinitis, a disease caused by disruption of endothelial mitochondrial function including vascular disease caused by a respiratory virus such as COVID-19, or asthma or another allergic disease (See Siddigi et al., Trends in Cardiovascular Medicine 31:1-5 (2021)), or asthma and other allergic diseases (See Rossi et al., Front. Pharmacol. 7:525 (2017)).

Where the disease or disorder is COVID-19, particularly when the subject is at high risk (See, e.g., cdc.gov/coronavirus/2019-ncov/need-extra-precautions/people-with-medical-conditions.html), has an 02 saturation of <95% or has shortness of breath, a compound described herein, or a pharmaceutically acceptable salt thereof, can be co-administered with other COVID-19 therapeutics, including, but not limited to, Paxlovid (Nirmatrelvir), molnupiravir, dexamethasone, remdesivir, convalescent plasma, monoclonal antibodies to treat COVID or another respiratory virus (e.g., sotrovimab, bamlanivimab, etesevimab, Casirivimab, imdevimab), fluvoxamine, and medications for subjects in the hospital, on oxygen, or intubated on a ventilator (e.g., benzodiazepines, propofol, dexmedetomidine, other sedatives, fentanyl or other pain controllers).

Some embodiments described herein relate to a method for activating a KOR and/or a MOR receptor that can include contacting a cell with an effective amount of a compound described herein (such as a compound of Formula I, Formula II, and/or Formula III, or a pharmaceutically acceptable salt of any of the foregoing). Other embodiments described herein relate to the use of an effective amount of a compound described herein (such as a compound of Formula I, Formula II, and/or Formula III, or a pharmaceutically acceptable salt of any of the foregoing) in the manufacture of a medicament for activating a KOR and/or a MOR receptor in a cell. Still other embodiments described herein relate to an effective amount of a compound described herein (such as a compound of Formula I, Formula II, and/or Formula III, or a pharmaceutically acceptable salt of any of the foregoing) for activating a KOR and/or a MOR receptor in a cell.

Some embodiments described herein relate to a method for modulating the activity of a KOR and/or a MOR receptor that can include contacting a cell with an effective amount of a compound described herein (such as a compound of Formula I, Formula II, and/or Formula III, or a pharmaceutically acceptable salt of any of the foregoing). Other embodiments described herein relate to the use of an effective amount of a compound described herein (such as a compound of Formula I, Formula II, and/or Formula III, or a pharmaceutically acceptable salt of any of the foregoing) in the manufacture of a medicament for modulating the activity of a KOR and/or a MOR receptor in a cell. Still other embodiments described herein relate to an effective amount of a compound described herein (such as a compound of Formula I, Formula II, and/or Formula III, or a pharmaceutically acceptable salt of any of the foregoing) for modulating the activity of a KOR and/or a MOR receptor in a cell.

In some embodiments, more than one of the compounds described herein (including a pharmaceutically acceptable salt thereof), e.g., at least two compounds from Formula I, or a pharmaceutically acceptable salt thereof, at least two compounds from Formula II, or a pharmaceutically acceptable salt thereof, at least two compounds from Formula III, or a pharmaceutically acceptable salt thereof, or at least two compounds from Formula I, Formula II, or Formula III, or a pharmaceutically acceptable salt thereof, can be administered to a subject.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, that elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound is the amount that results in: (a) the reduction, alleviation or disappearance of one or more symptoms caused by the disease or disorder, (b) the elimination of the disease or disorder, and/or (c) long-term stabilization of the disease or disorder.

The amount of the compound of Formula I, Formula II or Formula III, or a pharmaceutically acceptable salt thereof, required for use in treatment will vary not only with the particular compound or salt selected but also with the route of administration, the nature and/or symptoms of the disease or condition being treated and the age and condition of the subject and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, which is the dosage levels necessary to achieve the desired result, can be accomplished by one skilled in the art using routine methods, for example, human clinical trials, in vivo studies and in vitro studies. For example, useful dosages of a compound of Formula I. Formula II, or Formula III, or pharmaceutically acceptable salts thereof, can be determined by comparing their in vitro activity and in vivo activity in animal models. Such comparison can be done by comparison against an established compound, such as Salvinorin A.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However. HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual subject. A program comparable to that discussed above may be used in veterinary medicine.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated. The alkyl group may also be a medium size alkyl having 1 to 12 carbon atoms. The alkyl group could also be a lower alkyl having 1 to 6 carbon atoms. An alkyl group may be substituted or unsubstituted.

As used herein, the term “methyl” refers to a —CH₃ group. Those skilled in the art understand that methyl can be abbreviated as Me.

As used herein, “cycloalkyl” refers to a completely saturated (no double or triple bonds) mono- or multi-cyclic hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Typical mono-cycloalkyl groups include, but are in no way limited to, cyclopropyl, cyclobutyl, cyclopentyl, cyclohexyl, cycloheptyl, and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl, and norbornanyl; and examples of spiro cycloalkyl groups include spiro[3.3]heptane and spiro[4.5]decane.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen (N), oxygen (O) and sulfur (S). The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl functionalities, so as to make the definition include oxo-systems. When composed of two or more rings, the rings may be joined together in a fused, bridged or Spiro fashion. As used herein, the term “fused” refers to two rings which have two atoms and one bond in common. As used herein, the term “bridged heterocyclyl” refers to compounds wherein the heterocyclyl contains a linkage of one or more atoms connecting non-adjacent atoms. As used herein, the term “spiro” refers to two rings which have one atom in common and the two rings are not linked by a bridge. Heterocyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). Additionally, any nitrogens in a heterocyclyl may be quaternized. Heterocyclyl groups may be unsubstituted or substituted. Examples of such “heterocyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, the term “hydroxy” refers to an —OH group.

As used herein, “alkoxy” refers to the Formula —OR, wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) as defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, the term “acetoxy” refers to an —OCOCH₃ group. Those skilled in the art understand that acetoxy can be abbreviated AcO or OAc.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

The term “halogen” as used herein means any one of the radio-stable atoms of column 7 of the Periodic Table of the Elements, such as fluorine (F), chlorine (Cl), bromine (Br) and iodine (1).

The term “ester” refers to a “—C(═O)OR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An ester may be substituted or unsubstituted.

A “nitro” group refers to an “—NO₂” group.

A “sulfonyl” group refers to an “SO₂R” group in which R can be hydrogen, an alkyl, an alkenyl, an alkynyl, a cycloalkyl, a cycloalkenyl, aryl, heteroaryl, heterocyclyl, cycloalkyl (alkyl), aryl(alkyl), heteroaryl(alkyl) or heterocyclyl(alkyl). A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

The term “amino” as used herein refers to a —NH—, group.

A “mono-substituted amino” group refers to a “—NHR” group in which R can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. A mono-substituted amino may be substituted or unsubstituted. Examples of monosubstituted amino groups include but are not limited to —NH(methyl), —NH(phenyl) and the like.

A “di-substituted amino” group refers to a “—NR_{A}R_{B}” group in which Li and Ra can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. A di-substituted amino may be substituted or unsubstituted. Examples of di-substituted amino groups include, but are not limited to, —N(methyl)2, —N(phenyl)(methyl), —N(ethyl)(methyl) and the like.

Where the numbers of substituents is not specified (e.g., haloalkyl), there may be one or more substituents present. For example “haloalkyl” may include one or more of the same or different halogens.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof, and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### REFERENCES

Akins, N. S., Mishra, N., Harris, H. M., Dudhipala, N., Kim, S. J., Keasling, A. W., Majumdar, S., Zjawiony, J. K., Paris, J. J., Ashpole, N. M., & Le, H. V. (2022). 6,5-Fused Ring, C2-Salvinorin Ester, Dual Kappa and Mu Opioid Receptor Agonists as Analgesics Devoid of Anxiogenic Effects. ChemMedChem, 17(7), e202100684. https://doi.org/10.1002/cmdc.202100684
Beck, T. C., Hapstack, M. A., Beck, K. R. & Dix, T. A. (2019). Therapeutic Potential of Kappa Opioid Agonists. Pharmaceuticals (Basel, Switzerland), 12(2), 95. https://doi.org/10.3390/ph12020095
Beguin C et al., Bioorganic & Medicinal Chemistry Letters 16:4679-4685 (2006).
Beguin C et al., J. Pharmacol. Exp. Ther. 324:188-195 (2008).
Berman Y E et al., J. Org. Chem. 74:2589-2591 (2009).
Bhowmik S et al., Nature Communications doi.org/10.1038/s41467-021-23736-2 (2021).
Bonaventura J. et al., Mol. Psychiatry doi.org/10.1038/s41380-021-01093-2 (2021).
Bonaventura A. et al., Nat. Reviews/Immunology 21:319-329 (2021).
Brito-da-Costa A M et al., Pharmaceuticals 14:116 (2021).
Brust, T. F., Morgenweck, J., Kim, S. A., Rose, J. H., Locke, J. L., Schmid, C. L., Zhou, L., Stahl, E. L., Cameron, M D., Scarry, S. M., Aube, J., Jones, S. R, Martin, T. J., & Bohn, L. M. (2016). Biased agonists of the kappa opioid receptor suppress pain and itch without causing sedation or dysphoria. Science signaling, 9(456), rail 7. https://doi.org/10.1126/scisignal.aai8441
Butelman E R and Kreek M J, Front. Pharmacol. 6:190 (2015).
Chakraborty S and Majumdar S. Biochemistry DOI: 10.1021/acs.biochem.0c00629 (2020).
Chunhua C et al., Transl Perioper Pain Med. 1:27-34 (2014).
Cichon, J., Liu, R. & Le, H. V. (2022). Therapeutic Potential of Salvinorin A and Its Analogues in Various Neurological Disorders. Translational perioperative and pain medicine, 9(2), 452-457.
Crowley R S et al., ACS Chem. Neurosci DOI: 10.1021/acschemneuro.0c00191 (2020).
Dalefield, M. L., Scouller, B., Bibi, R., & Kivell, B. M. (2022). The Kappa Opioid Receptor: A Promising Therapeutic Target for Multiple Pathologies. Frontiers in pharmacology, 13, 837671. https::/doi.org/10.3389/fphar.2022.837671
Dong et al., Exp. Neurology 322:113045 (2019).
Grothusen J, Transl Perioper & Pain Med 8:337-341 (2021).
Gupta A et al., Proc. Natl. Acad. Sci. USA 113:6041-6046 (2016).
Hernandez-Alvarado R B et al., ACS Chemical Neuroscience doi.org/10.1021/acschemneuro.0c00608 (2020).
Hill Si et al., Nat. Prod. Rep. DOI: 10.1039/d0np00028k (2020).
Hirasawa S et al., Bioorg Med Chem Lett. 28:2770-2772 (2018).
Ibarra, Y E, Doctoral Dissertation, Harvard University (2013).
Jamieson C S et al., Chem. Soc. Rev. DO: 10.1039/d1cs00065a (2021).
Ji F et al., Brain Res. 1490: 95-100 (2013).
Kutrzeba L. et al., Phytochemistry 68:1872-1881 (2007).
Liu, L. J., Yu, J. J., & Xu, X. L. (2018). Kappa-opioid receptor agonist U50448H protects against renal ischemia-reperfusion injury in rats via activating the PI3K/Akt signaling pathway. Acta pharmacologica Sinica, 39(1), 97-106. https://doi.org/10.1038/aps.2017.51
Majer et al., Bioorg. Med. Chem. 22:256 (2014).
Mores K L et al., Front. Pharmacol. 10:407 (2019).
Paton K F et al., Front. Neurosci. 14:765 (2020).
Paton, K. F., Atigari, D. V., Kaska, S., Prisinzano, T., & Kivell, B. M. (2020). Strategies for Developing K Opioid Receptor Agonists for the Treatment of Pain with Fewer Side Effects. The Journal of pharmacology and experimental therapeutics, 375(2), 332-348. https://doi.org/10.1124/jpet. 120.000134
Pelot K A. et al., The Plant Journal 89:885-897 (2017).
Polepally P R et al., Eur. J. Med. Chem. 85:818-829(2014).
Prisinzano T and Rothman R B., Chem. Rev. 108:1732-1743 (2008).
Remington: The Science and Practice of Pharmacy, Lippincot Williams & Wilkins, 2006. 2393 pages.
Riley A P et al., J. Med. Chem. 57:10464-10475 (2014).
Roach J J and Shenvi R A, Bioorg Med Chem Lett. 28:1436-1445 (2018).
Rossi et al., Front. Pharmacol. 7:525 (2017).
Roth, B. L., Baner, K., Westkaemper, R., Siebert, D., Rice, K. C., Steinberg, S., Ernsberger, P., & Rothman, R. B. (2002). Salvinorin A: a potent naturally occurring nonnitrogenous kappa opioid selective agonist. Proceedings of the National Academy of Sciences q/the United States of America, 99(18), 11934-11939. https://doi.org/10.1073/pnas.182234399
Salaga M et al., J. Pharmacol. Exp. Ther. 350:69-78 (2014).
Sagala M et al., Br. J. Pharmacol. 172:4331-4341 (2015).
Sharma et al., Bioorg. Med. Chem. 18:6886 (2010).
Shenvi et al., ACS Cent. Sci. 3:1329 (2017).
Shenvi et al., Bioorg. Med. Chem. Lett. 28:2770 (2018).
Shou et al., FEBS Open Bio 11:2166-2173 (2021).
Siddigi et al., Trends in Cardiovascular Medicine 31:1-5 (2021).
Su, D., Riley, J., Kiessling, W. J., Armstead, W. M., & Liu, R. (2011). Salvinorin A produces cerebrovasodilation through activation of nitric oxide synthase, x receptor, and adenosine triphosphate-sensitive potassium channel. Anesthesiology. 114(2), 374-379. https://doi.org/10.1097/ALN.0b013e318204e029
Su, D., Riley, J., Armstead, W. M., & Liu, R (2012). Salvinorin A pretreatment preserves cerebrovascular autoregulation after brain hypoxic/ischemic injury via extracellular signal-regulated kinase/mitogen-activated protein kinase in piglets. Anesthesia and analgesia, 114(1), 200-204. https://doi.org/10.1213/ANE.0b013e31823a5d36
Sun J. et al., Brain Research 1719:64-70 (2019).
Wang, Y et al., Acta Pharmacologica Sinica 31:1065-1070 (2010).
White K L, et al., J Pharmacol Exp Ther 352:98-109 (2015).
Xu, J et al., Neurochem Int. 137 (2020).
Zeng S et al., International Immunopharmacology 90:107221 (2021).
Zimdars P et al., Chem. Eur. J. 27:7968-7973 (2021).
U.S. Pat. No. 7,687,638.
U.S. Pat. No. 7,728,001.
PCT Patent Application Publication WO 2005/089745 A1.
PCT Patent Application Publication WO 2015/143429 A2.
US Patent Application Publication 2013/0102659 A1.
US Patent Application Publication 2020/0131162 A1.
German Patent Application Publication DE10356406A1.

### Examples

Additional embodiments are disclosed in further detail in the following examples, which are not in any way intended to limit the scope of the claims.

TABLE 1
Experimental Data of Synthesis

| | | |
|---|---|---|
| Starting | | |
| Material | Product | |
| Amount | Yield | Comments |

Synthesis of allylic alcohol (FIG. 2B)

| | | |
|---|---|---|
| 4 g | H-3 | Freshly activated Mg turnings, anhydrous THF generated; |
| H-2 | | catalytic 1,2-dibromoethane (0.25 eq.) added, reflux for |
| | | 6 h. Confirmed activity of Grignard reagent by reacting |
| | | with benzaldehyde; desired adduct formed. |
| 20 g | H-3 | Freshly activated Mg turnings, anhydrous THF generated, |
| H-2 | | catalytic 1,2-dibromoethane (0.20 eq.); stored at 0° C. |
| | | Grignard reagent generated (confirmed by benaldehyde |
| | | reaction). |
| 20 g | 85 mL | Freshly activated Mg turnings, anhydrous THF generated, |
| H-2 | of | catalytic 1,2-dibromoethane (0.15 eq.). Starting material |
| | Grignard | consumed/Mg turnings consumed. |
| | reagent | |
| | (~0.5M) | |
| | (H-3) | |
| 1.0 g | 250 mg | ELSD-MS & H NMR consistent. |
| H-4 | H-5 | Grignard reagent (3 eq.) used. |
| | (10%) | |
| 45 g | 200 mL | Freshly activated Mg turnings, anhydrous THF generated, |
| H-2 | of | catalytic 1,2-dibromo ethane (0.15 eq.). Starting material |
| | Grignard | consumed/Mg turnings consumed. |
| | reagent | |
| | (~0.5M) | |
| | (H-3) | |
| 11.0 g | 3.50 g | Purified by chromatography; consistent by ELSD-MS & |
| H-4 | H-5 | H NMR analyses. Used 2 Grignard reagent (2 eq.), |
| | (13%) | recovered 5.0 g of H-4. |

Sythesis of O6C-20-nor-SalB (FIG. 4)

| | | |
|---|---|---|
| 10 mg | 2 mg | Used racemic material recovered from SFC purification; |
| | (impure) | Correct mass ion observed UPLC-MS. |
| 30 mg | 20 mg | |
| | (crude) | H NMR & UPLC-MS consistent. |

Synthesis of Target 1 (FIG. 5B)

| | | |
|---|---|---|
| 5 mg | ~3.5 mg | Combined and purified by column chromatography and |
| 10 mg | Target | then reverse phase chromatography; H NMR & |
| | 1 | UPLC-MS consitent |

Synthesis of Target 3 (FIG. 6B)

| | | |
|---|---|---|
| 5 mg | ~3.5 mg | Correct mass ion observed in UPLC-MS of |
| | Target | crude product. |
| | 3 | |

Synthesis of Target 21 (FIG. 9A)

| | | |
|---|---|---|
| 1.7 g | 240 mg | Conditions: Toluene (200 vol), 60 C., 4.5 h: Purified by |
| | 12, 14%) | column chromatography; ¹H NMR and UPLC-MS |
| | 110 mg | consistent. |
| | (12a, | |
| | 6%) | |

### Experiment 3.1

The ability of the compounds tested to act as a Kappa opioid receptor (KOR) agonist was evaluated using the DiscoverX cAMP Hunter eXpress KOR GPCR Assay kit. A summary of the compounds tested is provided in Table 2. The DiscoverX cAMP Hunter eXpress KOR GPCR Assay kit is designed to detect inhibition of intracellular cyclic AMP (cAMP) production in response to agonist stimulation of the Kappa opioid receptor. cAMP production was stimulated by treatment with a constant concentration of Forskolin in parallel with a concentration response of Salvinorin A or the compounds being tested. Agonist binding to KOR is predicted to inhibit the production of cAMP in a concentration-dependent manner. cAMP production was measured in the presence of varying concentrations of Salvinorin A or the compounds being tested and used to determine the half maximal effective concentration (EC₅₀) of each respective KOR agonist.

TABLE 2
Compounds Submitted to Biological Testing

| Name | Structure | Sample No. |
|---|---|---|
| Salvinorin A | 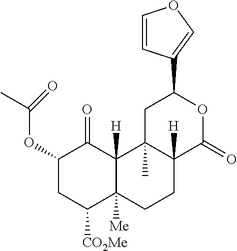 | SalA |
| O6C-20- nor-SalA racemic mixture | 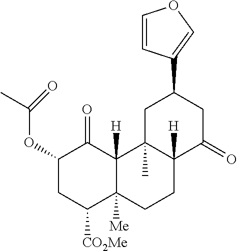 | ALB-230937 |
| O6C-20-nor-SalA | | ALB-230936 |
| Enantiomer | | |
| A | | |
| O6C-20-nor-SalA | | ALB-230932 |
| Enantiomer | | |
| B | | |
| 2-cpi-O6C- 20-nor-SalA | 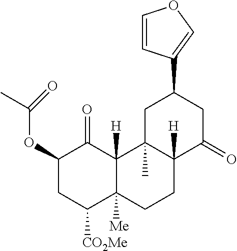 | ALB-230935 |
| O6C-20-nor-SalB | 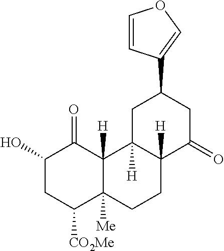 | ALB-231274 |
| Target 1 | 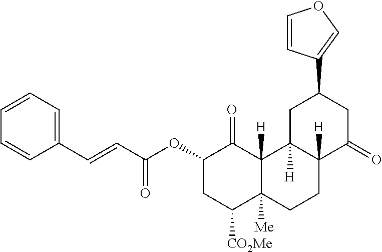 | ALB-231360 |
| Target 3 | 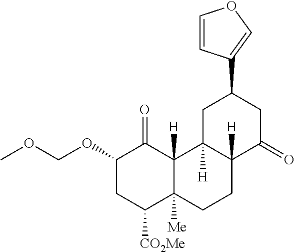 | ALB-231273 |
| Target 21 | 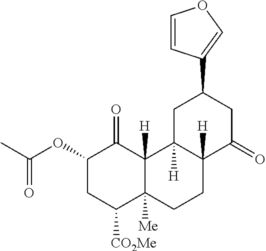 | ALB-231936 |

Agonist concentration responses were performed in 96-well plate format using the DiscoverX cAMP Hunter eXpress KOR GPCR Assay kit. In this assay, 15 μM Forskolin was sufficient to induce production of cAMP to a level that falls within the dynamic range of the kit-provided standard curve. Both Salvinorin A and ALB-230937 effectively inhibited 100% of cAMP production at the lowest concentration tested, 0.1 nM. Due to the high potency of both Salvinorin A and ALB-230937, a definitive EC₅₀ could not be generated due to the lack of a complete dose-response curve, but it can be reasoned that the EC₅₀ values for each agonist are <0.1 nM. ALB-231360 had an EC₅₀ of ˜46 μM. These results confirm that the DiscoverX cAMP Hunter eXpress KOR GPCR Assay kit is functioning as designed and that ALB-230937 acts as a potent KOR agonist.

### Experiment 3.2

The ability of Salvinorin A and the compounds tested to act as a Kappa opioid receptor (KOR) agonist was evaluated using the DiscoverX cAMP Hunter eXpress KOR GPCR Assay kit. The DiscoverX cAMP Hunter eXpress KOR GPCR Assay kit is designed to detect inhibition of intracellular cyclic AMP (cAMP) production in response to agonist stimulation of the Kappa opioid receptor. cAMP production was stimulated by treatment with a constant concentration of Forskolin in parallel with a concentration response of Salvinorin A or the compounds being tested. Agonist binding to KOR is predicted to inhibit the production of cAMP in a concentration-dependent manner. cAMP production was measured in the presence of varying concentrations of Salvinorin A or the compounds being tested and used to determine the half maximal effective concentration (EC₅₀) of each respective KOR agonist.

Agonist concentration responses were performed in 384-well plate format using the DiscoverX cAMP Hunter eXpress KOR GPCR Assay kit. The implementation of automated liquid handling instrumentation such as the Echo555, Mantis, MultiDrop, and BlueWasher significantly increased the throughput of the assay kit in 384-well plate format. In this assay, 15 μM Forskolin was sufficient to induce production of cAMP to a level that falls within the dynamic range of the kit-provided standard curve. Salvinorin A and the compounds being tested were tested in concentration response in this assay. EC₅₀ values were estimated because of the plate handling and negative control cross-contamination issues. Despite the plate handling and control contamination, the potency of Salvinorin A and ALB-230937 determined in experiment 3.2 were consistent with the observations in experiment 3.1, and agonist concentration ranges were confirmed for all 8 analogs. All compounds had data points sufficient for generating EC₅₀ values, outlined in Table 3.

As shown by the data in Table 3, compounds described herein are active in this assay. In Table 3, “A”=EC₅₀≤1 nM; “B”=EC₅₀>1 nM and <100 nM; “C”=EC₅₀>100 nM and ≤1.000 nM; and “D”=EC₅₀>1,000 nM and ≤10,000 nM. These results confirm that the DiscoverX cAMP Hunter eXpress KOR GPCR Assay kit is functioning as designed and confirms that ALB-230937 acts as potent KOR agonist. From this assay, ALB-231273 and ALB-230936 are also shown to be potent KOR agonists, with estimated EC₅₀ values <0.1 nM.

TABLE 3
Results from Experiment 3.2

| | EC₅₀ |
|---|---|
| Sample No. | Potency Score |
| SalA | A |
| ALB-230937 | A |
| ALB-230936 | A |
| ALB-230932 | B |
| ALB-230935 | B |
| ALB-231274 | D |
| ALB-231360 | D |
| ALB-231273 | A |

### Experiment 3.3

The ability of Salvinorin A and the compounds tested to act as a Kappa opioid receptor (KOR) agonist was evaluated using the DiscoverX cAMP Hunter eXpress KOR GPCR Assay kit. The DiscoverX cAMP Hunter eXpress KOR GPCR Assay kit is designed to detect inhibition of intracellular cyclic AMP (CAMP) production in response to agonist stimulation of the Kappa opioid receptor. cAMP production was stimulated by treatment with a constant concentration of Forskolin in parallel with a concentration response of Salvinorin A or the compounds being tested. Agonist binding to KOR is predicted to inhibit the production of cAMP in a concentration-dependent manner. cAMP production was measured in the presence of varying concentrations of Salvinorin A or the compounds being tested and used to determine the half maximal effective concentration (EC %) of each respective KOR agonist.

Agonist concentration responses were performed in 384-well plate format using the DiscoverX cAMP Hunter eXpress KOR GPCR Assay kit. The use of automated liquid handling instrumentation such including the Echo555, Mantis, MultiDrop, and BlueWasher was implemented to further increase the throughput of the assay kit in 384-well plate format. In this assay, 15 μM Forskolin was again sufficient to induce production of cAMP to a level that falls within the dynamic range of the kit-provided standard curve. Salvinorin A and the compounds being tested were tested in concentration response in this assay. EC₅₀ values were calculated using the positive and negative controls included in this assay. Potency of Salvinorin A and ALB-230937 in experiment 3.3 were consistent with EC₅₀ values generated in experiments 3.1 and 3.2. All compounds had data points sufficient for generating EC₅₀ values, which are outlined in Table 4.

As shown by the data in Table 4, compounds described herein are active in this assay. In Table 3. “A”=EC₅₀≤1 nM; “B”=EC₅₀>1 nM and ≤100 nM; “C”=EC₅₀>100 nM and ≤1.000 nM; and “D”=EC₅₀>1,000 nM and ≤10,000 nM. These results confirm that the DiscoverX cAMP Hunter eXpress KOR GPCR Assay kit is functioning as designed and confirms that ALB-230937 act as a potent KOR agonist. In addition, the data provided in Table 4 demonstrate that ALB-231273 and ALB-230936 are also potent KOR agonists, with estimated EC₅₀ values <0.1 nM.

TABLE 4
Results from Experiment 3.3

| | EC₅₀ |
|---|---|
| Sample No. | Potency Score |
| SalA | A |
| ALB-230937 | A |
| ALB-230936 | A |
| ALB-230932 | B |
| ALB-230935 | B |
| ALB-231274 | C |
| ALB-231360 | D |
| ALB-231273 | A |
| ALB-231933 | A |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A compound having Formula I, Formula II, or Formula III, 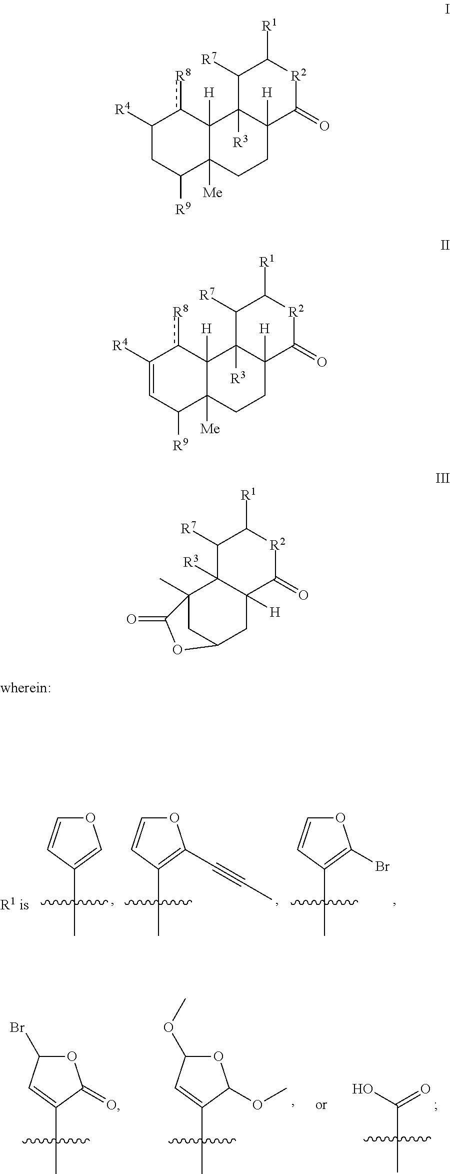
R² is O or CH₂;
R³ is H or CH₃;
R⁴ is selected from the group consisting of OH, 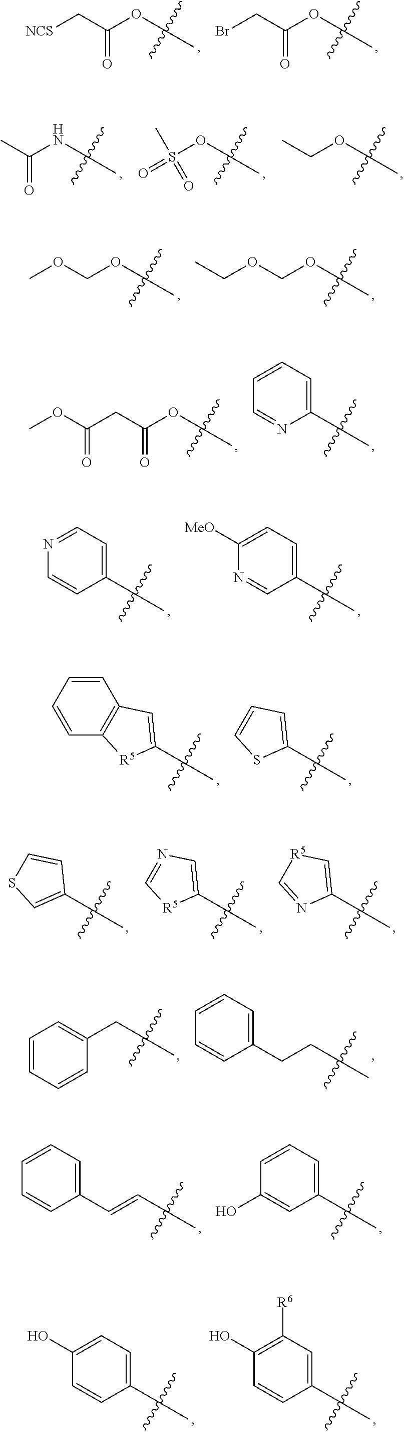 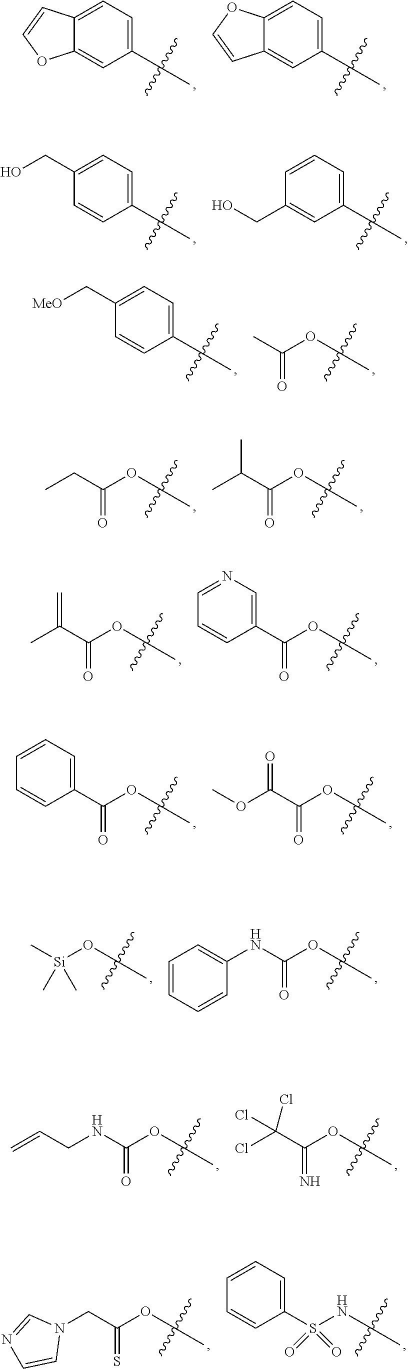 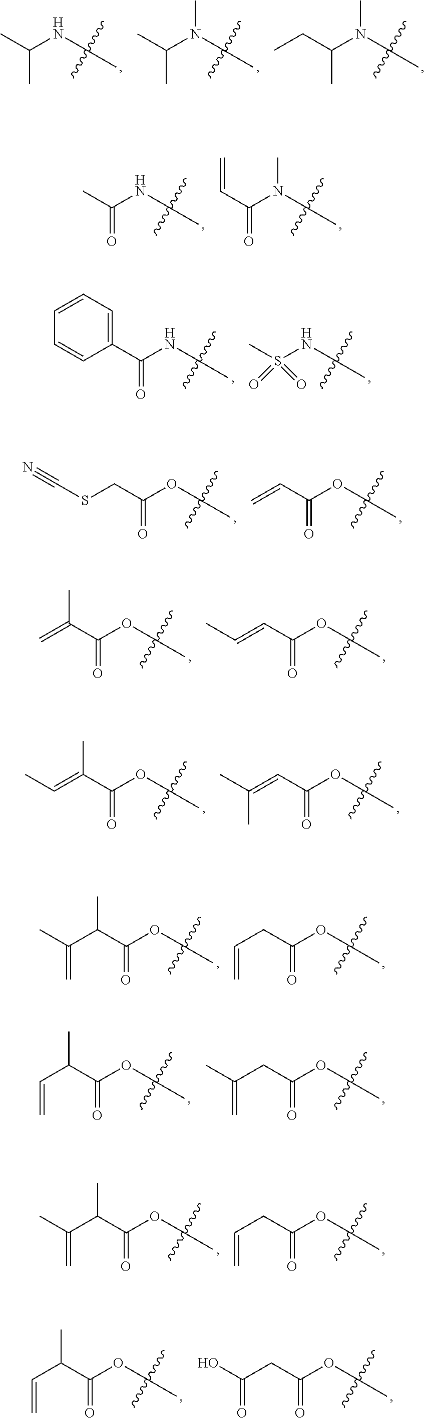 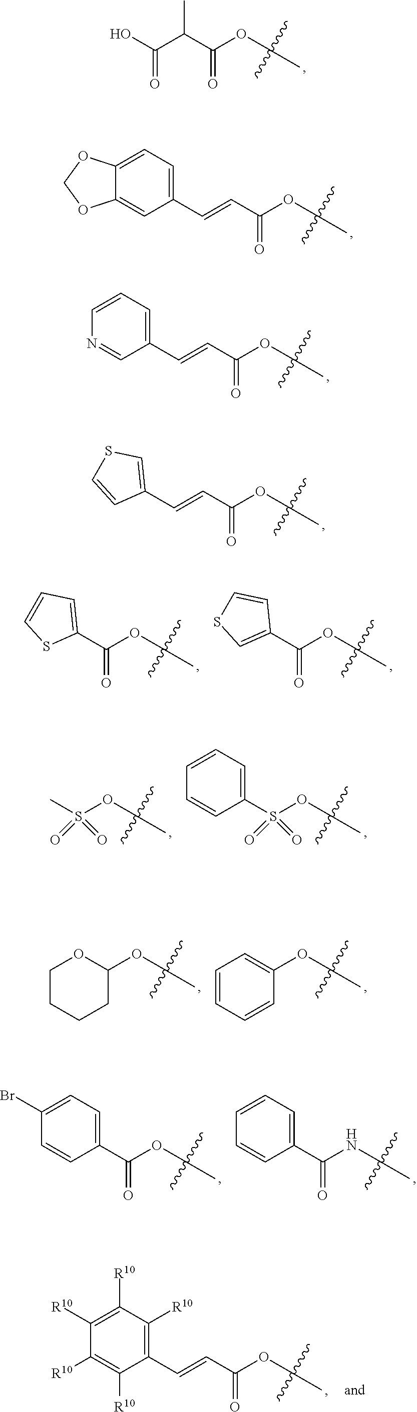 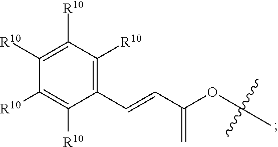
wherein R⁵ is O or S;
R⁶ is F, Cl, or Br; and
each R¹⁰ is independently H, OMe, NO₂, or CF₃;
R⁷ is H or 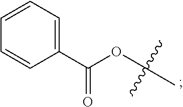 [SMILES: *OC(=O)c1ccccc1]
R⁸ is O, OH, or 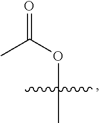 [SMILES: *OC(C)=O]
and [symbol] represents a single bond or a double bond, wherein when
R⁸ is O, the bond represented by [symbol] is a double bond and when R⁸ is OH or 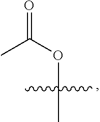 [SMILES: *OC(C)=O]
the bond represented by [symbol] is a single bond;
R⁹ is CO₂Me or CONH₂;
and NCS is N-chlorosuccinimide;
provided that at least one of the following conditions is met: R² is CH₂ or R³ is H; and
provided that the compound is not selected from the group consisting of 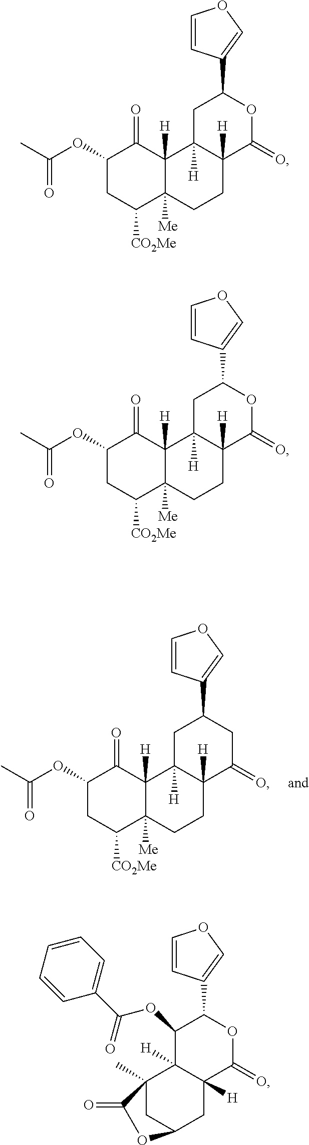
or a pharmaceutically acceptable salt of any of the foregoing.

2. The compound of claim 1, or a pharmaceutically acceptable salt thereof, having Formula I.a:

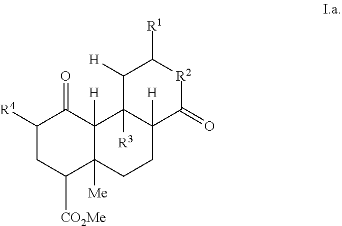

3. The compound of claim 1, or a pharmaceutically acceptable salt thereof, wherein R¹ is

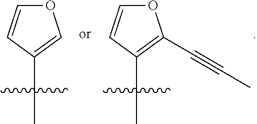

4. The compound of claim 1, or a pharmaceutically acceptable salt thereof, wherein R² is O or CH₂ and/or R³ is H or CH₃ and/or R⁴ comprises a halogen.

5-7. (canceled)

8. The compound of claim 1, or a pharmaceutically acceptable salt thereof, wherein the compound is selected from the group consisting of:

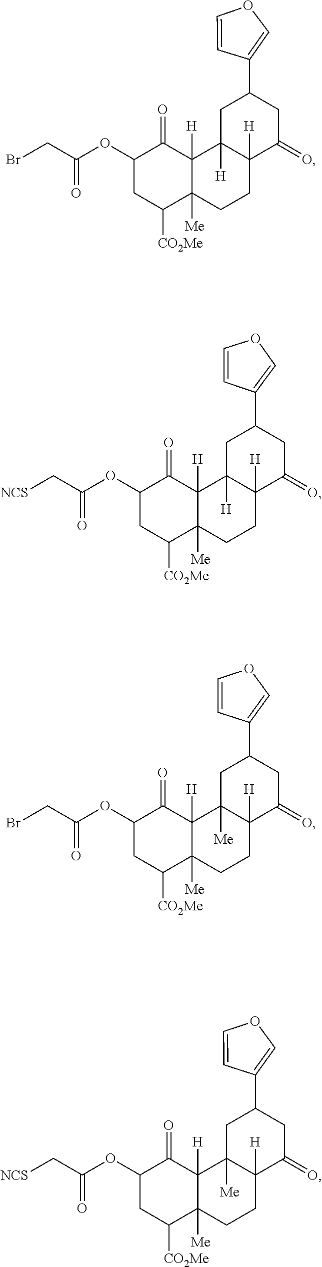

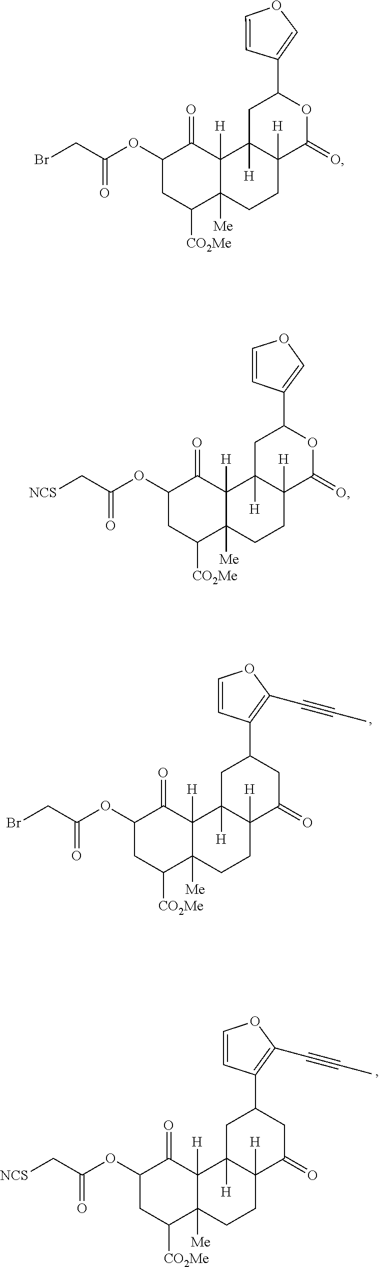

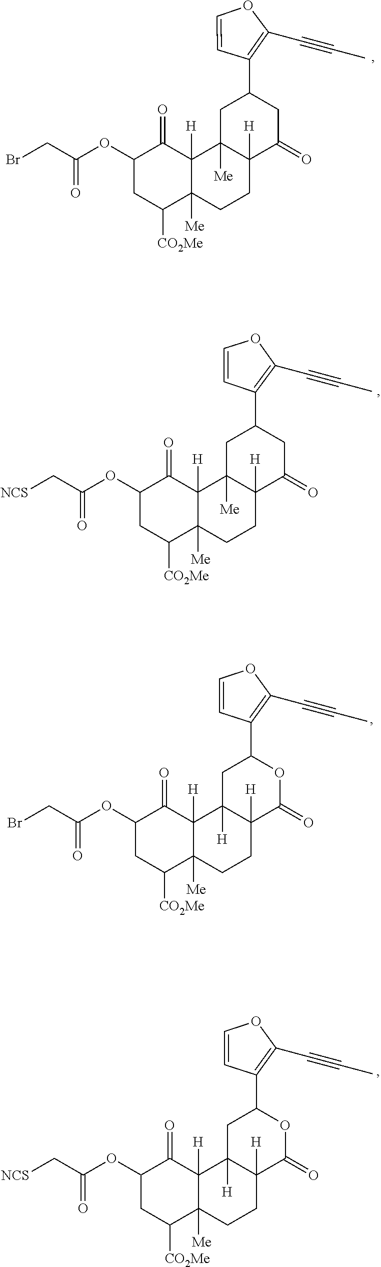

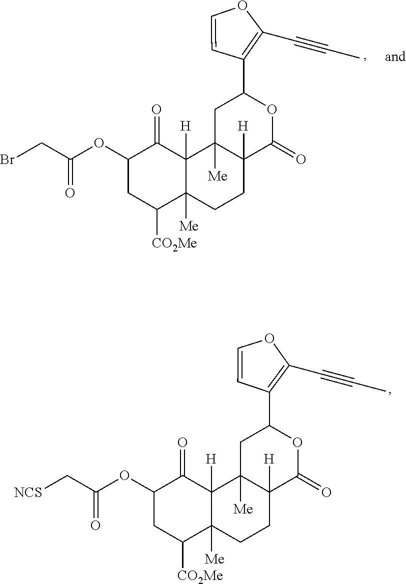

9. (canceled)

10. The compound of claim 1, or a pharmaceutically acceptable salt thereof, wherein R⁴ is selected from the group consisting of: OH,

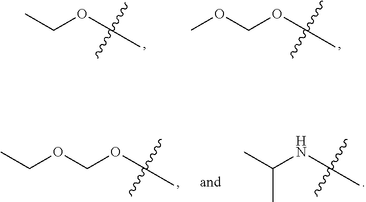

11. The compound of claim 10, or a pharmaceutically acceptable salt thereof, wherein the compound is

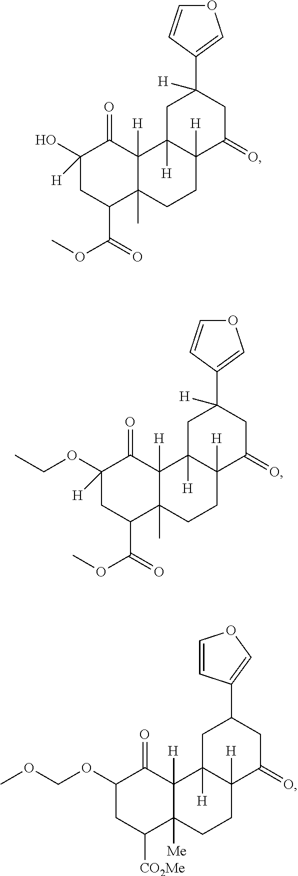

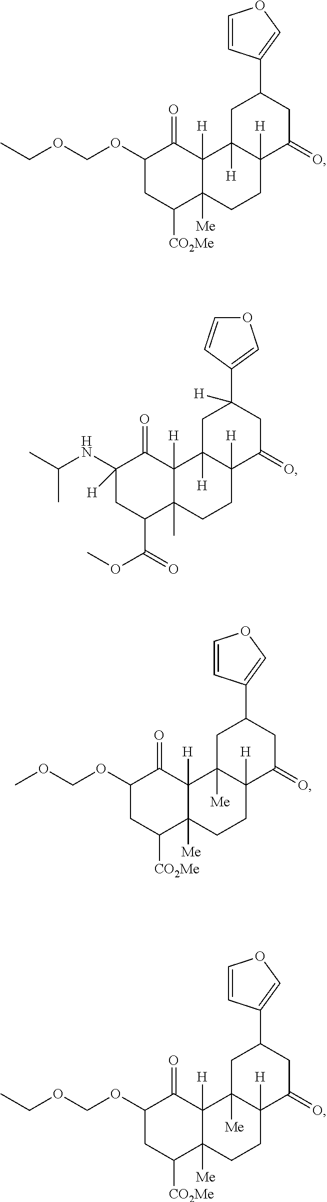

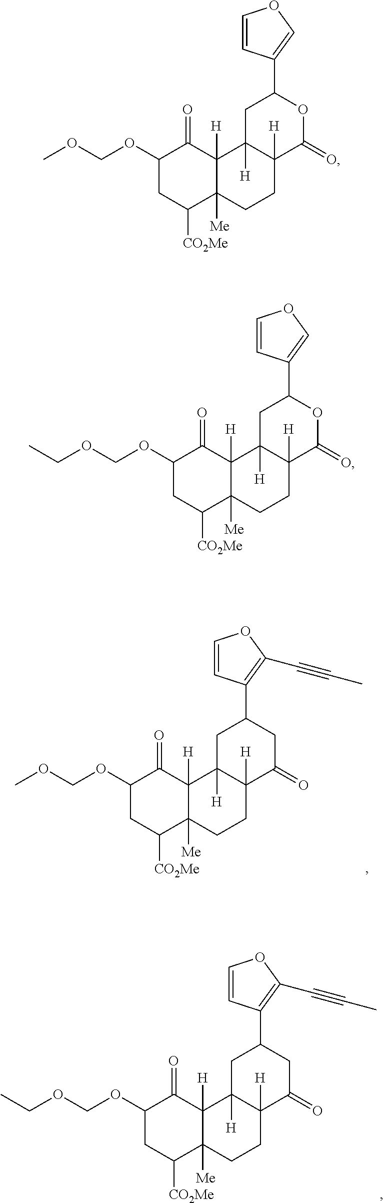

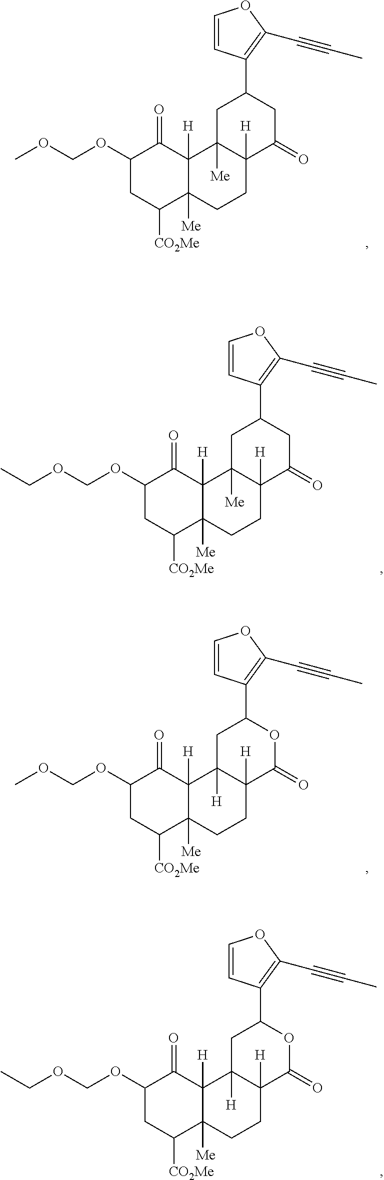

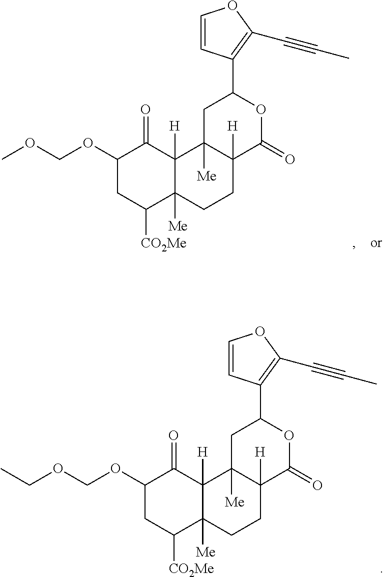

12. The compound of claim 1, or a pharmaceutically acceptable salt thereof, wherein R⁴ comprises an aromatic ring.

13. The compound of claim 12, or a pharmaceutically acceptable salt thereof, wherein R⁴ is selected from the group consisting of:

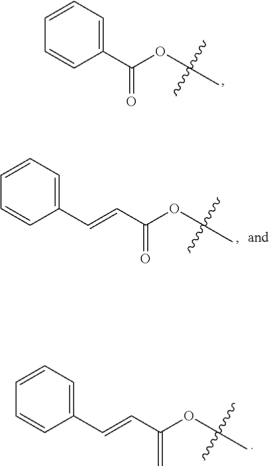 [SMILES: *OC(=O)c1ccccc1 ; *OC(=O)/C=C/c1ccccc1 ; *OC(=C)/C=C/c1ccccc1]

14. The compound of claim 13, or a pharmaceutically acceptable salt thereof, wherein the compound is selected from the group consisting of:

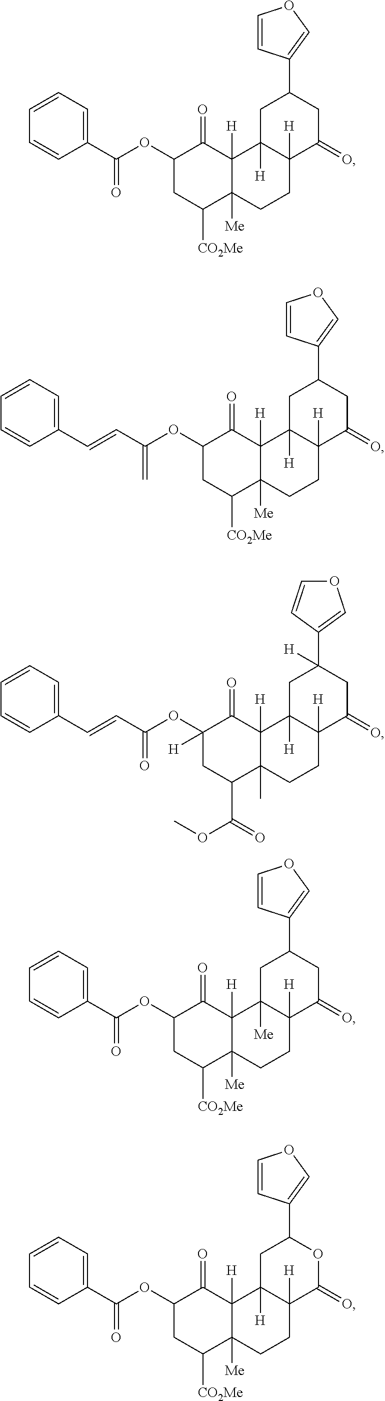

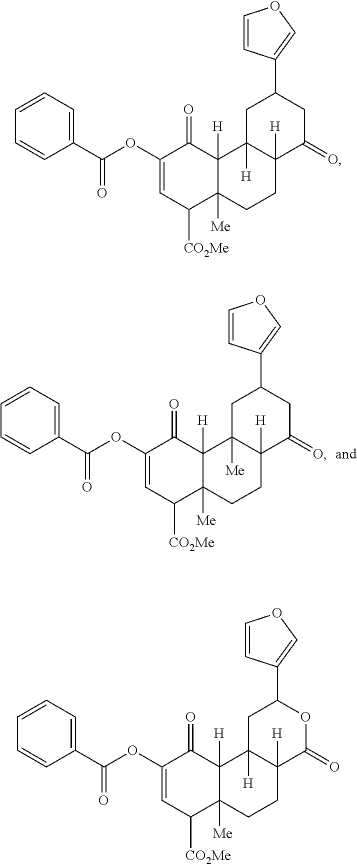

15. (canceled)

16. The compound of claim 1, or a pharmaceutically acceptable salt thereof, wherein R⁴ is selected from the group consisting of:

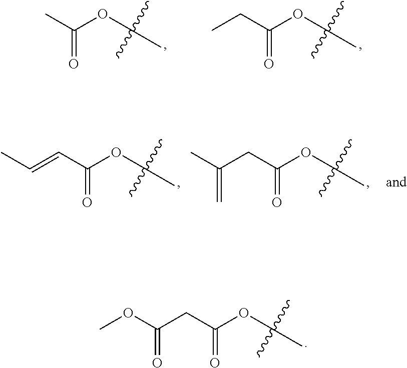

17. The compound of claim 16, or a pharmaceutically acceptable salt thereof, wherein the compound is selected from the group consisting of: 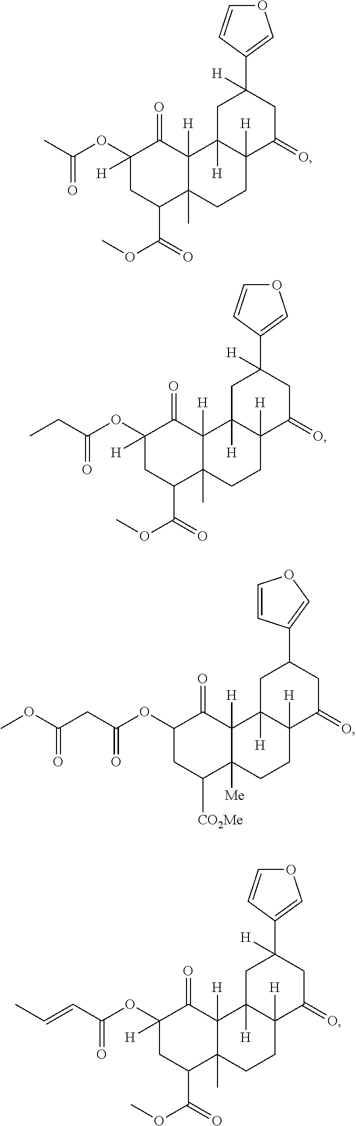 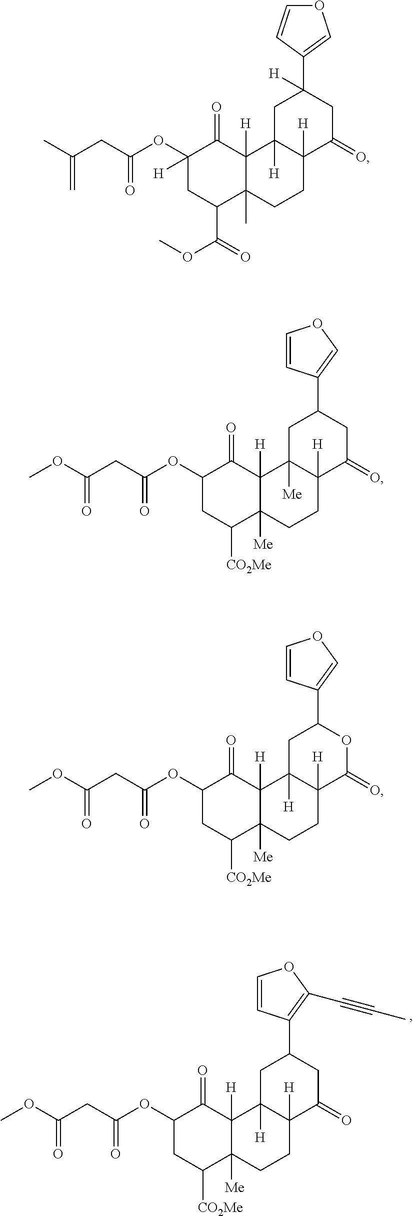 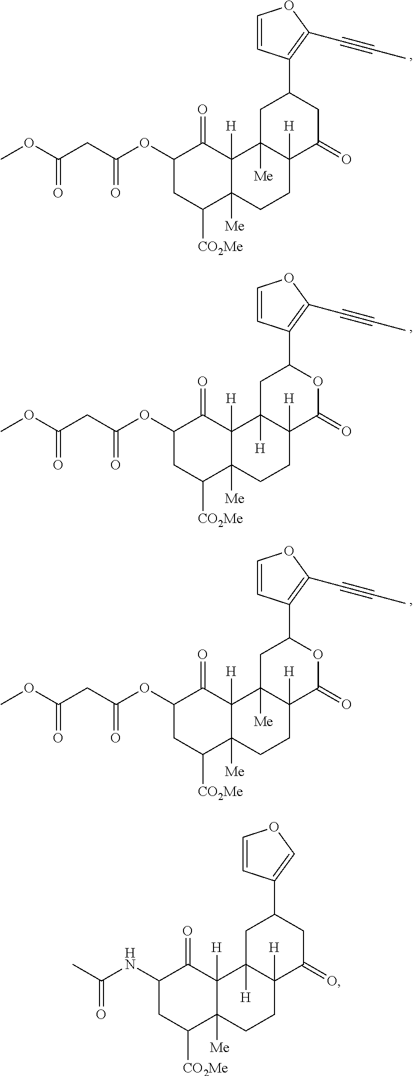 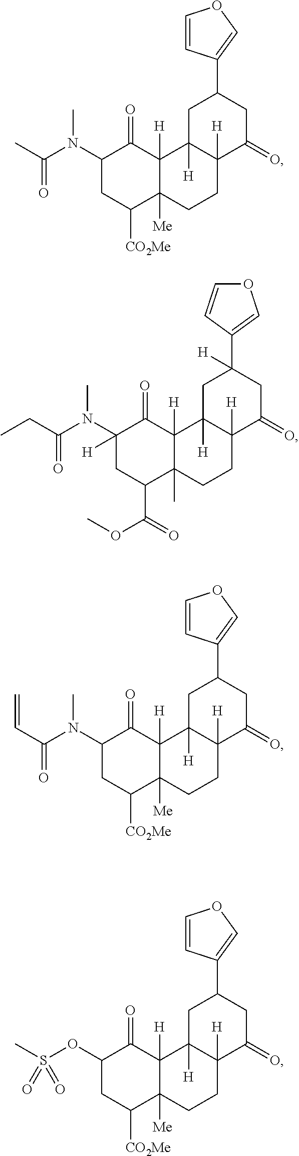 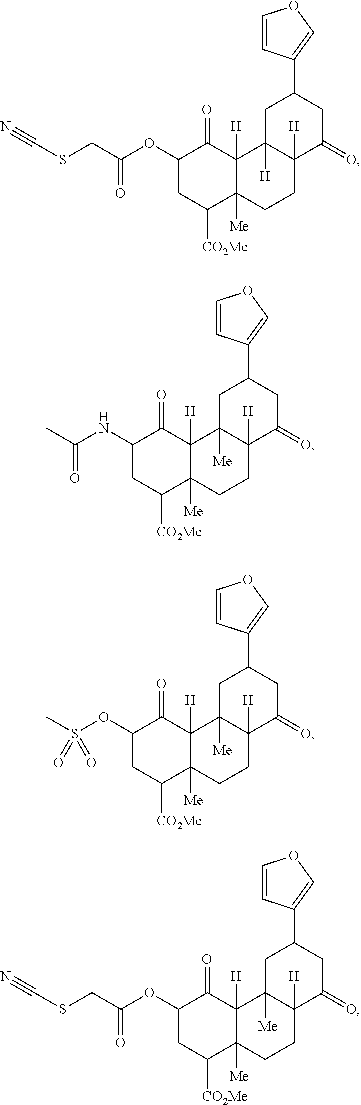 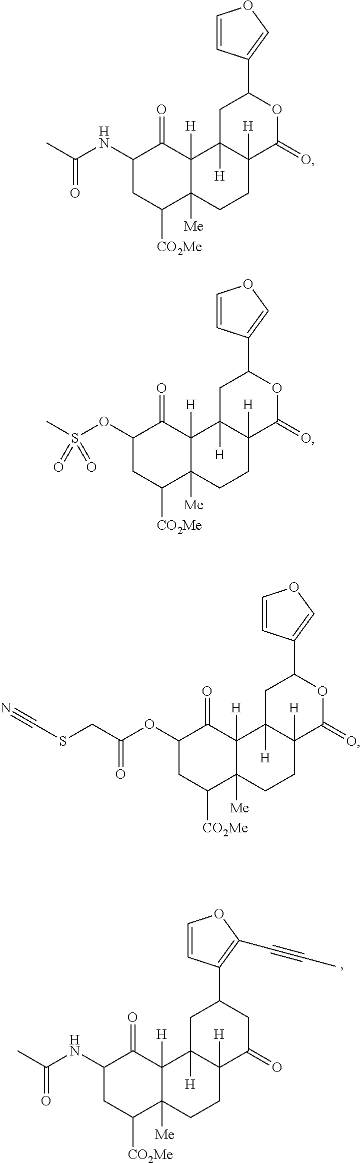 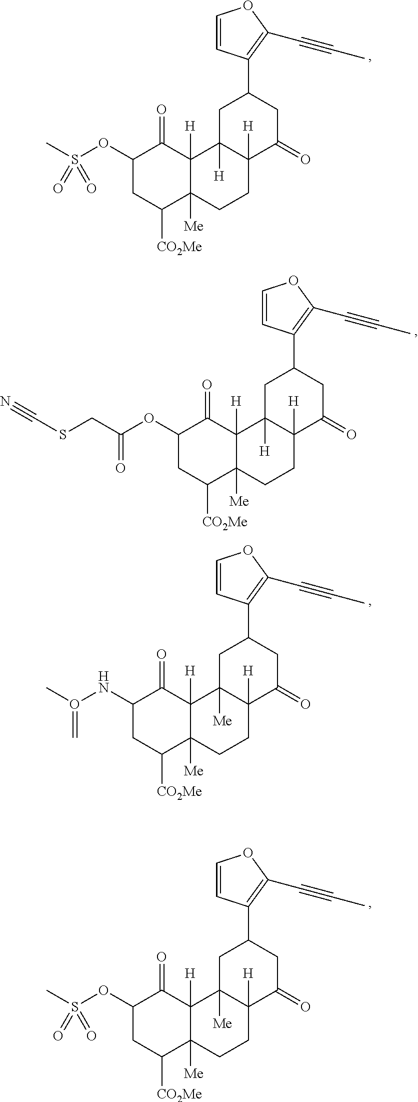 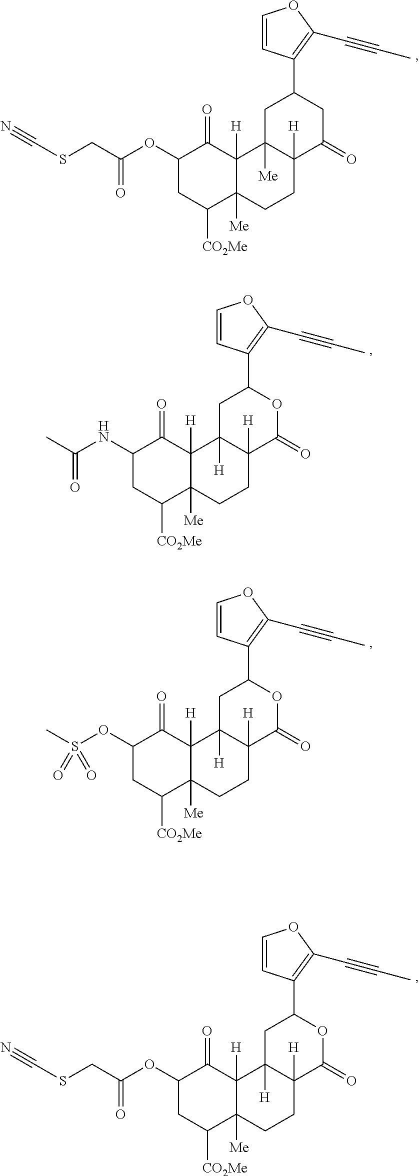 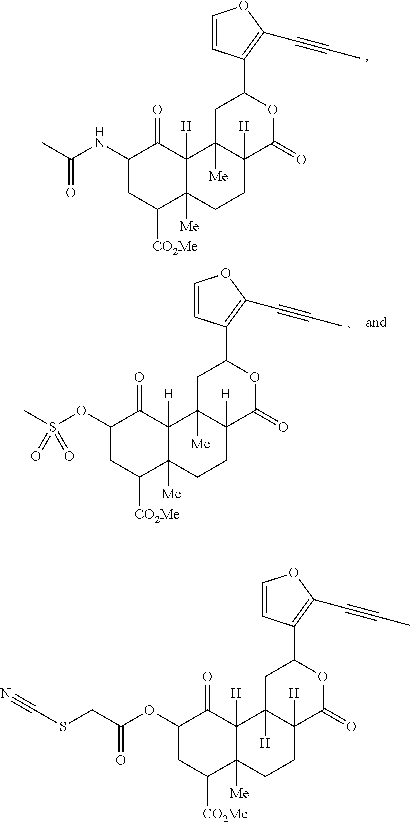
or a pharmaceutically acceptable salt of any of the foregoing.

18-20. (canceled)

21. The compound of claim 1, or a pharmaceutically acceptable salt thereof, having Formula II.a:

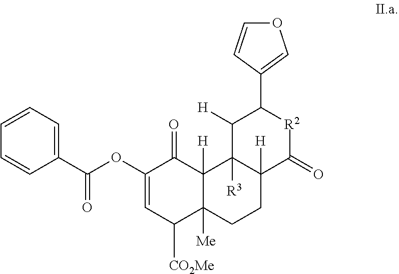

22. The compound of claim 21, or a pharmaceutically acceptable salt thereof, wherein R² is 0 or CH₂ and/or R³ is H or CH₃.

23. (canceled)

24. The compound of claim 22, or a pharmaceutically acceptable 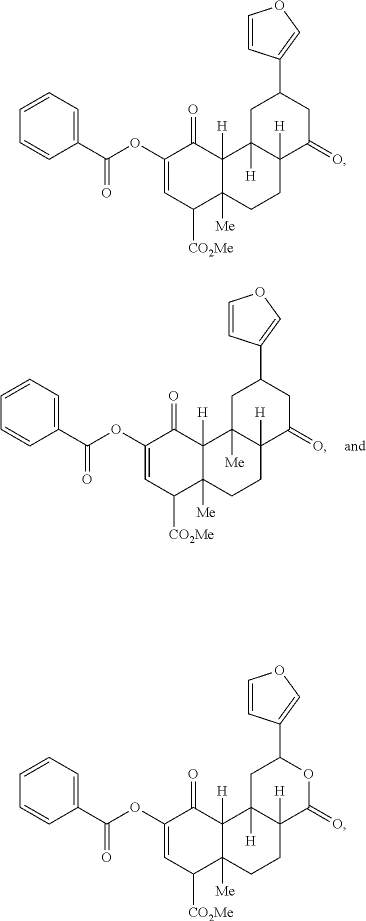
a pharmaceutically acceptable salt of any of the foregoing.

25. The compound of claim 1, or a pharmaceutically acceptable salt thereof, having Formula III.a:

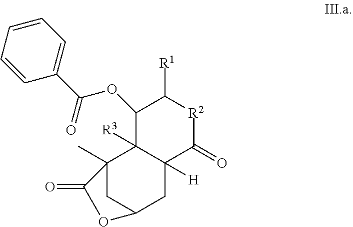

26. The compound of claim 25, or a pharmaceutically acceptable salt thereof, wherein R¹ is

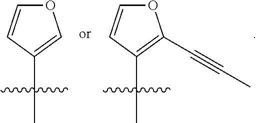

27. The compound of claim 25, or a pharmaceutically acceptable salt thereof, wherein R² is O or CH₂ and/or R³ is H or CH₃.

28. (canceled)

29. The compound of claim 27, or a pharmaceutically acceptable salt thereof, wherein the compound is selected from: 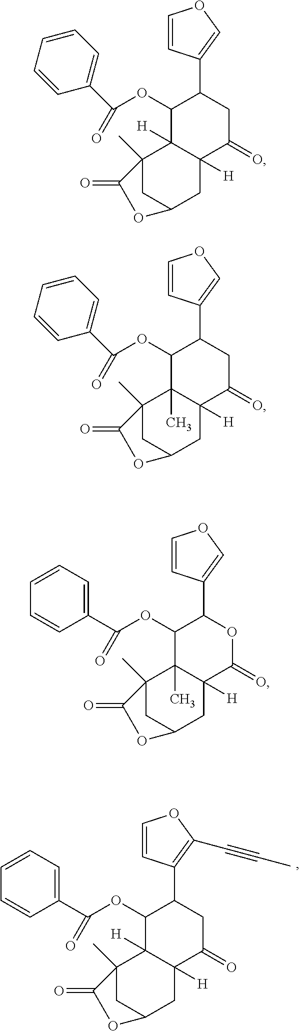 [SMILES: CC12CC(CC3C(=O)CC(c4ccoc4)C(OC(=O)c4ccccc4)C31)OC2=O ; CC12CC(CC3C(=O)CC(c4ccoc4)C(OC(=O)c4ccccc4)C31C)OC2=O ; CC12CC(CC3C(=O)OC(c4ccoc4)C(OC(=O)c4ccccc4)C31C)OC2=O ; CC#Cc1occc1C1CC(=O)C2CC3CC(C)(C(=O)O3)C2C1OC(=O)c1ccccc1] 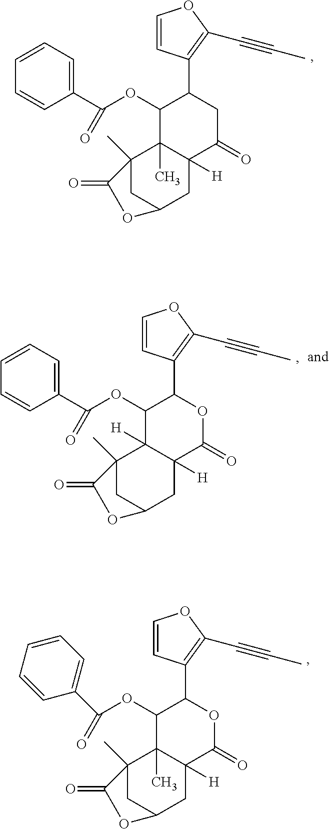 [SMILES: CC#Cc1occc1C1CC(=O)C2CC3CC(C)(C(=O)O3)C2(C)C1OC(=O)c1ccccc1 ; CC#Cc1occc1C1OC(=O)C2CC3CC(C)(C(=O)O3)C2C1OC(=O)c1ccccc1 ; CC#Cc1occc1C1OC(=O)C2CC3CC(C)(C(=O)O3)C2(C)C1OC(=O)c1ccccc1]
or a pharmaceutically acceptable salt of any of the foregoing.

30-36. (canceled)

37. A method for treating a disease or disorder that can be treated with a KOR agonist or MOR agonist, comprising administering an effective amount of the compound of claim 1, or a pharmaceutically acceptable salt thereof to a subject having the disease or disorder.

38-52. (canceled)
